SEQUENCE LISTING (30 sequences)

SEQ ID NO: 1 - DNA, 21 bp, Artificial Sequence
  Cbl-b siRNA SENSE
  misc_feature: synthetic
  misc_feature (20)..(21): t is deoxythymine
cccuuauuucaagcccugatt

SEQ ID NO: 2 - DNA, 19 bp, Artificial Sequence
  Cbl-b siRNA SENSE TARGET SEQ
  misc_feature: synthetic
cccttatttcaagccctga

SEQ ID NO: 3 - DNA, 21 bp, Artificial Sequence
  Cbl-b siRNA ANTISENSE
  misc_feature: synthetic
  misc_feature (20)..(21): t is deoxythymine
ucagggcuugaaauaagggtt

SEQ ID NO: 4 - DNA, 19 bp, Artificial Sequence
  Cbl-b siRNA ANTISENSE TARGET
  misc_feature: synthetic
tcagggcttgaaataaggg

SEQ ID NO: 5 - DNA, 21 bp, Artificial Sequence
  c-Cbl siRNA SENSE
  misc_feature: synthetic
  misc_feature (20)..(21): t is deoxythymine
cuguugacagacagacuaatt

SEQ ID NO: 6 - DNA, 19 bp, Artificial Sequence
  c-Cbl siRNA SENSE TARGET
  misc_feature: synthetic
ctgttgacagacagactaa

SEQ ID NO: 7 - DNA, 21 bp, Artificial Sequence
  c-Cbl siRNA ANTISENSE
  misc_feature: synthetic
  misc_feature (20)..(21): t is deoxythymine
uuagucugucugucaacagtt

SEQ ID NO: 8 - DNA, 19 bp, Artificial Sequence
  c-Cbl siRNA ANTISENSE TARGET
  misc_feature: synthetic
ttagtctgtctgtcaacag

SEQ ID NO: 9 - DNA, 21 bp, Artificial Sequence
  SHP-1 siRNA SENSE
  misc_feature: synthetic
  misc_feature (20)..(21): t is deoxythymine
cccugacccuguggaagcatt

SEQ ID NO: 10 - DNA, 19 bp, Artificial Sequence
  SHP-1 siRNA SENSE TARGET
  misc_feature: synthetic
ccctgaccctgtggaagca

SEQ ID NO: 11 - DNA, 21 bp, Artificial Sequence
  SHP-1 siRNA ANTISENSE
  misc_feature: synthetic
  misc_feature (20)..(21): t is deoxythymine
ugcuuccacagggucagggtt

SEQ ID NO: 12 - DNA, 19 bp, Artificial Sequence
  SHP-1 siRNA ANTISENSE TARGET
  misc_feature: synthetic
tgcttccacagggtcaggg

SEQ ID NO: 13 - DNA, 21 bp, Artificial Sequence
  N.S siRNA SENSE
  misc_feature: synthetic
  misc_feature (20)..(21): t is deoxythymine
uagcgacuaaacacaucaatt

SEQ ID NO: 14 - DNA, 21 bp, Artificial Sequence
  N.S siRNA ANTISENSE
  misc_feature: synthetic
  misc_feature (20)..(21): t is deoxythymine
uugauguguuuagucgcuatt

SEQ ID NO: 15 - DNA, 2949 bp, Homo sapiens
  misc_feature: Cbl-b, Accession number NM_170662.5
atggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatag

SEQ ID NO: 16 - protein, 982 aa, Homo sapiens [sequence omitted]

SEQ ID NO: 17 - protein, 906 aa, Homo sapiens
  MISC_FEATURE: c-Cbl, Accession number NP_005179.2
MAGNVKKSSGAGGGSGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 18 - DNA, 2721 bp, Homo sapiens
  misc_feature: c-Cbl, Accession number NM_005188.3
atggccggcaacgtgaagaagagctctggggccgggggcggcagcggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 19 - protein, 474 aa, Homo sapiens
  MISC_FEATURE: CBL-3, Accession number NP_036248.3
MALAVAPWGRQWEEARALGRAVRMLQRLEEQCVDPRLSVSPPSLRDLLPRTAQLLREVAHSRRAAGGGGPGGPGGSGDFLLIYLANLEAKSRQVAALLPPRGRRSANDELFRAGSRLRRQLAKLAIIFSHMHAELHALFPGGKYCGHMYQLTKAPAHTFWRESCGARCVLPWAEFESLLGTCHPVEPGCTALALRTTIDLTCSGHVSIFEFDVFTRLFQPWPTLLKNWQLLAVNHPGYMAFLTYDEVQERLQACRDKPGSYIFRPSCTRLGQWAIGYVSSDGSILQTIPANKPLSQVLLEGQKDGFYLYPDGKTHNPDLTELGQAEPQQRIHVSEEQLQLYWAMDSTFELCKICAESNKDVKIEPCGHLLCSCCLAAWQHSDSQTCPFCRCEIKGWEAVSIYQFHGQATAEDSGNSSDQEGRELELGQVPLSAPPLPPRPDLPPRKPRNAQPKVRLLKGNSPPAALGPQDPAPA

SEQ ID NO: 20 - DNA, 1425 bp, Homo sapiens
  misc_feature: CBL-3, Accession number NM_012116.4
atggctctggcggtggccccgtgggggcgacagtgggaagaggcccgcgccctgggccgggcagtcaggatgctgcagcgcctagaagagcaatgcgtcgacccccggctgtccgtgagtcccccttcgctgcgggacctgctgccccgcacagcgcagctgcttcgagaggtggcccattctcggcgggcggccggcggaggcggccccgggggtcccggcggctctggggactttctactcatctacctggccaatctggaggccaagagcaggcaggtggccgcgctgctgcctccccggggccgaaggagtgccaacgacgagctcttccgggcgggctccagactcaggcgacagctggccaagctggccatcatcttcagccacatgcacgcagagctgcacgcactcttccccgggggaaagtactgtggacacatgtaccagctcaccaaggcccccgcccacaccttctggagggaaagttgcggagcccggtgtgtgctgccctgggctgagtttgagtccctcctgggcacctgccaccctgtggaaccaggctgcacagccctggccttgcgcaccaccattgacctcacctgcagcgggcacgtgtccatcttcgagttcgacgtcttcaccaggctctttcagccatggccaacactcctcaagaactggcagctcctggcagtcaaccacccaggctacatggccttcctcacctatgatgaggtccaagagcgtctgcaggcctgcagggacaagccaggcagttacatcttccggcccagctgtactcgcctggggcagtgggccatcggctatgtgagctcagatggcagcatcctgcagaccatccctgccaacaaacccctgtcccaggtgctcctggagggacagaaggacggcttctacctctacccagatggaaagacccacaacccagacctgactgagctcggccaggcagaaccccagcagcgcatccacgtgtcagaggagcagctgcagctctactgggccatggactccacatttgagctctgcaagatctgtgctgagagcaacaaggatgtgaagattgagccgtgcgggcacctgctctgcagctgctgcctggctgcctggcagcactcggacagccagacctgccccttctgccgctgcgagatcaagggctgggaggccgtgagtatctaccagttccacggtcaggctactgctgaggactcagggaacagcagtgaccaggaaggcagggagttggagctggggcaggtgcccctttcggctcctccattgcccccacggccagatctgccccccaggaagcccagaaatgcccagccgaaagtgagactcctaaaggggaactcccctccagctgcgctgggaccccaggaccctgccccggcctga

SEQ ID NO: 21 - DNA, 1788 bp, Homo sapiens
  misc_feature: PTPN6 cDNA, transcript variant 1, NM_002831.5
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 22 - protein, 595 aa, Homo sapiens
  MISC_FEATURE: tyrosine-protein phosphatase non-receptor type 6 isoform 1, NP_002822.2
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 23 - DNA, 1794 bp, Homo sapiens
  misc_feature: PTPN6 transcript variant 2, cDNA, NM_080548.4
atgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 24 - DNA, 1875 bp, Homo sapiens
  misc_feature: PTPN6 transcript variant 3, cDNA, NM_080549.3
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaagagcttggagtctagtgcagggaccgtggctgcgtcacctgtgagacggggtggccagaggggactgccagtgccgggtccccctgtgctgtctcctgacctgcaccaactgcctgtacttgcccccctgcacccggctgcagacacaaggaggatgtgtatgagaacctgcacactaagaacaagagggaggagaaagtga

SEQ ID NO: 25 - protein, 597 aa, Homo sapiens
  MISC_FEATURE: tyrosine-protein phosphatase non-receptor type 6 isoform 2, NP_536858.1
MLSRGWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 26 - protein, 624 aa, Homo sapiens
  MISC_FEATURE: tyrosine-protein phosphatase non-receptor type 6 isoform 3, NP_536859.1
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKSLESSAGTVAASPVRRGGQRGLPVPGPPVLSPDLHQLPVLAPLHPAADTRRMCMRTCTLRTRGRRK

SEQ ID NO: 27 - DNA, 1782 bp, Homo sapiens
  misc_feature: PTPN11, transcript variant 1 cDNA, NM_002834.4
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatga

SEQ ID NO: 28 - DNA, 1383 bp, Homo sapiens
  misc_feature: PTPN11 transcript variant 2, cDNA, NM_080601.2
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcaggtga

SEQ ID NO: 29 - protein, 593 aa, Homo sapiens [sequence omitted]

SEQ ID NO: 30 - protein, 460 aa, Homo sapiens
  MISC_FEATURE: tyrosine-protein phosphatase non-receptor type 11 isoform 2, NP_542168.1
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR

(19) United States
(12) Patent Application Publication
(10) Pub. No.: US 2022/0096551 A1
(43) Pub. Date: Mar. 31, 2022

(54) COMBINATIONS, NANOPARTICLES AND METHODS FOR CONTROLLING NATURAL KILLER CELL ACTIVATION AND FUNCTION

(71) Applicant: BAR ILAN UNIVERSITY, Ramat Gan (IL)

(72) Inventor: Mira BARDA-SAAD, Ganney Tikva (IL)

(21) Appl. No.: 17/426,567

(22) PCT Filed: Jan. 28, 2020
(86) PCT No.: PCT/IL2020/050105

§ 371 Date: Jul. 28, 2021

Related U.S. Application Data
(60) Provisional application No. 62/797,373, filed on Jan. 28, 2019.

(51) Int. Cl. A61K 35/17; A61K 31/7105; A61K 9/14; A61P 35/00

(52) U.S. Cl. CPC A61K 35/17; A61P 35/00; A61K 9/14; A61K 31/7105

(57) ABSTRACT

The invention relates to combinations comprising at least one compound that inhibits the expression, activity and/or stability of at least one SHP protein and at least one Cbl protein, as well as nano-particles, cells and composition thereof. The combinations of the invention are used in methods for activating NK cells and/or T cells and for treating immune-related disorders.

### FIELD OF THE INVENTION

The present invention pertains to the field of molecular immunology, and specifically, personalized immunotherapy. More specifically, the present invention provides specific combinations, nanoparticles comprising said combinations and methods for activating NK cells, thereby conferring selective control on killing efficiencies of NK cell populations.

### BACKGROUND ART

References considered to be relevant as background to the presently disclosed subject matter are listed below:
[1] Watzl, C. & Long, E. O. Signal transduction during activation and inhibition of natural killer cells. Curr. Protoc. Immunol. Chapter 11, Unit 11.9B (2010).
[2] Long, E. O. Negative signaling by inhibitory receptors: the NK cell paradigm. Immunol. Rev. 224, 70-84 (2008).
[3] Stebbins, C. C. et al. Vav1 Dephosphorylation by the Tyrosine Phosphatase SHP-1 as a Mechanism for Inhibition of Cellular Cytotoxicity. Mol. Cell. Biol. 23, 6291-6299 (2003).
[4] Matalon, O. et al. Dephosphorylation of the adaptor LAT and phospholipase C-γ by SHP-1 inhibits natural killer cell cytotoxicity. Sci. Signal. 9, ra54 (2016).
[5] Campbell, K. S. Suppressing the killer instinct. Sci. Signal. 9, fs8 (2016).
[6] Matalon, O. et al. Actin retrograde flow controls natural killer cell response by regulating the conformation state of SHP-1. EMBO J. 37, e96264 (2018).
[7] WO 2018/134817.
[8] Cifaldi, L., Locatelli, F., Marasco, E., Moretta, L. & Pistoia, V. Boosting Natural Killer Cell-Based Immunotherapy with Anticancer Drugs: a Perspective. Trends Mol. Med. 23, 1156-1175 (2017).
[9] Granzin, M. et al. Shaping of Natural Killer Cell Antitumor Activity by Ex Vivo Cultivation. Front. Immunol. 8, 458 (2017).
[10] Guillerey, C., Huntington, N. D. & Smyth, M. J. Targeting natural killer cells in cancer immunotherapy. Nat. Immunol. 17, 1025-1036 (2016).
[11] Ruggeri, L. et al. Effectiveness of Donor Natural Killer Cell Alloreactivity in Mismatched Hematopoietic Transplants. Science (80). 295, 2097 LP-2100 (2002).
[12] Iliopoulou, E. G. et al. A phase I trial of adoptive transfer of allogeneic natural killer cells in patients with advanced non-small cell lung cancer. Cancer Immunol. Immunother. 59, 1781-1789 (2010).
[13] Becker, P. S. A. et al. Selection and expansion of natural killer cells for NK cell-based immunotherapy. Cancer Immunol. Immunother. 65, 477-484 (2016).
[14] Klingemann, H. Challenges of cancer therapy with natural killer cells. Cytotherapy 17, 245-249 (2017).
[15] Paolino, M. et al. The E3 ligase Cbl-b and TAM receptors regulate cancer metastasis via natural killer cells. Nature 507, 508-512 (2014).
[16] Messaoudene, M. et al. Patient's natural killer cells in the era of targeted therapies: Role for tumor killers. Front. Immunol. 8, 1-8 (2017).

Acknowledgement of the above references herein is not to be inferred as meaning that these are in any way relevant to the patentability of the presently disclosed subject matter.

### BACKGROUND OF THE INVENTION

Natural Killer (NK) cells are lymphocytes which constitute a major arm of the innate immune system, and are responsible for targeting and killing cancerous or pathogen i.e. virus, bacteria or parasite infected cells. NK cells directly kill target cells, and secrete cytokines and chemokines which aid in the recruitment of the adaptive immune response. More specifically, NK cells have been shown to regulate immune responses of other immune cells such as dendritic cells, T-cells, and macrophages. NK cells stochastically express a variegated array of germline encoded receptors which can either induce NK cell activation, or inhibit NK cell function. The response of NK cells towards potential targets depends on the integration of the cumulative signals deriving from stimulatory or inhibitory receptors. Ligation of activating receptors results in a protein tyrosine kinase (PTK) dependent pathway that is critical for NK cell activation [1]. Inhibitory receptor ligation, on the other hand, abrogates activating pathways via the recruitment of protein tyrosine phosphatases (PTPs), such as SHP-1/2 [2]. Recruitment of SHP-1 by inhibitory receptors, such as the killer-cell immunoglobulin-like (KIR) or CD94-NKG2A receptors following their ligation with human leukocyte antigen (HLA) results in dephosphorylation of key molecules, thus annulling NK cell activation. It was shown that upon NK cell inhibition, SHP-1 dephosphorylates the Rho family guanine nucleotide exchange factor (GEF) VAV1 [3]. The present inventors have shown that the transmembrane adapter protein, Linker for Activation of T-cells (LAT), and the regulators of intracellular calcium flux, phospholipase-Cγ1 (PLCγ1) and PLCγ2 [4]. The inventors have previously further showed that SHP-1 dephosphorylates LAT and PLC-γ during NK cell inhibition and as a result, reduces NK cell degranulation and the killing of its target cells [4]. Still further, the inventors recent study [4] showed that during NK cell inhibition by engagement of KIRs receptors with their ligands, LAT is ubiquitylated by the E3 ubiquitin ligases Cbl-b and c-Cbl [5]. This leads to their proteasomal degradation and thus abolishes the NK cell activation signaling pathway and its cytotoxicity capability.

In addition, the present inventors have recently demonstrated that during NK cell inhibition, SHP-1 binds to β-actin which affects SHP-1 active conformation state and enzymatic activity [6]. WO 2018/134817 [7], by the present inventors, provides specific compounds targeted at modulation of actin and/or myosin network dynamics in NK cells, thereby conferring selective control on killing efficiencies of NK cell populations. It is thus clear that NK cell inhibition is more complex and misunderstood that previously thought, and several additional inhibitory mechanisms regulate NK cell activity.

NK cells display innate and adaptive features that highlight their versatility and importance as an integral part of the immune system. Following the progress in understanding NK cells functions and mechanisms, they are becoming strong competitors to T cells in the immunotherapy field [8]. The ability of NK cells to rapidly mediate effector functions, without a need of prior antigen exposure, is a key distinguishing feature between mature NK cells and Cytotoxic T lymphocytes (CTLs), both efficient at mediating cytotoxicity. Only few NK cell-based immunotherapy approaches, such as adoptive transfer of autologous, have advantages over those using T cells [9]. Another therapeutic advantage of alloreactivity NK cells is their ability, by eliminating host antigen-presenting cells, to prevent GVHD [10, 11]. For example, the human NK cell line NK-92 is highly cytotoxic against a broad spectrum of malignant cells, and infusions of NK-92 cells are safe and well tolerated in cancer patients [12]. However, many current NK cells-based therapeutic approaches are challenged by difficulties in manufacturing large numbers of NK cells, low persistence, and the diminished activity of these cells in patients with cancer [13, 14]. Thus, passive approaches for improving NK cells function and killing are needed which minimizes the side effects. Furthermore, alteration of inhibitory receptors signaling and immunological check-points that limit NK cell function may be beneficial such as Src homology region 2 domain-containing Phosphatase-1 (SHP-1) and Casitas B-lineage lymphoma proto-oncogenes (Cbls) pathways [15].

As indicated above, NK cells have been proven to be potential agents for cell-based cancer therapies by their ability to mediate anti-tumor responses, without prior sensitization or recognition of specific tumor antigens [16]. However, the tumor microenvironment can suppress NK cell function resulting in tumor escape and disease progression. More specifically, tumors have developed several mechanisms to evade NK cell escape. These include, for example, downregulation of death receptors, induction of hypoxia, shedding of NK cell ligands, and induction of immune cell apoptosis. Chronic viral infections also induce downregulation of NK cell activity. Usually these viral “hijack” mechanisms involve expression of proteins that serve as ligands for inhibitory NK cell receptors or expression of soluble ligands that block activating receptors.

Thus, there is an unmet need in the art to control NK cells activation and inhibition threshold to overcome tumor immunosuppression and restore or even enhance NK cell activities. These needs are addressed by the present invention that provides modulators of NK cell activation adapted for personalized treatment.

### SUMMARY OF THE INVENTION

According to a first aspect, the invention relates to a combination comprising at least two compounds that specifically inhibit at least one of, the expression, activity and stability of at least one protein tyrosine phosphatase (PTP), specifically, at least one SHP protein and of at least one E3 ubiquitin-protein ligase, specifically, at least one member of the Casitas B-lineage Lymphoma (Cbl) E3 ubiquitin-protein ligase family and any nano- or micro-particle, micellar formulation, vehicle, matrix, or composition comprising said combination.

In yet another aspect, the invention relates to at least one nano- or micro-particle, micellar formulation, vehicle or matrix comprising at least one combination comprising at least two compounds that specifically inhibit at least one of, the expression, activity and stability of at least one PTP, and of at least one E3 ubiquitin-protein ligase. In some specific embodiments, the at least one nano- or micro-particle, micellar formulation, vehicle or matrix of the invention may comprise at least one combination comprising at least two compounds that specifically inhibit at least one of, the expression, activity and stability of at least one SHP protein and of at least one member of the Cbl family.

Another aspect of the invention relates to a pharmaceutical composition comprising as an active ingredient at least one combination according to the invention or any nano- or micro-particle, micellar formulation, vehicle, matrix or cell comprising the at least one combination as described by the invention.

In yet a further aspect, the invention relates to a method for activating inhibitory immunological synapse of hematopoietic cells, specifically, at least one of NK cells, T cells and B cells. In some particular and non-limiting embodiments, the method of the invention may be used for activating NK cells in an inhibitory immunological synapse (NKIS). In more specific embodiments, the method may comprise the step of contacting the NK cell with an activating effective amount of at least one combination comprising at least two compounds that specifically inhibit at least one of, the expression, activity and stability of at least one PTP, specifically, SHP protein and of at least one E3 ubiquitin-protein ligase, specifically, at least one member of the Cbl family, or any nano- or micro-particle, micellar formulation, vehicle matrix or composition comprising the combination of the invention, or any nano particles thereof.

In yet a further aspect, the invention relates to a method for treating, preventing, inhibiting, reducing, eliminating, protecting or delaying the onset of an immune-related disorder in a subject in need thereof. More specifically, the method of the invention may comprise the step of administering to the treated subject a therapeutically effective amount of at least one combination comprising at least two compounds that specifically inhibit at least one of, the expression, activity and stability of at least one PTP, specifically, SHP protein and of at least one E3 ubiquitin-protein ligase, specifically, at least one member of the Cbl family, or any nano- or micro-particle, micellar formulation, vehicle, matrix, cell or composition comprising the combination of the invention.

Still further aspect of the invention relates to at least one combination or any nano- or micro-particle, micellar formulation, vehicle, matrix, cell or composition comprising the combination, for use in a method for treating, preventing, inhibiting, reducing, eliminating, protecting or delaying the onset of an immune-related disorder in a subject in need thereof.

In yet a further aspect, the invention relates to a kit comprising:

As a first component (a), at least one compounds that specifically inhibit at least one of, the expression, activity and stability of at least one SHP protein; In a second component (b), at least one compounds that specifically inhibit at least one of, the expression, activity and stability of at least one member of the Cbl family; or any nano- or micro-particle, micellar formulation, vehicle or matrix, cell or composition comprising at least one of (a) and (b); and optionally (c), at least one additional therapeutic agent.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1C: Cbl-b, c-Cbl and SHP-1 siRNA gene silencing in YTS KIR2DL1

FIG. 1A: YTS KIR2DL1 cells were either mock transfected or transfected with 250 or 500 pmol of Cbl-b siRNA using Amaxa electroporation.

FIG. 1B: YTS KIR2DL1 cells were either mock transfected or transfected with 250 or 500 pmol of c-Cbl siRNA using Amaxa electroporation.

FIG. 1C: YTS KIR2DL1 cells were either mock transfected or transfected with 250 or 500 pmol of SHP-1 siRNA using Amaxa electroporation.

Following 48 hours, cells were lysed, and the nitrocellulose membranes were blotted with anti-Cbl-b, anti-c-Cbl or anti-SHP-1 antibodies. GAPDH served as a loading control. Densitometric analysis of the bands presented was performed using ImageJ and normalized to the GAPDH densitometry values. Relative expression of the three proteins compared to the mock control group is presented within the graph. The data represent three independent experiments. P values were calculated versus mock-treated control cells by two-tailed Student's t-test and are indicated by asterisks. *P≤0.05, **P≤0.005.

FIG. 2: SHP-1 and Cbls siRNA were used in combination in YTS KIR2DL1. YTS KIR2DL1 cells were mock-transfected or transfected with N.S siRNA or 250 pmol/each of Cbl-b siRNA, c-Cbl siRNA and SHP-1 siRNA, total of 750 pmol, using Amaxa electroporation. Following 48 hours, cells were lysed, and the membrane was blotted with anti-Cbl-b, anti-c-Cbl or anti-SHP-1 antibodies. GAPDH served as a loading control. Densitometric analysis of the bands presented was performed using ImageJ and normalized to the GAPDH densitometry values. Relative expression of the three proteins compared to N.S siRNA control group is presented within the graph. The data represent three independent experiments. P values were calculated versus N.S siRNA-treated control cells by two-tailed Student's t-test, and are indicated by asterisks. *P≤0.01, **P≤0.005.

FIG. 3: Combination of SHP-1 and Cbls siRNA increases intracellular calcium flux of NK cells following inhibitory interactions

YTS KIR2DL1 cells were mock-transfected (black curve), or transfected with either N.S siRNA (dark gray curve) or with SHP-1 and Cbls siRNA (light gray curve). Following 48 hours, cells were loaded with calcium-sensitive Fluo-3-AM and analyzed for basal intracellular calcium levels for 1 min. The NK cells were then mixed with 721.221 Cw4 target cells at 37° C. and the calcium levels were further analyzed by spectrofluorometry. A representative experiment out of three independent experiments is shown.

FIG. 4: SHP-1 and Cbls siRNA enhance NK cell degranulation

YTS KIR2DL1 cells were incubated with either 721.221 Cw4 or Cw7 target cells for 2 hours and analyzed by flow cytometry to determine the expression of CD107a positive cells. Expression of CD107a was measured by mean fluorescence Intensity (MFI). Relative expression of CD107a MFI was normalized to the mock-transfected sample following Cw4 incubation. The data represent three independent experiments. P values were calculated by two-tailed Student's t-test, and are indicated by asterisks. *P≤0.05, **P≤0.005. n.s—not significant.

FIG. 5: SHP-1 and Cbls siRNA enhance granzyme B release by NK cells

YTS KIR2DL1 cells were mock-transfected or transfected with N.S siRNA or SHP-1 and Cbls siRNA. Following 48 hours, cells were incubated with either 721.221 Cw4 or Cw7 target cells, the supernatant was then collected and granzyme B levels were evaluated using ELISA sandwich assay. The levels of granzyme B were quantified using standard recombinant granzyme B concentrations. Data are means±SEM of at least three independent experiments. P values were calculated by two-tailed Student's t-test, and are indicated by asterisks. *P≤0.01. n.s—not significant.

FIG. 6A-6B: NK cell-mediated killing is enhanced following gene silencing of SHP-1 and Cbls

FIG. 6A: YTS KIR2DL1 cells were transfected with N.S siRNA or SHP-1 and Cbls siRNA. Following 48 hours, cells were incubated with [³⁵S] Met-loaded 721.221 Cw4 target cells. After 5 hours of co-culture, the supernatant was collected, and the radioactive signal was measured. Data are means±SEM of at least three independent experiments.

FIG. 6B: YTS KIR2DL1 cells were mock-transfected or transfected with N.S siRNA or SHP-1 and Cbls siRNA and incubated with [³⁵S] Met-loaded 721.221 Cw4 or Cw7 target cells. After 5 hours of co-culture, the supernatant was collected, and the radioactive signal was measured, as described herein above for FIG. 6A. Data are means±SEM of at least three independent experiments. P values were calculated by two-tailed Student's t-test and are indicated by asterisks. *P≤0.05, **P≤0.005. n.s—not significant.

FIG. 7A-7B: synergistic effect on NK cell function BY gene silencing of SHP-1 and Cbls FIG. 7A. YTS KIR2DL1 cells were transfected with either N.S siRNA or with SHP-1 and Cbls siRNAs. Following 48 hours, cells were loaded with calcium-sensitive Fluo-3-AM and analyzed for basal intracellular calcium levels for 1 min. The NK cells were then mixed with 721.221 Cw4 target cells at 37° C. and the calcium levels were further analyzed by spectrofluorometry.

FIG. 7B. YTS KIR2DL1 cells were gene silenced to either SHP-1, Cbl-b c-Cbl individually or together. NK cells treated with N.S siRNA served as control. Following 48 hours, YTS-KIR2DL1 incubated with either 721.221 Cw4 or Cw7 target cells for 2 hours and analyzed by flow cytometry to determine the expression of CD107a positive cells. Expression of CD107a was measured by mean fluorescence Intensity (MFI). Relative expression of CD107a MFI was normalized to the mock-transfected sample following Cw4 incubation. Data are means±SEM of at least of three independent experiments. P values were calculated by two-tailed Student's t-test and are indicated by asterisks. *P≤0.05, **P≤0.005. n.s—not-significant.

FIG. 8A-8D: Specific staining of NKp46 in different cell lines using a monoclonal antibody

FIG. 8A: NK92-NKp46⁻ cells were stained with anti-NKp46 monoclonal antibody followed by staining with Alexa568-Fluor goat anti-mouse IgG secondary antibody.

FIG. 8B: NK92-NKp4⁺ cells were stained with anti-NKp46 monoclonal antibody followed by staining with Alexa568-Fluor goat anti-mouse IgG secondary antibody.

FIG. 8C: YTS KIR2DL1 cells were stained with anti-NKp46 monoclonal antibody followed by staining with Alexa568-Fluor goat anti-mouse IgG secondary antibody.

FIG. 8D: K562 cells were stained with anti-NKp46 monoclonal antibody followed by staining with Alexa568-Fluor goat anti-mouse IgG secondary antibody.

Cells were then analyzed using flow cytometry. A representative experiment out of three independent experiments is shown.

FIG. 9A-9C: Structure and characterization of target-specific liposomal NPs FIG. 9A: Schematic presentation of liposomal NPs' layers and composition.

FIG. 9B: Table presenting data of the NPs' characterization by diameter and zeta potential (Z-potential) of each layer together with a graph presenting NPs distribution of size.

FIG. 9C: Fluorescent image of the fluorescently-labeled NPs using confocal microscope.

FIG. 10: SHP-1 and Cbls siRNA-loaded NPs efficiently induce gene silencing in YTS KIR2DL1

YTS KIR2DL1 cells were incubated with NPs loaded with 500 pmol/each of Cbl-b, c-Cbl and SHP-1 siRNA or empty NPs (Neg. ctrl). Following 48 hours of incubation, cells were lysed, and the membrane was blotted with anti-Cbl-b, anti-c-Cbl or anti-SHP-1 antibodies. GAPDH served as loading control. Densitometric analysis of the bands presented was performed using ImageJ and normalized to the GAPDH densitometry values. Relative expression of the three proteins compared to negative control group is presented within the graph. The data represent three independent experiments. P values were calculated versus negative control-treated control cells by two-tailed Student's t-test, and are indicated by asterisks. *P≤0.05, **P≤0.0003.

FIG. 11A-11D: NKp46 antibody-coated NPs specifically target NK cells which express NKp46

FIG. 11A: NK92-NKp46⁻ cells were incubated with rhodamine-labeled NPs and analyzed using flow cytometry.

FIG. 11B: NK92-NKp46+ cells were incubated with rhodamine-labeled NPs and analyzed using flow cytometry.

FIG. 11C: YTS KIR2DL1 cells were incubated with rhodamine-labeled NPs and analyzed using flow cytometry.

FIG. 11D: K562 cells were incubated with rhodamine-labeled NPs and analyzed using flow cytometry.

FIG. 12A-12D: Confirming the internalization of the coated NPs in a NKp46-specific-manner

FIG. 12A: NK92-NKp46⁻ cells were incubated with rhodamine-labeled NPs and analyzed using confocal microscopy.:Upper panels wide-field:images. Lower panels single-cell images.

FIG. 12B: NK92-NKp46⁺ cells were incubated with rhodamine-labeled NPs and analyzed using confocal microscopy.:Upper panels wide-field:images. Lower panels single-cell images.

FIG. 12C: YTS KIR2DL1 cells were incubated with rhodamine-labeled NPs and analyzed using confocal microscopy.Upper panels wide-field:images. Lower panels single-cell images.

FIG. 12D K562 cells were incubated with rhodamine-labeled NPs and analyzed using confocal microscopy.Upper panels wide-field images. Lower panels single-cell images.

FIG. 13: SHP-1 and Cbls gene silencing using NPs loaded with siRNA increases intracellular calcium flux in NK cells following inhibitory interactions

YTS KIR2DL1 cells were served as negative control (gray curve) or treated with SHP-1 and Cbls siRNA-loaded NPs (dark gray curve). Following 48 hours, cells were loaded with calcium-sensitive Fluo-3-AM and analyzed for basal intracellular calcium levels for 1 min. The NK cells were then mixed with 721.221 Cw4 target cells at 37° C. and the calcium levels were further analyzed by spectrofluorometry.

FIG. 14: NPs loaded with SHP-1 and Cbls siRNA enhance NK cell degranulation

YTS KIR2DL1 cells were served as negative control or pretreated SHP-1 and Cbls siRNA-loaded NPs. Following 48 hours, cells were incubated with either 721.221 Cw4 or Cw7 target cells for 2 hours and analyzed by flow cytometry to determine the expression of CD107a positive cells. Expression of CD107a was measured by mean fluorescence Intensity (MFI). Relative expression of CD107a MFI was normalized to the negative control sample following Cw4 incubation. The data represent four independent experiments. P values were calculated by two-tailed Student's t-test, and are indicated by asterisks. *P≤0.05, **P≤0.01. n.s—not significant.

FIG. 15: SHP-1 and Cbls siRNA loaded NPs enhance granzyme B release by NK cells

YTS KIR2DL1 cells were served as negative control or treated with SHP-1 and Cbls siRNA-loaded NPs). Following 48 hours, cells were incubated with either 721.221 Cw4 or Cw7 target cells, the supernatant was then collected and granzyme B levels were evaluated using ELISA sandwich assay. The levels of granzyme B were quantified using standard recombinant granzyme B concentrations. Data are means±SEM of at least three independent experiments. P values were calculated by two-tailed Student's t-test, and are indicated by asterisks. *P≤0.05, **P≤0.01. n.s—not significant.

FIG. 16: NK cell-mediated killing is enhanced following gene silencing of SHP-1 and Cbls using NPs

YTS KIR2DL1 cells were serves as negative control or pretreated with SHP-1 and Cbls siRNA-loaded NPs. Following 48 hours, cells were incubated with [³⁵S] Met-loaded 721.221 Cw4 or Cw7 target cells. After co-culture for 5 hours, the supernatant was collected, and radioactive signal was measured. Data are means±SEM of at least of three independent experiments. P values were calculated by two-tailed Student's t-test, and are indicated by asterisks. *P≤0.05, **P≤0.005. n.s—not-significant.

FIG. 17: NKp46 antibody-coated NPs, either empty or loaded with siRNA, do not induce intracellular calcium flux in NK cells that are not subjected to inhibitory/activating interactions

YTS KIR2DL1 cells were incubated with empty NPs (light gray curve) or with SHP-1 and Cbls siRNA-loaded NPs (dark gray curve). Following 48 hours, cells were loaded with calcium-sensitive Fluo-3-AM and analyzed for intracellular calcium levels for 20 min at 37° C. YTS KIR2DL1 cells that were treated with PBS served as a negative control (gray curve). NK cells that were mixed with 721.221 Cw7 target cells served as a positive control (black curve). Calcium levels were analyzed by spectrofluorometry.

FIG. 18: Examining the effect of NKp46 antibody-coated NPs on the secretion of pro-inflammatory cytokines by healthy donor PBMCs

Freshly isolated PBMCs were treated with PBS as negative control, empty NPs, SHP-1 and Cbls siRNA-loaded NPs, or PHA and LPS as positive controls. Following incubation, supernatant from each sample was collected and the levels of human cytokines TNF-α, IL-6, IL-10 and IFN-γ were determined using ELISA. P values were calculated by two-tailed Student's t-test and are indicated by asterisks. *P≤0.008, **P≤0.0008, ***P≤0.0001.

FIG. 19: Schematic representation of the experimental timeline

Figure shows the timeline scheme of tumor engraftment and NPs injection.

FIG. 20A-20B: NPs containing SHP-1 and Cbls siRNA attenuate tumor growth in vivo FIG. 20A: Representative image of the mice at the end of the experiment.

FIG. 20B: Representative images of the tumors of the above mice at the end of the experiment.

FIG. 21A-21D: Therapeutic efficacy of SHP-1 and Cbls siRNA loaded-NPs following I.T administration

FIG. 21A: Tumor volume as was measured daily in NOD/SCID mice treated with 3×10⁶ irradiated YTS KIR2DL1 and 300 m NPs containing 500 pmol/each of Cbl-b, c-Cbl and SHP-1 siRNA (n=13), or group of mice treated with 3×10⁶ irradiated YTS KIR2DL1 and 300 μg NPs containing 1500 pmol of N.S siRNA that served as negative control (n=12).

FIG. 21B: Average growth rate of tumor from the first treatment.

P values were calculated by two-tailed Student's t-test, and are indicated by asterisks. *P≤0.04, **P≤0.001.

FIG. 21C: Following 24 hours from last treatment, extracted cells from tumors of either SHP-1 and Cbls or N.S siRNA loaded NKp46-NPs treated mice were analyzed by flow cytometry. Expression of CD107a was measured by mean fluorescence Intensity (MFI). Relative expression of CD107a MFI was normalized to the N.S siRNA loaded NPs treated sample.

FIG. 21D: Survival analysis in mice treated with NKs gene silenced for SHP-1 and Cbls using siRNA-loaded NPs or N.S siRNA-loaded NPs as demonstrated by Kaplan-Meier survival curve. Data are means±SEM of at least of three independent experiments. P values were calculated using the log-rank test and by two-tailed Student's t-test and are indicated by asterisks. *P≤0.05, n.s—not-significant.

### DETAILED DESCRIPTION OF THE INVENTION

Cancer immunotherapy attempts to harness the power and specific immune system characteristics for treating malignancies. Although cancer cells are less immunogenic than pathogens, the immune system is capable of recognizing and eliminating tumor cells. However, tumors frequently interfere with the development and function of immune responses. Thus, the challenge for immunotherapy is to develop strategies that effectively and safely augment current antitumor responses. NK cells, by cytotoxicity of target cells and production of cytokines that activate the adaptive immune system, represent a powerful immune defense against viral infections and tumor growth. Unlike other lymphocytes, NK cell activity is modulated by a large variety of inhibitory and activating receptors, and the balance between these signals determines whether the NK cell will react with the target cell. Furthermore, since NK cells kill tumor cells directly, without prior antigen exposure and without relying on antigen specificity, they are particularly effective.

Following the extensive and cumulative research of NK cells, and their yet unharnessed potential of cancer cells termination, NK cells are leaders in the immunotherapy field. Current NK cell therapeutic strategies still face many clinical challenges and to date no clinical data have clearly demonstrated its significant benefits in patients with malignancies. NK cell therapeutic approaches suffer various disadvantages, including the development of GVHD, lack of sufficient NK cell numbers to attain a therapeutic impact, the need for ex vivo expansion protocols, and the reduction of the NK cell cytolytic phonotype due to the isolation and treatment procedures. Furthermore, approaches aimed to target specific receptors, either by blocking or stimulating their activity, result in partial efficiency due to NK cells expression of a wide variety of and inhibitory receptors sharing similar signaling cascades. Therefore, although being able of blocking specific receptors, NK cells can still be activated or inhibited by other receptors, thus compromising the efficiency of improving NK cell cytotoxicity. The present invention addresses the limitations of current NK cell therapeutic approaches, while harnessing the NK cells' killing potential. As shown by the following Examples, the present invention provides herein a novel NK cell therapeutic strategy comprising lipid-based NPs loaded with siRNA, which target the key inhibitory regulators SHP-1 and Cbls, thus improving NK cell functioning in tumor cells killing and in killing of bacterially infected or virally infected cells, cells infected with fungal pathogen and cells infected by any of the pathogens disclosed herein after. Due to the lipid-based NPs passively enter the patients' bodies, there is no need to extract and perform manipulations on NK cells. Furthermore, there is no need for using cytokines and other substances involved in expending NK cells ex vivo. The therapeutic strategy of the present invention clearly diminishes risks of the patient and maintained the immune cells' system potency. Moreover, the strategy disclosed by the present invention provides specific tailor made therapy allowing personalized medicine by targeting specific repertoire of antigens (e.g., activating and/or inhibitory receptors) in immune-related cells (e.g., NK cells) of a specific patient, thereby specifically activating the immune response in the treated patient.

Thus, according to a first aspect, the invention relates to a combination comprising at least two compounds that specifically inhibit at least one of, the expression, activity and stability of at least one protein tyrosine phosphatase (PTP), and of at least one E3 ubiquitin-protein ligase, and any nano- or micro-particle, micellar formulation, vehicle, matrix or composition comprising the combinations of the invention.

In some specific embodiments, the combination of the invention may comprise at least two compounds that specifically inhibit at least one of, the expression, activity and stability of at least one Src homology region 2 (SH2) domain-containing phosphatase (SHP) and of at least one member of the Casitas B-lineage Lymphoma (Cbl) E3 ubiquitin-protein ligase family, or any vehicle, matrix, nano- or micro-particle, micellar formulation or composition comprising said combination.

As indicated herein, the combination of the invention comprises at least one compound that targets at least one member of the SHP family “Src homology region 2 (SH2) domain-containing phosphatase” (also Src tyrosine kinase activating tyrosine phosphatases) or simply SH2 domain-containing phosphatases refer to the most studied classical non-receptor tyrosine phosphatases (also non-receptor protein tyrosine phosphatases, PTPNs), SHP-1 and SHP-2. Both these phosphatases are characterized in that they possess a domain structure that consists of two tandem SH2 domains in its N-terminus followed by a PTP domain. This particular structure of the N-terminal of SHP-1 and SHP-2 is unique among other proteins with SH2 domains and confers them switching or auto-inhibiting property.

Further, the SHP-1 and SHP-2 phosphatases, sharing close sequence and structural homology. The major sequence differences between these two proteins are apparent in the approximately 100 amino acid residues at the extreme C-terminus, beyond the phosphatase catalytic domain. Thus when referring herein to the SH2 domain containing tyrosine phosphatases is meant the entire family of these proteins, including the four isoforms of SHP-1 and one isoform of SHP-2, and further the hematopoietic and non-hematopoietic cell specific isoforms of SHP-1, the latter arising from an alternative initiation site and varying by three amino acids at the N-terminus. This family further includes the longer 70 kDa form of SHP-1 (SHP-1L) that differs by 66 amino acids at the C-terminus due to alternative splicing of SHP-1 transcripts and subsequent shift of the reading frame. Relative to SHP-1, SHP-1L lacks one of the tyrosine phosphorylation sites and has a Pro-rich motif, a putative SH3-domain binding motif. As mentioned above, SHP-1 and SHP-2 share a characteristic N-terminal SH2 domain with phosphotyrosine binding sites facing outwards, which confer them the ability of auto-regulating phosphatase activity. The C-terminal SH2 domain has little interaction with the N-terminal SH2 domain or the catalytic domain. More specifically, in the ‘closed’ inhibited form (also inactive or ‘I’ state) the N-terminal SH2 domain forms extensive contacts with the catalytic domain through charge-charge-interactions, namely a part of the SH2 domain, the NXGDY/F motif, is inserted into the catalytic cleft of the enzyme, thus blocking access of substrates to the active site. Upon binding of a phosphopeptide, such as beta actin for example, the N-terminal SH2 domain undergoes an allosteric switch from the inactive ‘I’ state to the active ‘A’ state. This conformational change in the N-terminal SH2 domain disrupts the interaction between the SH2 domain and the phosphatase domain, and allows access of substrates.

The terms ‘SHP-1’ and ‘SHP-2’ refer herein to these genes product(s). In specific embodiments that are applicable to humans, these genes are denoted as, for SHP-1 as Protein Tyrosine Phosphatase Non-Receptor Type 6 (PTPN6), Hematopoietic Cell Protein-Tyrosine Phosphatase (HCP), Protein-Tyrosine Phosphatase 1C (PTP-1C, HPTP1C), SH-PTP1, and EC 3.1.3.48; and for SHP-2 as Protein Tyrosine Phosphatase, Non-Receptor Type 11 (PTPN11), Protein-Tyrosine Phosphatase 1D (PTP-1D), Protein-Tyrosine Phosphatase 2C (PTP2C), SH-PTP2, SH-PTP3 and EC 3.1.3.48; and further as the SHP-1 gene is located at the human chromosome 12p13.31 and the SHP-2 gene located at the human chromosome 12q24.13. These terms are applied herein to all known transcription isoforms and post translational modifications, such phosphorylation and ubiquitinations at specific residues.

In some embodiments, the human PTPN6 (or SHP 1) cDNA may refer to any one of transcript variant 1 NM_002831.5, that comprises the nucleic acid sequence as denoted by SEQ ID NO: 21, PTPN6 cDNA, transcript variant 2 NM_080548.4, that comprises the nucleic acid sequence as denoted by SEQ ID NO: 23 and PTPN6 cDNA, transcript variant 3 NM_080549.3, that comprises the nucleic acid sequence as denoted by SEQ ID NO: 24. In yet some further specific embodiments, the protein P29350-PTN6_HUMAN (SHP-1) referred to herein, may include the following isoforms (UniProtKB/Swiss-Prot), as denoted by RefSeq NP_002822.2 as denoted by SEQ ID NO: 22, NP_536858.1 as denoted by SEQ ID NO: 25, NP_536859.1 as denoted by SEQ ID NO: 26, respectively. Still further, the human PTPN11 (or SHP 2) cDNA may refer to any one of transcript variant 1, cDNA NM_002834.4, that comprises the nucleic acid sequence as denoted by SEQ ID NO: 27 and PTPN11 transcript variant 2, cDNA NM_080601.2, that comprises the nucleic acid sequence as denoted by SEQ ID NO: 28; and the protein Q06124-PTN11_HUMAN (UniProtKB/Swiss-Prot), and RefSeq NP_002825.3 as denoted by SEQ ID NO: 29, NP_542168.1 as denoted by SEQ ID NO: 30, respectively, having 597 amino acids and molecular mass of approximately 68.5 kDa.

Still further, the combination of the invention comprises at least one compound that targets and specifically inhibits at least one member of the CBL family. The mammalian CBL proteins family consists of three homologues: Casitas B-lineage Lymphoma-b (CBL-b), c-Casitas B-lineage Lymphoma (c-CBL) and Casitas B-lineage Lymphoma-3 (CBL-3). This family shares highly conserved regions in their N-terminal which encompass Tyrosin-kinase-binding (TKB), linker, and RING finger domains enabling CBL proteins to function as E3 ubiquitin ligases. In contrast, C-terminal regions are less well conserved. However, C-terminal regions enable interactions with SH2 and SH3 domains. CBL-b and c-CBL are mostly described as negative regulators in T cells. Yet, in NK cells, how CBL-b and c-CBL proteins regulate signaling responses remains unexplained. The inventors previously showed that during NK cell inhibition by engagement of KIRs receptors with their ligands, LAT is ubiquitylated by the E3 ubiquitin ligases CBL-b and c-CBL [4]. This leads to their proteasomal degradation and thus abolishes the NK cell activation signaling pathway and its cytotoxicity capability. CBL-b and c-CBL are thus considered as a second line of regulation in blocking LAT and PLC-γ dependent activation in NK cells. It should be appreciated that wherein indicated at least one “Cbls” “CBL protein” or the like throughout the application, it is meant at least one of any of the CBL proteins discussed herein (e.g., CBL-b, CBL 3, and c-CBL).

In yet some further specific embodiments, the human protein CBL-b as denoted by RefSeq NP_733762.2 may comprise the amino acid sequence as denoted by SEQ ID NO: 16, having 982 amino acids and molecular mass of approximately 109.4 kDa. In some embodiments, the human CBL-b gene, as denoted by NM_170662.5 may comprise the nucleic acid sequence as denoted by SEQ ID NO: 15.

In yet some further specific embodiments, the human protein c-CBL as denoted by RefSeq NP_005179.2 may comprise the amino acid sequence as denoted by SEQ ID NO: 17, having 906 amino acids and molecular mass of approximately 99.6 kDa. In some embodiments, the human c-CBL gene, as denoted by NM_005188.3 may comprise the nucleic acid sequence as denoted by SEQ ID NO: 18.

In yet some further specific embodiments, the human protein CBL-3 as denoted by RefSeq NP_036248.3 may comprise the amino acid sequence as denoted by SEQ ID NO: 19, having 474 amino acids and molecular mass of approximately 52.5 kDa. In some embodiments, the human c-CBL gene, as denoted by NM_012116.4 may comprise the nucleic acid sequence as denoted by SEQ ID NO: 20.

As noted above, the combinations of the invention may be any compound that specifically inhibit at least one of, the expression, activity and stability of at least one Src homology region 2 (SH2) domain-containing phosphatase (SHP) and of at least one member of the Casitas B-lineage Lymphoma (Cbl) E3 ubiquitin-protein ligase family.

A “Compound” is used herein to refer to any substance, agent (e.g., molecule), supramolecular complex, material, or combination or mixture thereof. A compound may be any agent that can be represented by a chemical formula, chemical structure, or sequence. Example of compounds applicable for the present invention, include, e.g., nucleic acid molecules (e.g., RNAi agents, antisense oligonucleotide, gRNAs, aptamers), small molecules, amino acid based molecules, for example, polypeptides, peptides, antibodies specific for SHP, and Cbl, that inhibit or disturb the activity thereof, lipids, polysaccharides, etc. It should be understood that any compound described in connection to the present aspect is also applicable in all aspects of the invention. It should be further understood that the invention encompasses the use of any of the described compounds or any combinations or mixtures thereof. In general, compounds may be obtained using any suitable method known in the art. The ordinary skilled artisan will select an appropriate method based, e.g., on the nature of the compound. A compound may be at least partly purified. In some embodiments a compound may be provided as part of a composition, which may contain, e.g., a counter-ion, aqueous or non-aqueous diluent or carrier, buffer, preservative, or other ingredient, in addition to the compound, in various embodiments. In some embodiments a compound may be provided as a salt, ester, hydrate, or solvate. In some embodiments a compound is cell-permeable, e.g., within the range of typical compounds that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

In yet some further embodiments, the inhibitory compound may be an antibody specifically directed against at least one SHP or at least one member of the Cbl family, and affects, specifically, reduces the amount, stability and function of at least one SHP or at least one member of the Cbl family. It should be noted that specific definition of the term antibodies as defined herein after in connection with other embodiments of the invention, is also relevant for these embodiments as well.

Still further, in certain embodiments, candidate compounds that specifically inhibit at least one of, the expression, activity and stability of at least one SHP and of at least one member of the Cbl family, can be screened from large libraries of synthetic or natural compounds. A compound to be tested may be referred to as a test compound or a candidate compound. Any compound may be used as a test compound in various embodiments. In some embodiments a library of FDA approved compounds that can be used by humans may be used. Compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbio screen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In some specific embodiments, the compound used by the combinations of the invention, that specifically inhibit at least one of, the expression, activity and stability of at least one SHP and of at least one member of the Cbl family, may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the combination of the invention may comprise at least two compounds, and at least one of these compounds may comprise at least one nucleic acid molecule. In some embodiments, each nucleic acid molecule is specifically directed against, or is specific for at least one of: (i) at least one of SHP-1 and SHP-2; and (ii) at least one of Cbl-b, c-Cbl and Cbl-3. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. It should be appreciated that the invention may further refer to polyribonucleotide. The term “polyribonucleotide” refers to a polynucleotide comprising two or more modified or unmodified ribonucleotides and/or their analogs. The term “polyribonucleotide” is used interchangeably with the term “oligoribonucleotide”. In more specific embodiments a nucleic acid molecule according to the invention may be an iRNA molecule, more specifically, dsRNA molecule.

In yet some further embodiments, the nucleic acid molecule of the combinations of the invention may be a ribonucleic acid (RNA) molecule or any nucleic acid sequence encoding said RNA molecule. In more specific embodiments, such RNA molecule may be at least one of a double-stranded RNA (dsRNA), an antisense RNA, a single-stranded RNA (ssRNA), gRNAs and a Ribozyme.

In certain embodiments, the combinations of the invention may comprise at least two dsRNA molecules, specifically, each directed against, or specific for, at least one of: (i) at least one of SHP-1 and SHP-2; and (ii) at least one of Cbl-b, c-Cbl and Cbl-3. In yet more specific embodiments, such dsRNA molecules, may be at least one of small interfering RNA (siRNA), MicroRNA (miRNA), short hairpin RNA (shRNA) and PIWI interacting RNAs (piRNAs).

Thus, in some embodiments, the at least two compounds of the invention may be nucleic acid molecules that may comprise at least one of a small interfering RNA (siRNA), a short hairpin RNA (shRNA), microRNA (miRNA), antisense oligonucleotide (ASO), locked nucleic acid (LNA), as well as other nucleic acids derivatives.

In some embodiments, the at least two compounds of the invention may be dsRNA molecules participating in RNA interference. More specifically, the dsRNA encompassed by the invention may be selected from the group consisting of small interfering RNA (siRNA), MicroRNA (miRNA), short hairpin RNA (shRNA), PIWI interacting RNAs (piRNAs). RNA interference (RNAi) is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the formation of partly or perfectly double-stranded RNA (dsRNA) between the target RNA and the siRNA/shRNA derived ‘guide” RNA strand. The expression of the gene is either completely or partially inhibited. As known in the art RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof. After binding to RISC, the guide strand is directed to the target mRNA cleaved between bases 10 and 11 relative to the 5′ end of the siRNA guide strand by the cleavage enzyme Argonaute-2 (AGO2). Thus, the process of mRNA translation can be interrupted by siRNA.

Thus, in some specific embodiments, the combinations of the invention may comprise at least one siRNA molecule that specifically target at least one member of the SHP family, and at least one siRNA molecule that specifically target at least one member of the CBL family. In more particular embodiments, siRNAs comprise a duplex, or double-stranded region, of about 5-50 or more, 10-50 or more, 15-50 or more, 5-45, 10-45, 15-45, 5-40, 10-40, 15-40, 5-35, 10-35, 15-35, 5-30, 10-30 and 15-30 or more nucleotides long. In yet some more particular embodiments, the siRNAs of the invention comprise a nucleic acid sequence comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product (i.e. RNA) molecule as herein defined. The strand complementary to a target RNA molecule is the “antisense guide strand”, the strand homologous to the target RNA molecule is the “sense passenger strand” (which is also complementary to the siRNA antisense guide strand). siRNAs may also be contained within structured such as miRNA and shRNA which has additional sequences such as loops, linking sequences as well as stems and other folded structures.

More specifically, the strands of a double-stranded interfering RNA (e.g., siRNA) may be connected to form a hairpin or stem-loop structure (e.g., shRNA). Thus, as mentioned above the compounds of the present invention may also be short hairpin RNA (shRNA).

According to other embodiments the compounds according to the present disclosure may be a micro-RNA (miRNA). miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA. The pre-miRNA is a substrate for a form of dicer that removes the miRNA duplex from the precursor, after which, similarly to siRNAs, the duplex can be taken into the RISC complex. Unlike, siRNAs, miRNAs bind to transcript sequences with only partial complementarity and usually repress translation without affecting steady-state RNA levels. Both miRNAs and siRNAs are processed by Dicer and associate with components of the RNA-induced silencing complex (RISC).

More specific embodiments relate to the compounds of the invention that may be at least one shRNA molecule. The term “shRNA”, as used herein, refers to an RNA agent having a stem-loop structure, comprising a first and second region of complementary sequence. The degree of complementarity and orientation of the regions being sufficient such that base pairing occurs between the regions. The first and second regions being joined by a loop region, the loop resulting from a lack of base pairing between nucleotides (or nucleotide analogs) within the loop region. Some of the nucleotides in the loop can be involved in base-pair interactions with other nucleotides in the loop.

An “antisense RNA” is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary” it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. Still further, in some embodiments, the inhibitory compound/s of the combinations of the invention may comprise an antisense oligonucleotide, or any derivatives thereof. In more specific embodiments such oligonucleotide is an antisense oligonucleotide (ASO). As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides. As used herein, “modified oligonucleotide” means an oligonucleotide comprising at least one modified nucleoside and/or at least one modified internucleoside linkage. Still further, “Antisense oligonucleotide” (AON, or ASO/s as used herein interchangeably) means an oligomeric compound, at least a portion of which is at least partially complementary to a target nucleic acid to which it hybridizes, for example, a target sequence within the nucleic acid sequence encoding at least one of, at least one SHP protein, and at least one Cbl protein. Such hybridization results in at least one antisense activity. In certain embodiments, the present invention provides antisense oligonucleotides of any of a variety of ranges of lengths. In certain embodiments, the present invention provides oligomeric compounds including oligonucleotides of any of a variety of ranges of lengths. In certain embodiments, the invention provides oligomeric compounds or oligonucleotides consisting of X to Y linked nucleosides or nucleotides, where X represents the fewest number of nucleosides in the range and Y represents the largest number of nucleosides in the range. In certain such embodiments, X and Y are each independently selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 and more nucleosides or nucleotides; provided that X<Y. In some embodiments, the oligonucleotides provided and used by the invention may comprise DNA, RNA, any derivatives thereof or any combinations thereof. More specifically, the currently used antisense oligonucleotides are rarely regular RNA or DNA oligonucleotide, as alternative antisense oligonucleotide chemistries have been developed to improve affinity, boost stability in the circulation and in target cells, and enhance cell penetration and nuclear accumulation. The non-bridging oxygen in the phosphate backbone may be replaced with a sulfur atom, generating phosphorothioate (PS) AONs. This modification enhances cellular uptake and improves resistance to nucleases but reduces the affinity of the AON to the target RNA. Addition of a methyl or a methoxyethyl group to the 2′-O atom of the ribose sugar (2′OMe and 2′OMOE, respectively) renders the AON-target RNA hybrid RNase H-resistant and increases the affinity of the AON for the target RNA. Most AONs have both the 2′O and the phosphorothioate (PS) modification (2′OMe-PS and 2′OMOE-PS) since they have a good safety profile and their synthesis is relatively inexpensive. “2′-OMe” or “2′-OCH₃” or “2′-O-methyl” each means a nucleoside comprising a sugar comprising an —OCH₃ group at the 2′ position of the sugar ring. “MOE” or “2′-MOE” or “2′-OCH₂CH₂OCH₃” or “2′-O-methoxyethyl” each means a nucleoside comprising a sugar comprising a —OCH₂CH₂OCH₃ group at the 2′ position of the sugar ring. In a different available oligonucleotide chemistry, a methylene bridge connects the 2′-O and the 4′-C of the ribose, forcing the nucleotide in the “endo” conformation, in what has been dubbed “locked nucleic acid” (LNA). This modification leads to a very high affinity for the target nucleic acid.

In addition to the described negatively charged oligonucleotides (2′OMe-PS, 2′OMOE-PS, and LNA), two more oligonucleotide chemistries may be used in attempts to inhibit at least one of the activity, expression and stability of at least one of, at least one SHP protein and at least one Cbl protein, in accordance with some embodiments of the invention, specifically, peptide nucleic acids (PNAs) and phosphorodiamidate morpholino oligomers (PMOs).

Still further, in some embodiments the inhibitory compounds of the invention may comprise at least one ribozyme. Ribozymes (ribonucleic acid enzymes) are RNA molecules that are capable of catalyzing specific biochemical reactions, similar to the action of protein enzymes. As used herein, the term “ribozyme” refers to a catalytically active RNA molecule capable of site-specific cleavage of target mRNA. In certain embodiments, a ribozyme is a Varkud satellite ribozyme, a hairpin ribozyme, a hammerhead ribozyme, or a hepatitis delta ribozyme.

In yet some further embodiments, the inhibitory compounds used in the combinations of the invention, as well as in any of the aspects of the invention, specifically, compound/s that inhibit the expression, activity and/or stability of at least one of, at least one SHP protein and at least one Cbl protein, may be based on any gene editing system, specifically programmable system, that is specifically directed against nucleic acid sequences comprised within the nucleic acid sequence encoding at least one of, at least one SHP protein and at least one Cbl protein. According to such embodiments, the inhibitory compound of the combinations of the invention may comprise at least one nucleic acid sequence that targets a modifier protein, for example, a nuclease or any fusion proteins thereof, to a target sequence within the nucleic acid sequence encoding at least one of, at least one SHP protein and at least one Cbl protein. Targeting the nucleic acid modifier to a specific target sequence, by a targeting molecule (such as a specific guide RNA), leads to specific binding to the target sequence and targeted manipulation (e.g., cleavage or any other modification), that leads to reduction in the expression, stability and/or activity of at least one of at least one SHP protein and at least one Cbl protein. Still further, in some embodiments the inhibitory compound is at least one guide RNA that guides at least one programmable engineered nucleases (PEN) to the target nucleic acid sequence as specified herein. In some embodiments, the PEN comprises at least one clustered regulatory interspaced short palindromic repeat (CRISPR)/CRISPR associated (cas) protein. Thus, according to some embodiments, the inhibitory compound used by the invention comprises: first (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, the inhibitory compound may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein, or any nucleic acid sequence encoding said gRNA; or any kit, composition or vehicle comprising at least one of (a) and (b).

Thus, in some embodiments, the Cas protein and the specific gRNA may be provided to and/or contacted with the target cell (e.g., hematopoietic cell, such as NK or T cell), or administered to the treated subject, either as a protein and gRNA, or alternatively, as nucleic acid sequences encoding these two elements, either in two separate nucleic acid molecules (e.g., two separate constructs), or in one nucleic acid molecule (e.g., a construct encoding both).

The term “programmable engineered nucleases (PEN)” as used herein also known as “molecular DNA scissors”, refers to enzymes either synthetic or natural, and used to replace, eliminate or modify target sequences in a highly targeted way. PEN target and cut specific genomic sequences (recognition sequences) such as DNA sequences. The at least one PEN may be derived from natural occurring nucleases or may be an artificial enzyme, all involved in DNA repair of double strand DNA lesions and enabling direct genome editing. In some alternative or additional embodiments the inhibitory compound according with the present disclosure encompasses also any nucleic acid molecule comprising at least one nucleic acid sequence encoding the PEN or any kit, composition or vehicle comprising the at least one PEN, or any nucleic acid sequence encoding the PEN.

In yet some further specific embodiments, such nucleases may include RNA guided nucleases such as CRISPR-Cas. However, it should be understood that in some alternative embodiments, other nucleases such as ZFN, TALEN, Homing endonuclease, Meganuclease, Mega-TALEN may be used by the methods of the invention for targeting at least one target nucleic acid sequence comprised within the nucleic acid sequence that encodes at least one of, at least one SHP protein and at least one Cbl protein.

More specifically, in some embodiments, the at least one PEN may be at least one of a mega nuclease, a zinc finger nuclease (ZFN), a transcription activator-like effector-based nuclease (TALEN), or a clustered regularly interspaced short palindromic repeats (CRISPR/Cas) system. In some embodiments, the at least one PEN may be a mega nuclease. Mega nucleases are endodeoxyribonucleases characterized by a large recognition site (double-stranded DNA sequences of 12 to 40 base pairs); such that this site generally occurs only once in any given genome. Meganucleases are specific naturally occurring restriction enzymes and include among others, the LAGLIDADG family of homing endonucleases, mostly found in the mitochondria and chloroplasts of eukaryotic unicellular organisms.

In some embodiments, the at least one PEN may be a megaTAL. MegaTALs are fusion proteins that combine homing endonucleases, such as LAGLIDADG family, with the modular DNA binding domains of TALENs.

In some alternative embodiments, the at least one PEN may be a zinc finger nuclease (ZFN). ZFNs are artificial restriction enzymes generated by fusing a zinc finger DNA-binding domain to a DNA-cleavage domain. Zinc finger domains can be engineered to target specific desired DNA sequences, enabling ZFN to target the target sequences within the target transcripts specified by the invention, thereby inhibiting the expression, activity and/or stability of at least one of, at least one SHP protein and at least one Cbl protein.

In yet some other embodiments, the at least one PEN may be a transcription activator-like effector-based nuclease (TALEN). TALEN are restriction enzymes that can be engineered to cut specific sequences of DNA. TALEN are made by fusing a TAL effector DNA-binding domain to a DNA cleavage domain (a nuclease which cuts DNA strands).

In some specific embodiments, the targeting of the target nucleic acid sequence that is comprised within the nucleic acid sequence that encodes at least one of, at least one SHP protein and at least one Cbl protein, may be mediated by a PEN that may comprise at least one clustered regulatory interspaced short palindromic repeat (CRISPR)/CRISPR associated (cas) protein system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. Thus, in some embodiments, the Cas protein may be a member of at least one of CRISPR-associated system of Class 1 and Class 2. In some embodiments, the cas protein may be a member of at least one of CRISPR-associated system of any one of type II, type I, type III, type IV, type V and type VI from E. coli, Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly any of the CRISPR systems disclosed herein. The CRISPR-Cas system, targets DNA molecules based on short homologous DNA sequences, called spacers that exist between repeats. These spacers guide CRISPR-associated (Cas) proteins to matching sequences within the target DNA, called proto-spacers, which are subsequently cleaved. The spacers can be rationally designed to form guide RNAs (gRNAs) that target any target DNA sequence, for example, the target sequence within the nucleic acid sequence that encodes at least one of, at least one SHP protein and at least one Cbl protein. It should be noted that the inhibitory compounds of the invention may comprise in some embodiments at least one gRNA targeted against at least one nucleic acid target that is comprised within at least one nucleic acid sequence that encodes at least one of, at least one SHP protein and at least one Cbl protein. Alternatively, the inhibitory compound of the invention may comprise any nucleic acid sequence encoding such gRNA. In some specific embodiment, the RNA guided DNA binding protein nuclease used by the invention may be a CRISPR Class 2 system. In yet some further particular embodiments, such class 2 system may be a CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cast and Cast. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage. It should be appreciated that any type II CRISPR-Cas systems may be applicable in the present invention, specifically, any one of type II-A, typeII-B or typeII-C. In more particular embodiments, at least one cas protein of type II CRISPR system used by the invention may be the cas9 protein, or any fragments, mutants, fusion proteins, variants or derivatives thereof (e.g., Cas9/Cpfl/CTc(1/2/3), SpCas9, SaCas9, engineered Cas9, and any mutants or fusion proteins thereof, for example, dCas9-Fok1, and the like). The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand brakes (DSBs). Still further, CRISPR type II system as used herein requires the inclusion of two essential components: a “guide” RNA (gRNA), that is comprised within the inhibitory compound/s of the invention, and a non-specific CRISPR-associated endonuclease (Cas9). Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”. In some embodiments, the gRNA of the invention may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers, or gRNA may comprise between about 20-35 nucleotides.

In yet some further embodiments, where the inhibitory compounds of the invention comprise at least one nucleic acid sequence encoding the gRNA, such encoding sequence may be designed to target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target protospacers (target sequences recognized by the gRNA) within the at least one nucleic acid sequence that encodes at least one of, at least one SHP protein and at least one Cbl protein. In CRISPR systems based on PAM (protospacer adjacent motif) sequence recognition like CRISPR Type II, the PAM is absolutely necessary for target binding and the exact sequence is dependent upon the species of Cas9 used. In certain embodiments, Cas9 from S. pyogenes may be used in the methods, cells, compositions, and kits of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS. Still further, it should be appreciated that type V CRISPR/Cas, including Cas12a, Cpfl (type VI), C2C1 (type V-B), Cas13 (type VI), specifically, C2C2 and CasRx and CasX, as well as any variants or fusion proteins thereof, is also applicable in the methods of the invention. In more specific embodiments, the gRNA comprised within the inhibitory compounds of the invention, targets the specific target sequence as disclosed by the invention and guides the CRISPR/Cas-protein, specifically, Cas9 to cleave, or perform other modification in the target site. The end result of Cas9-mediated DNA cleavage is a double strand break (DSB) within the target DNA. The resulting DSB may be then repaired by one of two general repair pathways, the efficient but error-prone Non-Homologous End Joining (NHEJ) pathway and the less efficient but high-fidelity Homology Directed Repair (HDR) pathway. In some specific embodiments, the targeted nucleic acid sequences specified above are repaired through the NHEJ pathway, resulting in most cases in alteration of the target sequence (deletions/insertions/non-sense mutations etc.), thereby inhibiting the expression, activity and/or stability of at least one of, at least one SHP protein and at least one Cbl protein.

As indicated above, the gene editing system used as the inhibitory compounds of the invention may be provided as nucleic acid molecules, specifically in a delivery vector or vehicle. However, it should be appreciated that any of the gene editing systems used, may be also administered as a protein complex, or alternatively, as a ribonucleoprotein complex. More specifically, when gene editing system is used by the invention such system may be delivered either as nucleic acid sequences encoding the components of this system, e.g., constructs comprising nucleic acid sequences that encode the CRISPR/Cas protein, for example, Cas9 and the specific gRNAs. However, it should be appreciated that the invention further encompasses in some embodiments thereof the option of using Cas9/gRNA Ribonucleoprotein complexes (Cas9 RNPs), that comprise purified Cas9 and purified gRNAs delivered as functional complexes. In some particular embodiments, purified gRNAs can be generated by PCR amplification of annealed gRNA oligos or in vitro transcription of a linearized gRNA containing plasmid. Cas9 (or any variant of Cas9) can be purified from bacteria through the use of bacterial Cas9 expression plasmids. In yet some further embodiments, the Cas9 RNP delivery to target cells may be carried out in some specific and non-limiting embodiments, via lipid-mediated transfection or electroporation.

In yet some further embodiments, the combination of the invention comprises at least two of: at least one siRNA molecule specific for, or specifically directed against SHP-1, at least one siRNA molecule specific for, or specifically directed against Cbl-b, and at least one siRNA molecule specific for, or specifically directed against c-Cbl.

Still further, in some embodiments, the combination of the invention may comprise at least one siRNA molecule specific for, or specifically directed against SHP-1, at least one siRNA molecule specific for, or specifically directed against Cbl-b and at least one siRNA molecule specific for, or specifically directed against c-Cbl.

In yet some further embodiments of the combinations of the invention, the siRNA molecule specifically directed against SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specifically directed against Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specifically directed against c-Cbl may comprise the nucleic acid sequence as denoted by any one of SEQ ID NO: 5 and SEQ ID NO: 7, or of any variants, homologs or derivatives thereof. Variants of the polynucleotides of the invention may have at least 80% sequence similarity to the entire sequence, often at least 85% sequence similarity, 90% sequence similarity, or at least 95%, 96%, 97%, 98%, or 99% sequence similarity or identity at the nucleic acid level, with the nucleic acid sequence of interest, such as the various polynucleotides of the invention. The term “derivative” is used to define nucleic acid sequence variants, and covalent modifications of a polynucleotide made use of in the present invention, e.g. of a specified sequence. The functional derivatives of any of the polynucleotides utilized according to the present invention, e.g. of a specified sequence of any one of the polynucleotides of SEQ ID NO: 1, 3, 5, 7, 9, preferably have at least about 65%, more preferably at least about 75%, even more preferably at least about 85%, most preferably at least about 95% overall sequence homology with the nucleic acid sequence of the polynucleotide as structurally defined above, e.g. of a specified sequence, more specifically, the entire nucleic acid sequence of the polynucleotides as denoted by any one of SEQ ID NO: 1, 3, 5, 7, 9, specifically, any homolog that retains the inhibitory effect on stability, expression and/or activity of any one of the SHP and the Cbl, as specified herein. “Homology” with respect to a native polynucleotide and its functional derivative is defined herein as the percentage of nucleic acid bases in the sequence that are identical with the bases of a corresponding polynucleotide Methods and computer programs for the alignment are well known. It should be appreciated that by the terms “insertions” or “deletions”, as used herein it is meant any addition or deletion, respectively, of nucleic acid bases to the polynucleotides used by the invention, of between 1 to 50 nucleic acid bases, between 20 to 1 nucleic acid bases, and specifically, between 1 to 10 nucleic acid bases. More particularly, insertions or deletions may be of any one of 1, 2, 3, 4, 5, 6, 7, 8, 9, or 10 nucleic acids, of any of the sequences disclosed herein. Still further, any derivatives or variants that retain the inhibitory effect of any of the specified sequences.

The terms “identical”, “substantial identity”, “substantial homology” or percent “identity”, in the context of two or more nucleic acids or polynucleotide sequences, refer to two or more sequences or subsequences that are the same or have a specified percentage of nucleic acid bases or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, specifically, over the whole sequence.

As indicated above, the compounds used in the combinations of the invention specifically inhibit the expression, stability and/or activity of at least one SHP and/or at least one Cbl protein. “Expression”, as used herein generally refers to the process by which gene-encoded information is converted into the structures present and operating in the cell. Therefore, according to the invention “expression” of a gene, specifically, may refer to transcription into a polynucleotide, translation into a protein, or even posttranslational modification of the protein. Protein “stability”, as used herein, refers to the physical (thermodynamic) stability, and chemical stability of the protein and relates to the net balance of forces, which determine whether a protein will be in its native folded conformation or a denatured state. More specifically, the levels of proteins within cells are determined not only by rates of synthesis as discussed above, but also by rates of degradation and the half-lives of proteins within cells that vary widely, from minutes to several days. In eukaryotic cells, two major pathways mediate protein degradation, the ubiquitin-proteasome pathway and lysosomal proteolysis.

Still further, the compounds used in the combination of the invention may in some embodiments inhibit the activity of any protein tyrosine phosphatase (PTP), specifically, the phosphatase activity of SHP on any of its substrates. More specifically, a Protein tyrosine phosphatase activity as used herein is meant the removal of phosphate groups from phosphorylated tyrosine residues on proteins. Protein tyrosine (pTyr) phosphorylation is a common post-translational modification that can create novel recognition motifs for protein interactions and cellular localization, affect protein stability, and regulate enzyme activity. As a consequence, maintaining an appropriate level of protein tyrosine phosphorylation is essential for many cellular functions. Tyrosine-specific protein phosphatases, for example, SHP proteins, catalyse the removal of a phosphate group attached to a tyrosine residue, using a cysteinyl-phosphate enzyme intermediate. SHP-1 dephosphorylates the Rho family guanine nucleotide exchange factor (GEF) VAV1, the transmembrane adapter protein, Linker for Activation of T-cells (LAT), and the regulators of intracellular calcium flux, phospholipase-Cγ1 (PLCγ1) and PLCγ2. Thus, in some embodiments, the compounds that are used in the combinations of the invention to inhibit the activity of SHP PTPs, affect, and specifically reduce the dephosphorylation of at least one of the above SHP substrates.

Still further, the compounds used for the combinations of the invention may affect Cbl proteins activity. Protein ubiquitination is a posttranslational modification that involves the covalent tethering of a small 76 amino acid protein called ubiquitin to target proteins. Ubiquitination mediates many cellular functions, which include signal transduction and the removal of proteins by the ubiquitin proteasome system (UPS). The initiation of protein ubiquitination typically requires an ATP-dependent enzymatic cascade that is initiated with the priming of a ubiquitin onto a ubiquitin activating enzyme (E1) and the transfer to a ubiquitin conjugating enzyme (E2). Ubiquitin is then covalently attached to a lysine residue on the target protein by an E3 ubiquitin ligase (E3) and this process can be repeated to create a series of ubiquitin chains. Ubiquitin chains can take various forms in length and configuration. The fate of these chains leads to multiple cellular functions, one of which provides a signal for the protein to undergo degradation by the UPS. More specifically, the Cbl proteins are E3 ubiquitin-protein ligases that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. Thus, E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome. Thus, the compound used in the combinations of the invention inhibit the transfer of ubiquitin from the E2 to the protein substrate by the Cbl family member, and therefore affect the stability of Cbl substrates.

As indicated above, the compounds used in the combinations of the invention and any compositions, kits and methods thereof, inhibit and/or reduce the expression, level, stability and/or activity of at least one of SHP and of at least one Cbl family member. More specifically, the terms “inhibition”, “moderation”, “reduction” or “attenuation” as referred to herein, relate to the retardation, restraining or reduction of the expression, levels, stability and/or activity of at least one SHP protein and/or at least one member of the Cbl family by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. It should be appreciated that 10%, 50%, 120%, 500%, etc., are interchangeable with “fold change” values, i.e., 0.1, 0.5, 1.2, 5, etc., respectively. 10%, 50%, 120%, 500%, etc., are interchangeable with “fold change” values, i.e., 0.1, 0.5, 1.2, 5, etc., respectively. Therefore, the term inhibit or decrease refers to an inhibition of about 2, 3, 4, 5, 6, 7, 8, 9, 10, 20, 30, 40, 50, 60, 70, 80, 90, 100, 200, 300, 400, 500, 600, 700, 800, 900, 1000 folds or more.

In some specific embodiments, the combination of the invention may be comprised within, encapsulated or enveloped by or within, at least one of a nano- or micro-particle, a micellar formulation, any vehicle, matrix, or a composition. It should be noted that vehicle, matrix, nano- or micro-particle, a micellar formulation or composition applicable in this aspect are in some embodiments those described herein after in connection with other aspects of the invention.

In yet another aspect, the invention relates to at least one nano- or micro-particle or micellar formulation or any vehicle, or matrix, comprising at least one combination comprising at least two compounds that specifically inhibit at least one of, the expression, activity and stability of at least one PTP, and of at least one E3 ubiquitin-protein ligase. In some specific embodiments, the at least one vehicle, matrix, nano- or micro-particle or micellar formulation of the invention may comprise at least one combination comprising at least two compounds that specifically inhibit at least one of, the expression, activity and stability of at least one SHP protein and of at least one member of the Cbl family.

In yet some further embodiments, the at least one vehicle, matrix, nano- or micro-particle or micellar formulation of the invention may comprise any of the combinations described by the invention. It should be noted that in some embodiments, the compounds of the combination of the invention may be any inhibitory nucleic acid molecules, SMCs, aptamers, peptide, or any combinations thereof, that specifically inhibit at least one of: (i) at least one of SHP-1 and SHP-2; and (ii) at least one of Cbl-b, c-Cbl and Cbl-3. More specifically, in some embodiments, the combination comprised within the vehicle, matrix, nano- or micro-particle or micellar formulation of the invention may comprise at least two compounds that comprise at least one nucleic acid molecule. More specifically, each of the nucleic acid molecules is specific for or specifically directed against one of at least one of: (i) at least one of SHP-1 and SHP-2; and (ii) at least one of Cbl-b, c-Cbl and Cbl-3.

In yet some further embodiments, the nucleic acid molecule of the combination comprised within the nano- or micro-particle or micellar formulation or vehicle or matrix of the invention, may be RNA molecules or any nucleic acid sequence encoding the RNA molecules. In more specific embodiments, such RNA molecules may be at least one of a dsRNA, an antisense RNA, a ssRNA, a Ribozyme and a guide RNA.

In certain embodiments, the combinations comprised within the nano- or micro-particle or micellar formulation or vehicle or matrix of the invention may comprise at least two dsRNA molecules. More specifically, each of the dsRNA molecules may be directed against or specific for one of at least one of: (i) at least one of SHP-1 and SHP-2; and (ii) at least one of Cbl-b, c-Cbl and Cbl-3. In yet more specific embodiments, such dsRNA molecules, may be at least one of siRNA, miRNA, shRNA and piRNAs.

In yet some further embodiments, the combinations comprised within the nano- or micro-particle or micellar formulation or vehicle or matrix of the invention may comprise at least two of: at least one siRNA molecule specifically directed against, or specific for SHP-1, at least one siRNA molecule specifically directed against Cbl-b, and at least one siRNA molecule specific for, or specifically directed against c-Cbl.

Still further, in some embodiments, the combinations comprised within the nano- or micro-particle or micellar formulation or vehicle or matrix of the invention may comprise at least one siRNA molecule specifically directed against, or specific for SHP-1, at least one siRNA molecule specifically directed against, or specific for Cbl-b and at least one siRNA molecule specifically directed against or specific for c-Cbl.

In yet some further embodiments, of the combination comprised within the nano- or micro-particle or micellar formulation of the invention, the siRNA molecule specifically directed against, or specific for SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specifically directed against, or specific for Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specifically directed against, or specific for c-Cbl may comprise the nucleic acid sequence as denoted by any one of SEQ ID NO: 5 and SEQ ID NO: 7, or of any variants, homologs or derivatives thereof.

In yet some further embodiments of the nano- or micro-particle or micellar formulation or vehicle or matrix of the invention, the at least one combination may be encapsulated within the intra-nanoparticle core and/or cavity of the nanoparticle of the invention. More specifically, in some embodiments, the compounds of the combinations of the invention, specifically, the siRNAs of the invention may be surrounded, enveloped, encapsulated, entrapped and comprised within a nanoparticle, specifically, within the inner core and/or cavity of a nano- or microparticle. In more specific embodiments, at least one targeting moiety is connected and/or associated directly, or indirectly, with the outer nanoparticle surface of the nano-micro particle, vehicle, matrix, microparticle or micellar formulation of the invention.

In more specific embodiments, the at least one targeting moiety of the nano- or micro-particle or micellar formulation or vehicle or matrix of the invention, may be any affinity molecule, for example, at least one of an antibody, an aptamer, a ligand (e.g., for an activating receptor, or alternatively, a ligand for inhibitory receptor) or any combinations thereof, that specifically recognizes and binds at least one molecule expressed on the surface of at least one hematopoietic cell. As indicated above, in some embodiments, the targeting moiety used by the nano-particles of the invention may comprise an antibody or any antigen binding fragments thereof. In yet some further embodiments, the antibody used as a targeting moiety for the vehicle, matrix, nano- or micro-particle or micellar formulation of the invention, may be any one of: full length antibody, antibody fragment, single-chain variable fragment (scFv), bi-specific antibody, tri-specific antibody and variable new antigen receptor antibody (V-NAR).

The term “antibody” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH₁, CH₂ and CH₃. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics. Still further, as indicated above, an “antigen-binding domain” can comprise or consist of an antibody or antigen-binding fragment of an antibody.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

The antibody suitable for the targeting moiety of the nanoparticle of the invention may also be a bi-specific antibody (such as Bi-specific T-cell engagers-BiTEs) or a tri-specific antibody. The antibody suitable for the invention may also be a variable new antigen receptor antibody (V-NAR). VNARs are a class of small, immunoglobulin-like molecules from the shark immune system. Humanized versions of VNARs could be used to bind protein epitopes that are difficult to access using traditional antibodies.

As noted above, in some embodiments, the targeting moiety of the nanoparticle of the invention may comprise aptamers that specifically recognize and bind at least one member of the SHP family, and at least one member of the CBL family. As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers are usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides.

In some embodiments, the aptamer that may be applicable herein may optionally comprise a spacer between the nucleic acid sequence and the reactive group. The spacer may be an alkyl chain such as (CH₂)_{6/12}, namely comprising six to twelve carbon atoms.

In some embodiments, the at least one target moiety of the nano-particles of the invention, specifically recognizes and binds and thereby targets at least one molecule expressed on the surface of at least one hematopoietic cell.

“Hematopoietic cells” are cellular blood components all derived from hematopoietic stem cells in the bone marrow. It should be appreciated that in certain embodiments, hematopoietic cells as used herein include cells of the myeloid and the lymphoid lineages of blood cells. More specifically, myeloid cells include monocytes, (macrophages and dendritic cells (DCs)), granulocytes (neutrophils), basophils, eosinophils, erythrocytes, and megakaryocytes or platelets. The Lymphoid cells include T cells, B cells, and natural killer (NK) cells. Thus, in certain embodiments, the cells treated by the compounds of the invention may be any hematopoietic cell described herein. Generally, blood cells are divided into three lineages: red blood cells (erythroid cells) which are the oxygen carrying, white blood cells (leukocytes that are further subdivided into granulocytes, monocytes and lymphocytes) and platelets (thrombocytes).

In yet some embodiments, the at least one target moiety of the nano-particles of the invention, specifically recognizes and binds and thereby targets at least one molecule expressed on the surface of a lymphocyte.

“Lymphocytes” as used herein, are mononuclear nonphagocytic leukocytes found in the blood, lymph, and lymphoid tissues. They comprise the body's immunologically competent cells and their precursors. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells. In yet some further embodiments, the hematopoietic cell recognized or targeted by the targeting moiety of the nano- or micro-particle or micellar formulation or vehicle or matrix of the invention, may be a natural killer (NK) cell. Still further, in some embodiments, the nanoparticle, vehicle, matrix, microparticle or micellar formulation of the invention, may be associated directly or indirectly by the outer nanoparticle surface thereof with at least one antibody, at least one aptamer or any combinations thereof, that specifically recognize and bind at least one of at least one NK cell activating receptor and at least one NK cell inhibitory receptor or any combinations thereof. Natural killer (NK) cells are a type of cytotoxic lymphocytes that are critical to the innate immune system in providing rapid responses to viral-infected cells and tumor formation. In contrast to CTLs, NK cells do not express T cell antigen receptors (TCR) or pan T marker CD3 or surface immunoglobulins (Ig) B cell receptors, instead they express the surface markers CD16 (FcγRIII) and CD56 in humans (NK1.1 or NK1.2 in mice), up to 80% of human NK cells also express CD8. Further, NK cells are effectors of innate immunity in expressing activating and inhibitory NK receptors, which play an important function in self-tolerance and in sustaining NK activity.

Natural killer cell cytolysis of target cells and cytokine production is controlled by a balance of inhibitory and activating signals, which are facilitated by NK cell receptors. NK cell inhibitory receptors are part of either the immunoglobulin-like (IgSF) superfamily or the C-type lectin-like receptor (CTLR) superfamily. Members of the IgSF family comprise the human killer cell immunoglobulin-like receptor (KIR) and the Immunoglobulin-like transcripts (ILT).

Killer-cell immunoglobulin-like receptors (KIRs), are a family of type I transmembrane glycoproteins expressed on the plasma membrane of natural killer (NK) cells and a minority of T cells. They regulate the killing function of these cells by interacting with major histocompatibility (MHC) class I molecules, which are expressed on all nucleated cell types. KIR receptors can distinguish between major histocompatibility (MHC) class I allelic variants, which allows them to detect virally infected cells or transformed cells. Most KIRs are inhibitory, meaning that their recognition of MHC molecules suppresses the cytotoxic activity of their NK cell. Only a limited number of KIRs are activating, meaning that their recognition of MHC molecules activates the cytotoxic activity of their cell.

Inhibitory receptors recognize self-MHC class I molecules on target self-cells, causing the activation of signaling pathways that stop the cytolytic function of NK cells. Self-MHC class I molecules are always expressed under normal circumstance. According to the missing-self hypothesis, inhibitory MR receptors recognize the downregulation of MHC class I molecules in virally-infected or transformed self-cells, leading these receptors to stop sending the inhibition signal, which then leads to the lysis of these unhealthy cells. Because natural killer cells target virally infected host cells and tumor cells, inhibitory KIR receptors are important in facilitating self-tolerance.

KIR inhibitory receptors signal through their immunoreceptor tyrosine-based inhibitory motif (ITIM) in their cytoplasmic domain. When inhibitory KIR receptors bind to a ligand, their ITIMs are tyrosine phosphorylated and protein tyrosine phosphatases, including SHP-1, are recruited. In some embodiments, inhibitory NK cells receptors may include but are not limited to at least one of Killer-cell immunoglobulin-like receptor (KIR), Programmed cell death protein 1 (PD-1), cytotoxic T-lymphocyte-associated protein 4 (CTLA-4), Ly49, NKRP1A, CD94, KIRNKG2A, T cell immunoreceptor with Ig and ITIM domains (TIGIT), CD96, T-cell immunoglobulin and mucin-domain containing-3 (TIM-3), Lymphocyte-activation gene 3 (LAG-3), Carcinoembryonic antigen-related cell adhesion molecule 1 (CEACAM1), Leukocyte-associated immunoglobulin-like receptor 1 (LAIR-1), Leukocyte immunoglobulin-like receptor subfamily B member 1 (LILRB1), and any derivatives, splice variants, homologs and orthologs thereof or any combinations thereof.

Activating receptors recognize ligands that indicate host cell aberration, including induced-self antigens (which are markers of infected self-cells and comprise MICA, MICB, and ULBP, all of which are related to MCH class 1 molecules), altered-self antigens (MHC class I antigens laden with foreign peptide), and/or non-self (pathogen encoded molecules). The binding of activating KIR receptors to these molecules causes the activation of signaling pathways that cause NK cells to lyse virally infected or transformed cells.

Activating receptors do not have the immunoreceptor tyrosine-base inhibition motif (ITIM) characteristic of inhibitory receptors, and instead contain a positively charged lysine or arginine residue in their transmembrane domain (with the exception of KIR2B4) that helps to bind DAP12, an adaptor molecule containing a negatively charged residue as well as immunoreceptor tyrosine-based activation motifs (ITAM). Activating KIR receptors include KIR2DS, KIR2DL1, KIR3DS and CD244 (Cluster of Differentiation 244), also known as Natural Killer Cell Receptor 2B4. In some embodiments, activating NK cells receptors may include but are not limited to at least one of Natural cytotoxicity triggering receptor 1 (NCR1, NKp46), Natural cytotoxicity triggering receptor 2 (NCR2, NKp44), Natural cytotoxicity triggering receptor 3 (NCR3, NKp30), tumor necrosis factor receptor superfamily 7 (TNFRSF7, CD27), Lymphocyte function-associated antigen 1 (LFA-1), cluster of differentiation 16 (CD16), NKG2D, Cytotoxic And Regulatory T Cell Molecule (CRTAM), DNAX Accessory Molecule-1)(DNAM-1), NKp80 (NKp80, also known as killer cell lectin-like receptor subfamily F, member 1 (KLRF1)), and any derivatives, splice variants, homologs and orthologs thereof.

NK cells express receptors for MHC class I molecules comprising the C-type lectin-like receptors, CD94/NKG2. CD94/NKG2 receptors are expressed on majority of NK cells and a subset of CD8+ T cells. Five different molecular species of NKG2 (NKG2A, B, C, E and H) have been reported to form disulfide-linked heterodimers with invariant CD94. NKG2A and B, which are products from a single gene by alternative splicing, have two immunoreceptor tyrosine-based inhibitory motifs (ITIM) in their cytoplasmic domains and form inhibitory receptors complexed with CD94. NKG2C, E and H, the latter two of which are also products from a single gene, as well as NKG2C, have positively charged residues within their transmembrane regions. NKG2C and possibly NKG2E and H interact with the adapter molecule DAP12, and act as activating receptors, when heterodimerized with CD94.

NK cells also play a role in adaptive immune response in their ability to readily adjust to the immediate environment and formulate antigen-specific immunological memory, fundamental for responding to secondary infections with the same antigen. Thus NK cells are acting in both the innate and adaptive immunity, which makes them particularly useful targets for the combinations of the present invention. In more specific embodiments, the NK cell activating receptor may be at least one of Natural cytotoxicity triggering receptor 1 (NCR1, NKp46), Natural cytotoxicity triggering receptor 2 (NCR2, NKp44), Natural cytotoxicity triggering receptor 3 (NCR3, NKp30), tumor necrosis factor receptor superfamily 7 (TNFRSF7, CD27), Lymphocyte function-associated antigen 1 (LFA-1), cluster of differentiation 16 (CD16), NKG2D, Cytotoxic And Regulatory T Cell Molecule (CRTAM), DNAX Accessory Molecule-1) (DNAM-1), NKp80, 2B4 also known as CD244 (Cluster of Differentiation 244) and any derivatives, splice variants, homologs and orthologs thereof, and wherein said NK cell inhibitory receptor is at least one of Killer-cell immunoglobulin-like receptor (KIR), Programmed cell death protein 1 (PD-1), CTLA-4, Ly49, NKRP1A, CD94, NKG2A, T cell immunoreceptor with Ig and ITIM domains (TIGIT), CD96, T-cell immunoglobulin and mucin-domain containing-3 (TIM-3), Lymphocyte-activation gene 3 (LAG-3), Carcinoembryonic antigen-related cell adhesion molecule 1 (CEACAM1), Leukocyte-associated immunoglobulin-like receptor 1 (LAIR-1), Leukocyte immunoglobulin-like receptor subfamily B member 1 (LILRB1), and any derivatives, splice, homologs and orthologs variants thereof or any combinations thereof.

In yet some further specific embodiments, the NK cell activating receptor may be at least one of NKp46 (denoted by the human RefSeqs: NM_001145457, NM_001145458, NM_001242356, NM_001242357, NM_004829, also known as Natural cytotoxicity triggering receptor 1 (NCR1), CD335, LY94, NK-p46, NKP46,), NKp44 (denoted by the human RefSeqs: NM_004828, NM_001199509, NM_001199510, also known as Natural cytotoxicity triggering receptor 2 (NCR2), CD336, LY95, NK-p44), NKp30 (denoted by the human RefSeqs: NM_001145466, NM_001145467, NM_147130, also known as Natural cytotoxicity triggering receptor 3 (NCR3), 1C7, CD337, LY117, MALS), NKp80 (RefSeq. NM_016523.3, encoding the protein as denoted by NP_057607.1 the also known as KLRF1), CD27 (denoted by the human RefSeq: NM_001242, also known as 5152, 5152. LPFS2, T14, TNFRSF7, Tp55, CD27 molecule), LFA-1 (RefSeq. NM_002209.3, encoding the protein as denoted by NP_002200.2), CD16 (denoted by the human RefSeqs: NM_000569, NM_000570 also known as FCGR3, FCG3, Fc₇RIII), NKG2D (denoted by the human RefSeq: NM_007360, also known as KLRK1, CD314, D12S2489E, KLR, NKG2-D, NKG2D, killer cell lectin like receptor K1), CRTAM, DNAM-1 (denoted by the human RefSeqs: NM_001303618, NM_001303619, NM_006566, also known as CD226, DNAM-1, DNAM1, PTA1, TLiSA1, CD226 molecule), 2B4 (denoted by the human RefSeqs: NM_001166663, NM_001166664, NM_016382, also known as CD244, 2B4, NAIL, NKR2B4, Nmrk, SLAMF4, CD244 molecule), and any derivatives and splice variants thereof. In yet some further embodiments, the NK cell inhibitory receptor may be at least one of KIR (RefSeq: NM_014218.3, encoding the protein as denoted by NP 055033.2, also referred to herein as KIR2DL1, killer cell immunoglobulin-like receptors), PD-1 (denoted by the human RefSeq: NM_005018, also known as PDCD1, CD279, PD-1, PD1, SLEB2, hPD-1, hPD-1, hSLE1, Programmed cell death 1), Ly49 (denoted by the human RefSeqs: NM_008462.5 and NM_006611, also known as Klra2, Ly49b; Klra30, and as KLRA1; Ly49; LY49L; Ly-49L, KLRAP1, respectively), NKRP1A (denoted by the human RefSeq: NM_002258, also known as KLRB1, CD161, CLEC5B, NKR, NKR-P1, NKR-P1A, hNKR-P1A, killer cell lectin like receptor B1), CD94 (denoted by the human RefSeqs: NM_001114396, NM_002262, NM_007334, NM_001351060, NM_001351062, also known as KLRD1), NKG2A (also known as CD94), TIGIT (Gene ID: 201633), CD96 (denoted by the human RefSeqs: NM_005816, NM_198196, NM_001318889, also known as TACTILE, CD96 molecule), TIM-3 (Gene ID: 84868, also known as TIM3; CD366; KIM-3; SPTCL; TIMD3; Tim-3; TIMD-3; HAVcr-2), LAG-3 (denoted by the human RefSeq: NM_002286, also known as LAG3, CD223, lymphocyte activating 3), CEACAM1 (denoted by the human RefSeqs: NM_001024912, NM_001184813, NM_001184815, NM_001184816, NM_001205344 also known as CECAM, BGP, BGP1, BGPI, AND CEA cell adhesion molecule 1), LAIR-1 (denoted by the human RefSeq: NM_001289023, NM_001289025, NM_001289026, NM_001289027, NM_002287 also known as CD305, LAIR1), LILRB1 (denoted by the human RefSeq: NM_001081637, NM_001081638, NM_001081639, NM_001278398, NM_001278399, also known as ILT2, CD85J, ILT-2, ILT2, LIR-1, LIR1, MIR-7, MIR7, PIR-B, PIRB, leukocyte immunoglobulin like receptor B1), BTLA (denoted by the human RefSeq: NM_001085357, NM_181780, also known as BTLA, BTLA1, CD272, B and T lymphocyte), CTLA4 (denoted by the human RefSeq: NM_001037631, NM_005214, also known as ALPSS, CD, CD152, CELIAC3, CTLA-4, GRD4, GSE, IDDM12). It should be understood that the NK activating or inhibitory receptors as specified herein are also applicable for all other aspects of the invention.

In some embodiments, the at least one nano- or micro-particle, micellar formulation, vehicle or matrix of the invention are suitable for hematopoietic cell that is a T cell. According to these embodiments, the nano- or micro-particle, micellar formulation, vehicle or matrix is connected directly or indirectly by the outer nanoparticle surface thereof with at least one antibody, at least one aptamer or any combinations thereof, that specifically recognize and bind at least one of at least one T cell activating receptor and at least one T cell inhibitory receptor or any combinations thereof. In more specific embodiments, the T cell activating receptor is any one of cluster of differentiation 3 (CD3), Cluster of Differentiation 28 (CD28), Cluster of Differentiation 69 (CD69), cluster of differentiation 4 (CD4), cluster of differentiation 8 (CD8), cluster of differentiation 137 (CD137), and T cell inhibitory receptor is at least one of Programmed cell death protein 1 (PD-)1, B- and T-lymphocyte attenuator (BTLA), luster of differentiation 160 (CD160), Cluster of Differentiation 244 (CD244, 2B4), cytotoxic T-lymphocyte associated protein 4 (CTLA-4), Lymphocyte-activation gene 3 (LAG-3), T cell immunoreceptor with Ig and ITIM domains (Tigit), Carcinoembryonic antigen-related cell adhesion molecule 1 (biliary glycoprotein) (CECAM), T cell immunoglobulin and mucin domain-containing protein 3 (TIM3), Leukocyte-associated immunoglobulin-like receptor 1 (Lair1), Leukocyte immunoglobulin-like receptor subfamily B member 3 (LILRB3 or PirB), Platelet endothelial cell adhesion molecule (PECAM-1), cluster of differentiation-22 (CD22, Siglec 2), Sialic acid-binding Ig-like lectin 7 (Siglec 7), Sialic acid-binding Ig-like lectin 9 (Siglec 9), Killer cell lectin-like receptor subfamily G member 1 (KLRG1), Ig-Like Transcript 2 (ILT2), Killer-cell immunoglobulin-like receptor 2DL1 (KIR2DL/3DL), Cluster of Differentiation 72 (CD72), Cluster of Differentiation 94 (CD94, NKG2A) and cluster of differentiation 5 (CD5).

In some particular embodiments, the T cell activating and or inhibitory receptors may be the receptors a specified herein, specifically, at least one of CD3 (RefSeq NM_000732, NM_000733, NM_000073), CD28 (RefSeq NM_001243077, NM_001243078, NM_006139), CD69 (RefSeq NM_001781), CD4 (NM_000616, NM_001195014, NM_001195015, NM_001195016, NM_001195017), CD8 (NM_001768, NM_172099), CD137 (NM_001561, also known as TNFRSF9, 4-1BB, CD137, CDw137, ILA, tumor necrosis factor receptor superfamily member 9), and T cell inhibitory receptor is at least one of PD-1 (NM_005018, also known as PDCD1, CD279, PD-1, PD1, SLEB2, hPD-1, hPD-1, hSLE1, Programmed cell death 1), BTLA (NM_001085357, NM_181780, also known as BTLA, BTLA1, CD272, B and T lymphocyte), CD160 (NM_007053, also known as CD160, BY55, NK1, NK28, CD160 molecule), 2B4 (NM_001166663, NM_001166664 and NM_016382, also known as CD244 and 2B4, as well as NAIL, NKR2B4, Nmrk, SLAMF4), CTLA-4 (NM_001037631, NM_005214, also known as ALPSS, CD, CD152, CELIAC3, CTLA4, GRD4, GSE, IDDM12), LAG-3 (NM_002286, also known as LAG3, CD223, lymphocyte activating 3), Tigit (Gene ID: 201633), CECAM (NM_001024912, NM_001184813, NM_001184815, NM_001184816, NM_001205344 also known as CEACAM1, BGP, BGP1, BGPI, AND CEA cell adhesion molecule 1), Tim3 (Gene ID: 84868, also known as TIM3; CD366; KIM-3; SPTCL; TIMD3; Tim-3; TIMD-3; HAVcr-2), Lairl (NM_001289023, NM_001289025, NM_001289026, NM_001289027, NM_002287 also known as CD305, LAIR-1), LILRB3 (NM_001081450, NM_006864, NM_001320960, also known as PIRB, CD85A, HL9, ILT-5, ILT5, LILRA6, LIR-3, LIR3, PIR-B), PECAM1 (NM_000442, also known as PECAM1, CD31, CD31/EndoCAM, GPIIA′, PECA1, PECAM-1, endoCAM, platelet and endothelial cell adhesion molecule 1, PCAM-1), CD22 (NM_024916, NM_001185099, NM_001185100, NM_001185101, NM_001278417, also known as SIGLEC2, CD22 molecule Siglec 2), Siglec 7 (NM_001277201, NM_014385, NM_016543, also known as AIRM1, CD328, CDw328, D-siglec, QA79, SIGLEC-7, SIGLEC19P, SIGLECP2, p′75, p75/AIRM1), Siglec 9 (NM_001198558, NM_014441, CD329, CDw329, FOAP-9, OBBP-LIKE, siglec-9, sialic acid binding Ig like lectin 9), KLRG1 (NM_001329099, NM_001329101, NM_001329102, NM_001329103, NM_005810, also known as 2F1, CLEC15A, MAFA, MAFA-2F1, MAFA-L, MAFA-LIKE), ILT2 (NM_001081637, NM_001081638, NM_001081639, NM_001278398, NM_001278399, also known as LILRB1, CD85J, ILT-2, ILT2, LIR-1, LIR1, MIR-7, MIR7, PIR-B, PIRB, leukocyte immunoglobulin like receptor B1), KIR2DL/3DL (NM_014218, also known as CD158A, KIR-K64, KIR221, NKAT, NKAT-1, NKAT1, p58.1, killer cell immunoglobulin like receptor, two Ig domains and long cytoplasmic tail 1, KIR2DS1, KIR2DL3), CD72 (uniPort P21854-1), CD94 (NM_001114396, NM_002262, NM_007334, NM_001351060, NM_001351062, also known as KLRD1), NKG2A (also known as CD94) and CD5 (NM_014207, NM_001346456, also known as LEU1, T1, CD5 molecule). It should be understood that the T cell activating or inhibitory receptors as specified herein are also applicable for all other aspects of the invention.

In yet some further embodiments, the hematopoietic cell activated by the methods of the invention may be at least one T cell. More specifically, a “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells. More specifically, thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Thymocytes are classified into a number of distinct maturational stages based on the expression of cell surface markers. The earliest thymocyte stage is the double negative (DN) stage (negative for both CD4 and CD8), which more recently has been better described as Lineage-negative, and which can be divided into four sub-stages. The next major stage is the double positive (DP) stage (positive for both CD4 and CD8). The final stage in maturation is the single positive (SP) stage (positive for either CD4 or CD8).

In some embodiments, the invention relates to any of the activating and/or inhibitory receptors of NK cells or T cells, and to any derivatives, splice, homologs and orthologs variants thereof or any combinations thereof. Thus, in some embodiments, the activating or inhibitory receptors of the NK cells targeted by the targeting moiety of the nano-particles of the invention may be any of the molecules described herein that may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions.

In the context of the present invention, within the meaning of the term ‘equivalent amino acid substitution’ as applied herein, is meant that in certain embodiments one amino acid may be substituted for another within the groups of amino acids indicated herein below:

(i) Amino acids having polar side chains (Asp, Glu, Lys, Arg, His, Asn, GIn, Ser, Thr, Tyr, and Cys); (ii) Amino acids having non-polar side chains (Gly, Ala, Val, Leu, lie, Phe, Trp, Pro, and Met); (iii) Amino acids having aliphatic side chains (Gly, Ala Val, Leu, ile); (iv) Amino acids having cyclic side chains (Phe, Tyr, Trp, His, Pro); (v) Amino acids having aromatic side chains (Phe, Tyr, Trp); (vi) Amino acids having acidic side chains (Asp, Glu); (vii) Amino acids having basic side chains (Lys, Arg, His); (viii) Amino acids having amide side chains (Asn, GIn); (ix) Amino acids having hydroxy side chains (Ser, Thr); (x) Amino acids having sulphur-containing side chains (Cys, Met); (xi) Neutral, weakly hydrophobic amino acids (Pro, Ala, Gly, Ser, Thr); (xii) Hydrophilic, acidic amino acids (GIn, Asn, Glu, Asp), and (xiii) Hydrophobic amino acids (Leu, lie, VaI).

Still further, the activating or inhibitory NK receptors targeted by the targeting moiety of the nanoparticles of the invention may have secondary modifications, such as phosphorylation, acetylation, glycosylation, sulfhydryl bond formation, cleavage and the likes, as long as said modifications retain the functional properties of the original protein, specifically, act as inhibitory or activating receptors. Secondary modifications are often referred to in terms of relative position to certain amino acid residues. For example, a certain sequence positioned carboxyl-terminal to a reference sequence within a polypeptide is located proximal to the carboxyl terminus of the reference sequence, but is not necessarily at the carboxyl terminus of the complete polypeptide.

The invention further encompasses any derivatives, enantiomers, analogues, variants or homologues of any of the NK activating and or inhibitory receptors disclosed herein. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention. Still further, any splice variants of the indicated receptors are also encompassed by the present invention.

In some particular embodiments, the nanoparticles of the invention may be connected and/or associated directly or indirectly in the outer nanoparticle surface thereof with at least one antibody directed against NKp46 or any derivative, splice variant, homolog, ortholog or valiant thereof. In yet some specific embodiments, the nanoparticles of the invention may be associated as a targeting moiety, with any of the anti-NKp46 antibodies disclosed by the Examples section. In yet some further embodiments, the nanoparticle of the invention may be associated with a plurality of antibodies, aptamers or any combinations thereof. It should be noted that each of the plurality of antibodies or aptamers specifically recognizes and binds an NK cell inhibitory or activating receptor of a plurality of inhibitory or activating receptors expressed by NK cells of a subject suffering from an immune-related disorder.

In some embodiments, the target moiety, for example, antibodies and/or aptamers, may be directed against a variety of molecules expressed on a target cell, specifically, any hematopoietic cell, more specifically, NK cell. NK cells within the tumor microenvironment often display a decrease in expression of activating receptors, an increased expression of inhibitory receptors, and a decrease in cytokine secretion and cytotoxic ability. Thus, in some embodiments, the present nanoparticles of the invention may offer personalized therapy, by efficient targeting of the combinations of the invention to the particular NK present in the diseased subject, for example, NK cells obtained from a tumor of a particular patient. Therefore, in yet some further embodiments, the targeting moiety may target receptors, specifically activating and/or inhibitory receptors expressed on an NK cell isolated or obtained from the treated subject or a subject suffering from an immune-related disorder. According to such embodiments, the profiling of the specific molecules (e.g., activating and/or inhibitory receptors) in the patient's NK cells should be determined in order to design specific targeting moieties for the nanoparticles of the invention. More specifically, in some specific and non-limiting embodiments, the nano-particles of the invention may be connected to any commercial antibody specific for any of the inhibitors and/or activating NK cell receptor. Examples for NKp46 antibody may be the LS-C662543 by LSBio. In yet some further embodiments, the nano-particles of the invention may be connected to any commercially available antibody specific for any one of CD57, NKG2A, CD96, the natural cytotoxicity receptors (NCRs) NKp30, NKp44, NKp80, CD16, NKG2D, NKG2C, DNAX Accessory Molecule-1 (DNAM-1), and 2B4, or any of the receptors disclosed by the invention. In some embodiments, the NK cells obtained from the patient may be used for screening antibodies libraries or alternatively, aptamer libraries to design the most appropriate targeting moieties that may be used for a specific patient. As indicated above, in some embodiments, the targeting moieties may be aptamers.

Aptamers are produced by a combinatorial procedure named SELEX (Systematic Evolution of Ligands by Exponential enrichment), that are emerging as promising diagnostic and therapeutic tools. Among selection strategies, procedures using living cells as complex targets (referred as “cell-SELEX”) have been developed as an effective mean to generate aptamers for heavily modified cell surface proteins, assuring the binding of the target in its native conformation. A major advantage of the cell-based procedures is that they may be employed for the targeting of a specific cell type, without any prior knowledge of the specific target, leading to identify multiple aptamers able to recognize specific cell phenotypes and discover new cell biomarkers. To date, aptamers have been developed for different cell types and other complex systems, especially for live cancer cells, and thus, may be also developed for NK cells of a subject suffering from an immune-related disorder. The SELEX procedure involves repeated cycles of: 1. Incubation of the high complexity library with the targets (binding); 2. Removal of unbound sequences and recovery of the bound oligonucleotides (partitioning); 3 Amplification of the bound sequences by PCR (for DNA library) or RT-PCR and transcription (for RNA library). In Whole-Cell SELEX strategy, a fundamental aspect is the inclusion of a counter-selection step, for example, using NK cells obtained from a healthy subject or a subject that is not suffering from the specific immune-related disorder, to avoid the parallel enrichment of aptamers for unwanted targets. The negative selection step is introduced before the positive selection at each round, allowing filtering out sequences against those molecules commonly expressed on both the target and control cell lines. Specifically, an alternative cell-SELEX strategy (referred as “differential cell-SELEX”) has been developed to isolate aptamers able to recognize a specific cell phenotype, rather than a single specific target of interest. This strategy offers the possibility to select multiple ligands discriminating between even closely related cell types, without any prior knowledge of the target. Briefly, the procedure consists of the incubation of the starting library on a non-target cell line (with undesired phenotype, negative selection step) followed by the recovery of unbound oligonucleotides that are, then, incubated on cells with the desired phenotype (positive selection step). Several screening methods have emerged to optimize the selection process, some examples are briefly describe below.

Fluorescence-Activated Cell Sorting (FACS)-SELEX: An extension of the cell-SELEX strategy is a Fluorescence-Activated Cell Sorting (FACS)-based protocol that allows to select aptamers targeting a specific subpopulation. In this strategy, once a fluorescently labeled aptamer library is incubated on target cells, a cell-sorting device is used to differentiate and separate the cell subpopulations that are bound or unbound to the aptamers. Bound aptamers are, then, eluted and amplified. The protocol permits to eliminate dead cell population that, absorbing single-stranded nucleic acid molecules, may negatively influence the selection procedure. In addition, such an approach has two additional advantages. First, it allows the reduction of experimental steps, incorporating in one round both positive and negative selection. Second, it allows to simultaneously monitor the selection process during the rounds without the need of additional binding assays.

Cell Internalization SELEX: Aptamers are emerging as one of the most promising tools for the specific deliver to diseased cells of secondary reagents. Indeed, it has been shown that upon binding to their targets, aptamers can be rapidly internalized, allowing the tissue specific internalization of active therapeutic substances, including nanoparticles, anti-cancer therapeutics, small interfering RNAs (siRNAs), microRNAs, and anti-microRNAs. This permits the exposition to secondary reagents only of target cells, increasing the efficacy and reducing the toxicity of the therapy. Based on these considerations, modified cell-based selection approaches was developed to isolate internalizing aptamers, eliminating those sequences that do not, or very slowly, internalize. In yet some further embodiments, the targeting moieties of the nanoparticles of the invention may be antibodies, each antibody recognizes and binds at least one of a plurality of antigens expressed on the surface of a particular immune-cell (e.g., NK cell) of a patient suffering from an immune-related disorder, or cancer. Thus, in some embodiments, any similarly to the approach discussed above for aptamers, immune-cells of a patient may be used for screening antibody libraries, to select appropriate plurality of antibody targeting moieties for a specific relevant subject. It should be understood that the invention further encompasses the use of any combination of antibodies and aptamers that recognize and target to the plurality of receptors expressed by the patient's immune cell (e.g., NK cell).

The targeting moiety may be connected, linked, conjugated, or associated either directly to the outer surface of the nano-particle of the invention, or any matrix or micellar formulation described herein, or indirectly, for example, via at list one linker. In some embodiments, the at least one targeting moiety is connected (conjugated) to the carrier surface via chemical or physical bonding as described herein below. The association of the at least one targeting moiety with the carrier may be direct or may be via a linker. The linker can be inert, or the linker can have biological activity. The linker must be at minimum bivalent; however, in some embodiments, the linker can be bound to more than one active agent, in which case, the linker is polyvalent. The linker can be composed of any assembly of atoms, including oligomeric and polymeric chains, which functions to connect one or more of the targeting moieties (e.g., antibodies, aptamers), to the nanoparticles. In some cases, the linker may be an oligomeric and polymeric chain, such as an oligo- or polyethylene glycol chain, or an oligo- or poly(amino acid) chain. In some cases the linker is a non-polymeric organic functional group, such as an alkyl group or an alkylaryl group. In certain embodiments, the linker may be hydrophilic to facilitate passage of the nanoparticles across biological membranes. In many cases, the linker is a linear chain; however, in some embodiments, the linker/s may contain one or more branch points. In the case of branched linker, the terminus of each branch point can be functionalized with the targeting moiety. Still further, it should be noted that the targeting moiety may be associated or linked to the nano- or microparticles of the invention either directly or indirectly, for example, via a linker or any adaptor molecule, for example, an adaptor molecule that is based on affinity interactions, for example, avidin-biotin, leucine zipper adaptor and the like. The use of adaptor molecules may enable the use of common or universal nano-particles loaded with the inhibitory combination of the invention (e.g., the siRNAs of the invention), and adapted for personalized treatments by connecting to the affinity adaptor variety of antibodies that are specific for receptors expressed by infiltrating NK cells isolated from the particular patient and profiled by the present invention. In yet some further embodiments, a linker that links the targeting moiety to the nano-particle of the invention may be used. The term “linker” in the context of the invention concerns an amino acid sequence of from about 1 to about 10 or more amino acid residues positioned on the outer layer of the nanoparticles of the invention. The linker is covalently linked or joined to the amino acid residues in its vicinity. For example, a linker in accordance with the invention may be of 1, 2, 3, 4, 5, 6, 7, 8, 9 or 10 or more amino acid residues long. Linkers are often composed of flexible amino acid residues, for example but not limited to glycine and serine so that the adjacent targeting moiety (e.g., an antibody or any other affinity molecule) are free to move relative to one another. In more specific embodiments, the targeting moieties of the nano- or micro-particle or micellar formulation, or vehicle or matrix of the invention, recognize and bind NK cells of a subject suffering from an immune-related disorder, for example, at least one of a cancer, a proliferative disorder, an infectious disease, a graft versus host disease, an inflammatory disorder, an immune-cell mediated disorder and an autoimmune disorder. It should be noted that the immune-related disorders relevant for this aspect are those as described herein after in connection with other aspects of the invention.

Of particular relevance are formulations of the combinations of the invention are combinations encompassed within a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as liposomes or polyelectrolyte multilayer capsules, incorporate nanoparticle (NP) actuators.

More specifically, Controlled drug delivery systems (DDS) have several advantages compared to the traditional forms of drugs. A drug is transported to the place of action, hence, its influence on vital tissues and undesirable side effects can be minimized Accumulation of therapeutic compounds in the target site increases and, consequently, the required doses of drugs are lower. This modern form of therapy is especially important when there is a discrepancy between the dose or the concentration of a drug and its therapeutic results or toxic effects. Cell-specific targeting as described above, can be accomplished by attaching drugs to specially designed carriers. Various nanostructures, including liposomes, polymers, dendrimers, silicon or carbon materials, and magnetic nanoparticles are applicable in the present invention. Polymeric nanoparticles are one technology being developed to enable clinically feasible oral delivery.

The term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid. Examples of lipids forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS). Examples of cationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N-(N′,N′-dimethylaminoethane) carbamoly] cholesterol (DC-Chol); and dimethyl-dioctadecyl ammonium (DDAB), N-[2-[[2,5-bis[3-aminopropyl)amino]-1-oxopentyl]amino]ethyl]-N,N-dimethyl-2,3-bis[(1-oxo-9-octadecenyl)oxy]-1 propanaminium (DOSPA), and ceramide carbamoyl spermine (CCS), or the neutral lipid dioleoylphosphatidyl ethanolamine (DOPE) derivatized with polylysine to form a cationic lipopolymer.

The lipids may be combined with other lipid compatible substances, such as, sterols, lipopolymers etc. A lipopolymer may be a lipid modified by inclusion in its polar headgroup a hydrophilic polymer. The polymer headgroup of a lipopolymer may be preferably water-soluble. In some embodiments, the hydrophilic polymer has a molecular weight equal or above 750 Da. There are numerous polymers which may be attached to lipids to form such lipopolymers, such as, without being limited thereto, polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers. The lipids derivatized into lipopolymers may be neutral, negatively charged, as well as positively charged. The most commonly used and commercially available lipids derivatized into lipopolymers are those based on phosphatidyl ethanolamine (PE), usually, distearoylphosphatidylethanolamine (DSPE).

In some embodiments, the structure forming lipids may be combined with other lipids, such as a sterol. Sterols and in particular cholesterol are known to have an effect on the properties of the lipid's organized structure (lipid assembly), and may be used for stabilization, for affecting surface charge, membrane fluidity. In some embodiments, a sterol, e.g. cholesterol is employed in order to control fluidity of the lipid structure. The greater the ratio sterol:lipids (the structure forming lipids), the more rigid the lipid structure is. Liposomes are often distinguished according to their number of lamellae and size. The liposomes employed in the context of the present disclosure may be multilamellar vesicles (MLVs), multivesicular vesicles (MVVs), small unilamellar vesicles (SUVs), large unilamellar vesicles (LUVs) or large multivesicular vesicles (LMVV). It should be appreciated that the combinations of the invention may be encapsulated or associated with any of the nanostructures described above, specifically, any of the micellar formulations, liposomes, polymers, dendrimers, silicon or carbon materials, polymeric nanoparticles and nanoparticles disclosed herein above. The term “association” may be used interchangeably with the term “entrapped”, “attachment”, “linked”, “embedded”, “absorbed” and the like, and contemplates any manner by which the compounds of the invention is held. This may include for example, physical or chemical attachment to the carrier. Chemical attachment may be via a linker, such as polyethylene glycol. The association provides capturing of the at least one compounds of the invention by the nanostructure such that the release of the at least two compounds used by the invention may be controllable. As indicated above, it should be appreciated that in some embodiments, the nanostructure in accordance with the present disclosure may further comprise at least one targeting moiety on the surface. Such targeting moiety, may facilitate targeting the compound-nanostructures of the invention into a particular target cell, target tissue, target organ or particular cellular organelle target. In some embodiments, that targeting moiety targets the nano-particles to NK cells. It should be noted that the transporting or targeting moiety may be attached directly or indirectly via any linker, and may comprise affinity molecules, for example, antibodies or aptamers or any other affinity molecule, as described herein above, that specifically recognize target antigen on specific hematopoietic cells.

Still further, in some specific and non-limiting embodiments, the nanoparticles of the invention may be composed of phosphatidylcholine (PC), dipalmitoylphosphatidylethanolamine (DPPE), and cholesterol (Chol). In yet some further specific embodiments, the nanoparticles of the invention may comprise PC, DPPE and Chol at molar ratios of 3:1:1 (PC:DPPE:Chol). Still further, the nanoparticles of the invention may be prepared by a lipid-film method as described by the invention.

In yet some further embodiments, the nanoparticles of the invention, also referred to herein as unilamellar nano-scale liposomes (ULNL), are further surface-modified with high molecular weight glycosaminoglycan hyaluronic acid (HA), thereby obtaining HA-NPs. Still further, in some embodiments, the HA-NPs of the invention are coated with monoclonal anti-NKp46 antibody. Natural killer cell p46-related protein (NKp46) is also known as Natural cytotoxicity triggering receptor 1 (NCR1), Lymphocyte antigen 94 homolog (LY94), CD335 Antigen (cluster of differentiation 335, NKP46, NKp46, NK-p46, and CD33. NKp46 is a member of the natural cytotoxicity receptor (NCR) family and was identified as an important regulator of NK cell function. Engagement of the CD335 receptor on NK cells results in increased cellular activation, manifesting as increased cytokine production and release of cytolytic granules. NKp46, as used herein, refers to any one of: the human NKp46 isoform b precursor as denoted by NP_001138929, the isoform c precursor as denoted by NP_001138930.2, isoform e precursor as denoted by NP_001229286, as well as other isoporms as denoted by any one of NP_001229285, NP_001229286 and NP_004820.

In yet some further embodiments, the nanoparticles of the invention may be conjugated or covered by at least one anti-NKp44 antibody. Natural cytotoxicity triggering receptor 2 is a protein that in humans is encoded by the NCR2 gene is a transmembrane glycoprotein characterized by a single extracellular V-type Ig-like domain and a cytoplasmic tail containing an Immunoreceptor Tyrosine-based Inhibitory Motif (ITIM) and no known activating signaling motifs. In some embodiments, the NKp44, as used herein, refers to any one of: the human NKp44 as denoted by any one of NP_001186438, NP_001186439 and NP_004819.

It should be understood that although in some embodiments and aspects of the invention the combinations of the invention are delivered using nanoparticles, the invention further encompasses the option of using any other vector or vehicle for delivery of the combinations of the invention, specifically any combination of inhibitory compounds that inhibit the expression, stability and/or activity of at least one SHP protein and at least two Cbl proteins, that are based on nucleic acid molecules, for example, the siRNAs of the invention, or any oligonucleotide or even any gene editing system as described herein before. In more specific embodiments, such vector may be any one of a viral vector, a non-viral vector and a naked DNA vector.

Vectors, as used herein, are nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, etc., useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as AAV, MMLV, HIV-1, ALV, etc.

Vectors may be provided directly to the subject cells. In other words, the cells are contacted with vectors comprising the oligonucleotides of the invention such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV).

More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

As indicated above, in some embodiments, viral vectors may be applicable in the present invention. The term “viral vector” refers to a replication competent or replication-deficient viral particle which are capable of transferring nucleic acid molecules into a host.

Still further, in some embodiments, the vector may be a naked DNA vector. More specifically, such vector may be for example, a plasmid, minicircle or linear DNA.

Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

In yet some further aspects thereof, the invention provides at least one cell comprising the combinations of the invention and/or any nano-particles comprising the combinations of the invention, specifically as described by the invention, as well as any cell population comprising at least 10% or more of the cells of the invention.

In some specific embodiments, the cell of the invention may comprise any of the combinations disclosed by the invention, specifically, the cell in accordance with some embodiments of the invention may comprise any inhibitory nucleic acid molecules, SMCs, aptamers, peptide, or any combinations thereof, that specifically inhibit at least one of: (i) at least one of SHP-1 and SHP-2; and (ii) at least one of Cbl-b, c-Cbl and Cbl-3.

More specifically, in some embodiments, the combination comprised within the cell of the invention may comprise at least two compounds that comprise at least one nucleic acid molecule. More specifically, each of the nucleic acid molecules is specific for or specifically directed against one of at least one of: (i) at least one of SHP-1 and SHP-2; and (ii) at least one of Cbl-b, c-Cbl and Cbl-3.

In yet some further embodiments, the nucleic acid molecule of the combination comprised within the cell of the invention, may be RNA molecules or any nucleic acid sequence encoding the RNA molecules. In more specific embodiments, such RNA molecules may be at least one of a dsRNA, an antisense RNA, a ssRNA, a gRNA and a Ribozyme.

In certain embodiments, the combinations comprised within the cell of the invention may comprise at least two dsRNA molecules. More specifically, each of the dsRNA molecules may be directed against or specific for one of at least one of: (i) at least one of SHP-1 and SHP-2; and (ii) at least one of Cbl-b, c-Cbl and Cbl-3. In yet more specific embodiments, such dsRNA molecules, may be at least one of siRNA, miRNA, shRNA and piRNAs.

In yet some further embodiments, the combinations comprised within the cell of the invention may comprise at least two of: at least one siRNA molecule specifically directed against, or specific for SHP-1, at least one siRNA molecule specifically directed against Cbl-b, and at least one siRNA molecule specific for, or specifically directed against c-Cbl.

Still further, in some embodiments, the combinations comprised within the cell of the invention may comprise at least one siRNA molecule specifically directed against, or specific for SHP-1, at least one siRNA molecule specifically directed against, or specific for Cbl-b and at least one siRNA molecule specifically directed against or specific for c-Cbl. In yet some further embodiments, of the combination comprised within the cell of the invention, the siRNA molecule specifically directed against, or specific for SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specifically directed against, or specific for Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specifically directed against, or specific for c-Cbl may comprise the nucleic acid sequence as denoted by any one of SEQ ID NO: 5 and SEQ ID NO: 7, or of any variants, homologs or derivatives thereof.

It should be noted that in some embodiments, the cell of the invention may comprise any of the nano- or micro-particle or micellar formulation or vehicle or matrix as disclosed by the invention. In some embodiments, the cell of the invention may be any hematopoietic cell. In yet some further particular embodiments, the cell of the invention may be an NK cell. Still further, in some embodiments, the cell of the invention may be a cell obtained from a subject suffering from an immune related disorder, specifically from a proliferative disorder. As indicated above, the invention further encompasses any population of the cells of the invention. Specifically, such population of cells may be an enriched population of cells comprising the combinations of the invention or any nano-particles thereof, i.e. a population of cells in which a high percentage of cells among the total number of cells in the population of cells comprise the combinations of the invention. Specifically, such a percentage of cells may be 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% of the total population of cells obtained by the invention. In some specific embodiments, at least 50% of the cells of the population may be cells that were manipulated to comprise the combinations of the invention or any nano-particles or any vectors or vehicles thereof.

Another aspect of the invention relates to a pharmaceutical composition comprising as an active ingredient at least one combination comprising at least two compounds that specifically inhibit at least one of, the expression, activity and stability of at least one PTP and at least one E3 ubiquitin-protein ligase. In more specific embodiments, the combinations comprised within the compositions of the invention may comprise at least two compounds that specifically inhibit at least one of, the expression, activity and stability of at least one SHP and at least one member of the Cbl E3 ubiquitin-protein ligase family, or any vehicle, matrix, nano- or micro-particle or micellar formulation comprising the combinations of the invention, or any cell or cell population comprising the combinations of the invention or any nanoparticles of the invention. It should be noted that in some embodiments, the composition of the invention may optionally further comprise at least one of pharmaceutically acceptable carrier/s, excipient/s, auxiliaries, and/or diluent/s.

In yet some further embodiments, the composition of the invention may comprise any of the combinations disclosed by the invention, as well as any of the nanoparticles disclosed by the invention, as well as any cells comprising any of the combinations of the invention or any nanoparticles thereof, as described above in these specific aspects of the invention. More specifically, in some specific embodiments, the compositions of the invention may comprise any of the combinations disclosed by the invention, specifically, the compositions in accordance with some embodiments of the invention may comprise any inhibitory nucleic acid molecules, SMCs, aptamers, peptide, gene editing systems, or any combinations thereof, that specifically inhibit at least one of: (i) at least one of SHP-1 and SHP-2; and (ii) at least one of Cbl-b, c-Cbl and Cbl-3. More specifically, in some embodiments, the combination comprised within the compositions of the invention may comprise at least two compounds that comprise at least one nucleic acid molecule. More specifically, each of the nucleic acid molecules is specific for or specifically directed against one of at least one of: (i) at least one of SHP-1 and SHP-2; and (ii) at least one of Cbl-b, c-Cbl and Cbl-3.

In yet some further embodiments, the nucleic acid molecule of the combination comprised within the compositions of the invention, may be RNA molecules or any nucleic acid sequence encoding the RNA molecules. In more specific embodiments, such RNA molecules may be at least one of a dsRNA, an antisense RNA, a ssRNA and a Ribozyme. In certain embodiments, the combinations comprised within the compositions of the invention may comprise at least two dsRNA molecules. More specifically, each of the dsRNA molecules may be directed against or specific for one of at least one of: (i) at least one of SHP-1 and SHP-2; and (ii) at least one of Cbl-b, c-Cbl and Cbl-3. In yet more specific embodiments, such dsRNA molecules, may be at least one of siRNA, miRNA, shRNA and piRNAs. In yet some further embodiments, the combinations comprised within the compositions of the invention may comprise at least two of: at least one siRNA molecule specifically directed against, or specific for SHP-1, at least one siRNA molecule specifically directed against Cbl-b, and at least one siRNA molecule specific for, or specifically directed against c-Cbl.

Still further, in some embodiments, the combinations comprised within the compositions of the invention may comprise at least one siRNA molecule specifically directed against, or specific for SHP-1, at least one siRNA molecule specifically directed against, or specific for Cbl-b and at least one siRNA molecule specifically directed against or specific for c-Cbl. In yet some further embodiments, of the combination comprised within the compositions of the invention, the siRNA molecule specifically directed against, or specific for SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specifically directed against, or specific for Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specifically directed against, or specific for c-Cbl may comprise the nucleic acid sequence as denoted by any one of SEQ ID NO: 5 and SEQ ID NO: 7, or of any variants, homologs or derivatives thereof.

In some embodiments, the compositions of the invention may comprise any of the nano- or micro-particle or micellar formulation or vehicle or matrix, or any cell or cell population comprising any of the combinations disclosed herein or any nanoparticles thereof as disclosed by the invention. It should be understood that the active ingredients in the combinations of the invention, specifically, the siRNA molecule specific for SHP-1, the siRNA molecule specific for Cbl-b, and the siRNA molecule specific for c-Cbl may be presented in the composition of the invention at any ratio, for example, 1:1:1, 2:2:1, 3:1:1, or between 0.0001 to 10⁶: between 0.0001 to 10⁶: and between 0.0001 to 10⁶. The effective amount of the active ingredients may be in some embodiments, the amount of the compounds sufficient to inhibit the expression, stability and/or activity of any one of the SHP proteins and of the Cbl proteins as discussed above. In some embodiments, the active ingredient in the compositions of the invention may be provided in an amount effective for inhibiting the expression, stability and activity of at least one SHP protein and of the Cbls proteins. Percentage of such inhibitory effect is as indicated herein before in connection with the combinations of the invention. Such inhibitory effect is in extent for activating hematopoietic cells, specifically NK cells in an inhibitory immunological synapse.

In yet some other specific embodiments the effective amount of the combinations of the invention, specifically, the siRNA molecule specific for SHP-1, the siRNA molecule specific for Cbl-b, and the siRNA molecule specific for c-Cbl may range between 0.001 mg to 10,000 mg, specifically, 10, 20, 30, 40, 50, 60, 70, 80, 90, 100, 200, 300, 400, 500, 600, 700, 800, 900, 1000, 2000, 3000, 4000, 5000, 6000, 7000, 8000, 8000, 9000, 10000 mg a day or more. In yet some further embodiments, the effective amount of the combinations of the invention, specifically, the siRNA molecule specific for SHP-1, the siRNA molecule specific for Cbl-b, and the siRNA molecule specific for c-Cbl may range between about 0.1 to 100 microgram/kg, specifically, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60, 70, 80, 90, 100 microgram/kg and more. In some specific embodiments, an effective amount for in vitro treatment may be about 1 to 10 micrograms, specifically, 6.6 micrograms. Still further, in some embodiments the effective amount for in vivo treatment may range between about 1 to 50 microgram/kg, specifically, about 20 microgram/kg. The pharmaceutical compositions of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ, etc. More specifically, the combinations of the invention or any nanoparticles or compositions thereof, described herein after, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). It should be noted that any of the administration modes discussed herein, may be applicable for any of the methods of the invention as described in further aspects of the invention herein after.

Compositions and formulations for oral administration may include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable. Pharmaceutical formulations adapted for rectal administration may be presented as suppositories or enemas. Pharmaceutical formulations adapted for vaginal administration may be presented as pessaries, tampons, creams, gels, pastes, foams or spray formulations.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question, for example those suitable for oral administration may include flavoring agents.

The compositions of the invention may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis. Formulations for ocular and aural administration may be formulated to be immediate and/or modified release. Modified release includes delayed, sustained, pulsed, controlled, targeted, and programmed release.

In specific embodiments, the unit dosage formulations are those containing a daily dose or sub-dose, as herein above recited, or an appropriate fraction thereof, of an active ingredient.

In yet a further aspect, the invention relates to a method for activating at least one hematopoietic cells in an inhibitory immunological synapse (IS). In yet some further specific embodiments, the methods of the invention may be suitable for activating at least one of NK cells, T cells and B cells in an inhibitory immunological synapse. Still further, the methods of the invention may be particularly suitable for activating NK cells in an inhibitory immunological synapse (NKIS). In more specific embodiments, the method may comprise the step of contacting the NK cell with an activating effective amount of at least one combination comprising at least two compounds that specifically inhibit at least one of, the expression, activity and stability of at least one PTP, specifically, SHP protein and of at least one E3 ubiquitin-protein ligase, specifically, at least one member of the Cbl family, or any vehicle, matrix, nano- or micro-particle, micellar formulation or composition comprising the combination of the invention.

The methods of the invention activate cells in an inhibitory immunological synapse. The term “immunological synapse” refers to an interface between an antigen-presenting cell (APC), a target cell, or both and a lymphocyte such as a Natural Killer (NK) cell, an effector T cell, or even a B cell. It is thus meant that using the compounds according to the above can shift the balance between activating and inhibitory stimuli to which said lymphocyte is subjected at IS. The IS model was originally denoted the interaction between a T helper cell and APC, but may also apply to the NK cell IS (NKIS). Certain aspects are particularly relevant to NK cells, such as directed secretion of lytic granules for cytotoxicity. This model when applied to NK cell activation is especially informative for the inhibitory NKIS, which a striking example wherein inhibition of signaling leaves the synapse in its nascent, inverted state (early stage).

The methods of the invention are thus applicable in some embodiments to a lymphocyte cell that is a NK cell forming an inhibitory NK immunological synapse (NKIS). More specifically, Natural killer cells or NK cells are a type of cytotoxic lymphocyte critical to the innate immune system. NK cells (belonging to the group of innate lymphoid cells) are defined as large granular lymphocytes (LGL). The role NK cells play is analogous to that of cytotoxic T cells in the vertebrate adaptive immune response. NK cells provide rapid responses to viral-infected cells, acting at around three days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. Still further, in a broader sense, “NKIS” denotes the dynamic interface formed between an NK cell and its target cell. Formation of NKIS involves several distinct stages, beginning with the initiation of contact with a target cell and culminating in the directed delivery of lytic granule contents to lyse the target cell. Progression through the individual stages is methodical and underlies the precision with which NK cells select and kill susceptible target cells (including virally infected cells and cancerous cells) that they encounter during their routine surveillance of the body.

More specifically, the formation of a mature and functional NKIS can be divided into a series of sequential (nonparallel) stages: the recognition and initiation stage, the effector stage and the termination stage. Together, these processes enable the delivery of lytic granules to the synapse followed by their close association with the NK cell membrane to which they can fuse and release their contents onto the target cell. Because lytic granules exist in resting NK cells before activation, each stage must be controlled to prevent accidental release of cytotoxic mediators and to enable rapid directed secretion at the appropriate moment. Of particular relevance are molecules related to the above processes, which can be used as markers for evaluating activity of the combinations of the present invention and any nano-particles thereof. Specifically: the initial stage is characterized by formation of a close association between the NK cell and a target cell, initial signaling and adherence of NK cell to its target cell. This stage is facilitated by a number of molecules, including, although not limited to, members of the selectin family, the CD2 receptor, and receptors from the integrin family of adhesion molecules in particular such as the integrins lymphocyte function-associated antigen 1 (LFA1; CD11a/CD18) and MAC1 (CD11b/CD18). Importantly, this initial stage is rapid and occurs before molecular patterning is evident. The decision whether NK cell progresses to maturation and molecular reorganization at NKIS depends on the level of signals through inhibitory receptors (KIRs, Killer-cell Immunoglobulin-like Receptors), which can establish a so-called inhibitory synapse. Such regulation ensures that NK cells effectively carry out their surveillance function, by leaving most cells undisturbed, while being poised to destroy those that are diseased. The inhibitory NKIS is especially elegant in that it directly interferes with the ability of the lytic synapse to progress past the initiation stage. The effector stage is characterized by a number of processes, most prominent of which are (1) formation of a stable NK cell—target cell interface with a ‘cleft’ into which cytolytic molecules are secreted; (2) recruitment of lytic granules to the synapse; (3) clearance of a conduit in the NK cell cortex through which lytic granules could be directed to the cell membrane; and (4) fusion of the lytic-granule membrane with plasma membrane for release of lytic-granule contents. Parallel events include receptor clustering, lipid-raft aggregation, further activation signaling and lytic-granule redistribution. Among receptors that undergo clustering, the most important for both, adhesion and triggering of cytotoxicity, are CD11a, CD1 lb and CD2. Another requirement for effector function is polarization of lytic granules to NKIS, or in other words movement of the granules along the microtubules to the microtubule-organizing centre (MTOC). Signals required for MTOC polarization include ERK (extracellular-signal-regulated kinase) phosphorylation, VAV1 activation and PYK2 (protein tyrosine kinase 2) activities. Still further, granule docking to the synapse requires members of the RAB family of small GTPases, which are important regulators of vesicle trafficking and compartmentalization. RAB27a also performs this function in docking lytic granules in CTLs. Of further relevance are Munc13-4 (putative vesicle priming factor) and SNAREs (N-ethylmaleimide—sensitive fusion protein attachment protein receptors) and their regulators acting in a coordinated manner to facilitate membrane fusion and providing a fine-tuning of NKIS; SHP-1 (a SH2 domain containing tyrosine phosphatase), yet another regulator of the effector function.

Termination stages of NKIS refer to those that occur after the lytic-granule contents have been secreted. Those include a period of inactivity and down modulation of the accumulated activating receptors followed by NK cell detachment from the target cell and recycling of cytolytic capacity. Once NK cell has carried out its cytolytic function, it can detach from the target cell and restore its ability to kill another susceptible cell. At the inhibitory synapse, detachment may result from reduced integrity of interactions between the F-actin cortex and the plasma membrane through dephosphorylation of ERM protein targets. The signals initiating the process of recycling NK cytolytic capacity are largely unknown, apart from activation of the nuclear factor-κB (NF-κB) which has been shown to serve as a transcription factor for expression of the lytic granule component perforin. As mentioned above, secretion of cytolytic granules in response to increased intracellular Ca²⁺ flux is characteristic of ‘Termination stage’ of a lymphocyte responding to an activating stimulus at IS, a which is common to NK cells and CTLs, i.e. innate or adaptive immune response. When CTL or NK cells kill infected or cancerous cells they secrete cytolytic proteins (perforin and granzymes) into the target cell. These “death factors” are pre-stored in cytolytic granules within the CTL until an increase in the intracellular Ca2⁺ drives granule to exocytosis. Secretion of cytolytic granules and increased intracellular Ca²⁺ flux are measurable and can serve as markers for evaluating the activity of the presently conceived compounds.

In some embodiments, the lymphocyte cell modulated, specifically activated by the combinations of the invention, or any nano-particles thereof, may be at least one of an NK cell, a T cell and a B cell forming an inhibitory IS, specifically, NK cells.

In yet some further embodiments, the at least two compounds in the combination used by the methods of the invention may comprise at least one nucleic acid molecule, each nucleic acid molecule is specifically directed against one of at least one of: (i) at least one of SHP-1 and SHP-2; and (ii) at least one of Cbl-b, c-Cbl and Cbl-3.

In some embodiments, such compound may be alternatively, or additionally, any SMCs, aptamer, peptide, antibody or any combinations thereof.

In some specific embodiments, the compounds in the combination used by the methods of the invention may be at least two nucleic acid molecules. In yet some further embodiments, such nucleic acid molecule may be an RNA molecule or any nucleic acid sequence encoding said RNA molecule. In some further embodiments, the RNA molecule may be at least one of a dsRNA, an antisense RNA, a ssRNA, a Ribozyme and a guide RNA.

In more specific embodiments, the dsRNA may be at least one of siRNA, miRNA, shRNA and piRNAs, as discussed herein above in other aspects of the invention.

In yet some further embodiments, the combination used by the methods of the invention may comprise at least two of: at least one siRNA molecule specific for, or specifically directed against SHP-1, at least one siRNA molecule specific for, or specifically directed against Cbl-b, and at least one siRNA molecule specific for, or specifically directed against c-Cbl.

Still further, in some embodiments, the combination used by the methods of the invention may comprise at least one siRNA molecule specific for, or specifically directed against SHP-1, at least one siRNA molecule specific for, or specifically directed against Cbl-b and at least one siRNA molecule specific for, or specifically directed against c-Cbl.

In more specific embodiments of the methods of the invention, the siRNA molecule specific for, or specifically directed against SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specific for, or specifically directed against Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specific for, or specifically directed against c-Cbl may comprise the nucleic acid sequence as denoted by any one of SEQ ID NO: 5 and SEQ ID NO: 7, or of any variants, homologs or derivatives thereof.

In yet some further embodiments, the combinations used by the methods of the invention may be comprised within at least one of a vehicle, matrix, nano- or micro-particle, a micellar formulation or a composition In more specific embodiments, the combinations used by the methods of the invention may be comprised within at least one nanoparticle, specifically, any of the nanoparticles disclosed by the invention.

In some further embodiments, at least one targeting moiety may be associated directly or indirectly with the outer nanoparticle surface of the nanoparticle, micro-particle, micellar formulation, vehicle or matrix used by the methods of the invention.

In yet some further embodiments, the targeting moiety of the nanoparticle used by the methods of the invention may be at least one of an antibody, an aptamer, a ligand or any combinations thereof, that specifically recognizes and binds at least one molecule expressed on the surface of at least one hematopoietic cell.

In yet some further embodiments, the hematopoietic cell recognized by the targeting moiety of the nano- or micro-particle or micellar formulation or vehicle or matrix used by the methods of the invention, may be at least one NK cell.

In yet some further embodiments, the nanoparticle used by the methods of the invention may be associated directly or indirectly by the outer nanoparticle surface thereof with at least one of: at least one antibody, at least one aptamer or any combinations thereof, that specifically recognize and bind at least one of: (i) at least one NK cell activating receptor; and (ii) at least one NK cell inhibitory receptor; or any combinations thereof.

In some specific embodiments, the NK cell activating receptor may be at least one of NKp46, NKp44, NKp30, NKp80, CD27, LFA-1, CD16, NKG2D, CRTAM, DNAM-1, 2B4 (CD244), and any derivatives and splice variants thereof. In yet some further embodiments, the NK cell inhibitory receptor may be at least one of KIR, PD-1, Ly49, NKRP1A, CD94, NKG2A, TIGIT, CD96, TIM-3, LAG-3, CEACAM1, LAIR-1, LILRB1, BTLA, CTLA4, 2B4, and any derivatives, splice variants, homologs and orthologs thereof or any combinations thereof.

In some embodiments of the methods of the invention, the hematopoietic cell is a T cell. Thus, the nano- or micro-particle, micellar formulation, vehicle or matrix used by the methods of the invention is connected directly or indirectly by the outer nanoparticle surface thereof with at least one antibody, at least one aptamer or any combinations thereof, that specifically recognize and bind at least one of at least one T cell activating receptor and at least one T cell inhibitory receptor or any combinations thereof. In some embodiments, the T cell activating receptor is any one of CD3, CD28, CD69, CD4, CD8, CD137, and T cell inhibitory receptor is at least one of PD-1, BTLA, CD160, 2B4, CTLA-4, LAG-3, Tigit, CECAM, Tim3, Lairl, PirB, PECAM1, CD22 (Siglec 2), Siglec 7, Siglec 9, KLRG1, ILT2, KIR2DL/3DL, CD72, CD94, NKG2A and CD5.

In yet some further embodiments of the methods of the invention, the nanoparticle of the invention may be associated with a plurality of antibodies, aptamers, ligands, or any combinations thereof. It should be noted that each of the plurality of antibodies or aptamers specifically recognizes and binds an NK cell inhibitory or activating receptor of a plurality of inhibitory or activating receptors expressed by NK cells of a subject suffering from an immune-related disorder.

In some embodiments, subject is suffering from an immune-related disorder, for example, at least one of a cancer, a proliferative disorder, an infectious disease, a graft versus host disease, an inflammatory disorder, an immune-cell mediated disorder and an autoimmune disorder.

Still further, in some embodiments, the methods of the invention are specifically applicable for activating NK cell in a subject suffering from an immune-related disorder. It should be noted that all the immune-related disorders applicable in the present aspect are those disclosed by the invention herein after, in connection with other aspects of the invention.

In yet a further aspect, the invention relates to a method for treating, preventing, inhibiting, reducing, eliminating, protecting or delaying the onset of an immune-related disorder in a subject in need thereof. More specifically, the method of the invention may comprise the step of administering to the treated subject a therapeutically effective amount of at least one combination comprising at least two compounds that specifically inhibit at least one of, the expression, activity and stability of at least one PTP, specifically, SHP protein and of at least one E3 ubiquitin-protein ligase, specifically, at least one member of the Cbl family, or any nano- or micro-particle, micellar formulation or any vehicle or matrix comprising the combination of the invention or any cell comprising the combinations of the invention or an nanoparticles thereof, or composition comprising the combination of the invention, any nanoparticles thereof or any cells comprising the combinations or nanoparticles thereof.

In yet some further embodiments, the at least two compounds in the combination used by the methods of the invention may be at least one nucleic acid molecule, each nucleic acid molecule is specifically directed against at least one of: (i) at least one of SHP-1 and SHP-2; and (ii) at least one of Cbl-b, c-Cbl and Cbl-3.

In some embodiments, such compound may be alternatively, any small molecule, aptamer, peptide, antibody or any combinations thereof. Such compounds inhibit the expression, stability and/or activity of at least one SHP protein and at least one of Cbl-b, c-Cbl and Cbl-3.

As indicated above, in some specific embodiments, the compounds in the combination used by the methods of the invention may be at least two nucleic acid molecules. In yet some further embodiments, such nucleic acid molecule may be an RNA molecule or any nucleic acid sequence encoding said RNA molecule. In some further embodiments, the RNA molecule may be at least one of a dsRNA, an antisense RNA, a ssRNA and a Ribozyme.

In more specific embodiments, the dsRNA may be at least one of siRNA, miRNA, shRNA and piRNAs.

In yet some further embodiments, the combination used by the methods of the invention may comprise at least two of: at least one siRNA molecule specific for, or specifically directed against SHP-1, at least one siRNA molecule specific for, or specifically directed against Cbl-b, and at least one siRNA molecule specific for, or specifically directed against c-Cbl.

Still further, in some embodiments, the combination used by the methods of the invention may comprise at least one siRNA molecule specific for, or specifically directed against SHP-1, at least one siRNA molecule specific for, or specifically directed against Cbl-b and at least one siRNA molecule specific for, or specifically directed against c-Cbl.

In more specific embodiments of the methods of the invention, the siRNA molecule specific for, or specifically directed against SHP-1 may comprise the nucleic acid sequence as denoted by any one of SEQ ID NO: 9 and SEQ ID NO: 11, or of any variants, homologs or derivatives thereof. In yet some further embodiments, the siRNA molecule specific for, or specifically directed against Cbl-b, may comprise the nucleic acid sequence as denoted by any one of SEQ ID NO: 1 and SEQ ID NO: 3, or of any variants, homologs or derivatives thereof. Still further, in some embodiments, the siRNA molecule specific for, or specifically directed against c-Cbl may comprise the nucleic acid sequence as denoted by any one of SEQ ID NO: 5 and SEQ ID NO: 7, or of any variants, homologs or derivatives thereof.

In yet some further embodiments, the combinations used by the methods of the invention may be comprised within at least one of a nano- or micro-particle, a micellar formulation, a vehicle or a matrix, or a cell or a composition comprising any of the above. In some further embodiments, at least one targeting moiety may be associated directly or indirectly with the outer nanoparticle surface of the nanoparticle used by the methods of the invention. In yet some further embodiments, the targeting moiety of the nanoparticle used by the methods of the invention may be at least one of an antibody, an aptamer, a ligand or any combinations thereof, that specifically recognizes and binds at least one molecule expressed on the surface of at least one hematopoietic cell. In yet some further embodiments, the hematopoietic cell recognized by the targeting moiety of the vehicle, matrix, nano- or micro-particle or micellar formulation used by the methods of the invention, may be any one of NK cells, T cells B cells and any combinations thereof, of the treated subject. In yet some further specific embodiments, such hematopoietic cells may be at least one NK cell of the treated subject. In yet some further specific embodiments, in case the subject is suffering from cancer, the NK cell may be at least one tumor infiltrating natural killer cell. As used herein, tumor infiltrating Natural Killer (NK) lymphocyte cells, are NK cells infiltrated, and penetrated to the tumor tissue. The presence of tumor infiltrating NK may indicate an ongoing immune response toward the tumor. In yet some further embodiments, the nanoparticle used by the methods of the invention may be associated directly or indirectly by the outer nanoparticle surface thereof with at least one of: at least one antibody, at least one aptamer or any combinations thereof, that specifically recognize and bind at least one of: (i) at least one NK cell activating receptor; and (ii) at least one NK cell inhibitory receptor; or any combinations thereof. In some specific embodiments, the NK cell activating receptor may be at least one of NKp46, NKp44, NKp30, CD27, LFA-1, CD16, NKG2D, CRTAM, DNAM-1, 2B4 and any derivatives and splice variants thereof. In yet some further embodiments, the NK cell inhibitory receptor may be at least one of KIR, PD-1, Ly49, NKRP1A, CD94, NKG2A, TIGIT, CD96, TIM-3, LAG-3, CEACAM1, CTLA4, LAIR-1, LILRB1, and any derivatives, splice variants, homologs and orthologs thereof or any combinations thereof. In yet some further embodiments, the nanoparticle used by the methods of the invention may be associated with a plurality of antibodies, aptamers or any combinations thereof, wherein each of said plurality of antibodies or aptamers specifically recognizes and binds an NK cell inhibitory or activating receptor of a plurality of inhibitory or activating receptors expressed by NK cells of the subject to be treated. In yet some further embodiments, the treated subject is a subject suffering from an immune-related disorder. In more specific embodiments, the immune-related disorder may be at least one of a viral infection, cancer, a proliferative disorder, a graft versus host disease, an inflammatory disorder, an immune-cell mediated disorder and an autoimmune disorder.

As noted above, the methods of the invention may be relevant for treating any immune-related disorder, for example, an infectious disease, specifically, a viral infection, cancer or any other proliferative disorder, a graft versus host disease, an inflammatory disorder, an immune-cell mediated disorder and an autoimmune disorder. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. More specifically, an ‘immune-related disorder’, as meant herein, encompasses a range of dysfunctions of the innate and adaptive immune systems. In more specific terms, immune-related disorder can be characterized, for example, (1) by the component(s) of the immune system; (2) by whether the immune system is overactive or underactive; (3) by whether the condition is congenital or acquired, as will be specified herein after.

In some specific embodiments, the methods of the invention may be used for treating cancer or any other proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors.

Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered. Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, any neurological cancer, specifically, neuroblastoma, astrocytoma, neuronal lymphoma and the like, vascular system, hemangiosarcoma and Kaposi's sarcoma.

In some embodiments, the method of the invention may be used to treat a proliferative disorder, cancer, tumor and malignancy by activating/enhancing antitumor immunity. The term “antitumor immunity” refers to innate and adaptive immune responses which may lead to tumor control.

The immune system can be activated by tumor antigens and, once primed, can elicit an antitumor response. Natural Killer (NK) cells are a front-line defense against drug-resistant tumors and can provide tumoricidal activity to enhance tumor immune surveillance. Cytokines like IFN-γ or TNF play a crucial role in creating an immunogenic microenvironment and therefore are key players in the fight against metastatic cancer. Critical aspects in the tumor—immune system interface include the processing and presentation of released antigens by antigen-presenting cells (APCs), interaction with T lymphocytes, subsequent immune/T-cell activation, trafficking of antigen-specific effector cells, and, ultimately, the engagement of the target tumor cell by the activated effector T cell.

Nevertheless, although often successful in preventing tumor outgrowth, this “cancer-immunity cycle” can be disrupted by artifices involved in immune escape and development of tolerance, culminating with the evasion and proliferation of malignant cells. Furthermore, the tumor microenvironment induces suppression and reduced activity of NK and T cells, through the secretion of inhibitory factors suppressing the anti-tumor response, a phenomena known as exhaustion. Using the combinations of the invention or any nanoparticles or compositions thereof, provides methods and compositions for activation of lymphocytes, specifically, NK cells for enhancing anti-tumor immunity.

In yet other embodiments, the methods, as well as the combinations, nanoparticles, compositions and kits of the invention may be also applicable for treating a subject suffering from an infectious disease. More specifically, such infectious disease may be any one of viral diseases, protozoan diseases, bacterial diseases, parasitic diseases, fungal diseases and mycoplasma diseases.

It should be appreciated that an infectious disease as used herein also encompasses any infectious disease caused by a pathogenic agent. Pathogenic agents include viruses, prokaryotic microorganisms, lower eukaryotic microorganisms, complex eukaryotic organisms, fungi, prions, parasites, yeasts, toxins and venoms.

Of particular relevance are infectious diseases caused by a bacterial pathogen. A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Pseudomonas sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

In yet some further relevant diseases are infectious diseases caused by a viral pathogen. The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, coronaviruses, retroviruses, zika virus, ebola virus, hepatitis A, poliovirus, rubella virus, hepatitis C, arboviruses, rabies virus, influenza viruses A and B, measles virus, mumps virus, HIV, HTLV I and II.

The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

The term “parasite” includes, but not limited to, infections caused by somatic tapeworms, blood flukes, tissue roundworms, ameba, and Plasmodium, Trypanosoma, Leishmania, and Toxoplasma species.

Still further, in certain embodiments, the methods, as well as the combinations, nanoparticles, compositions and kits and compositions of the invention may be applicable for treating disorders associated with immunodeficiency.

In some specific embodiments wherein the immune-related disorder or condition may be a primary or a secondary immunodeficiency. It should be understood that any of the immune-related disorders described herein after in connection with other aspects of the invention are also applicable or the present aspect as well.

‘Immunodeficiency’, primary or secondary, meaning inherited or acquired, respectively. The term ‘immunodeficiency’ is intended to convey a state of an organism, wherein the immune system's ability for immuno-surveillance of infectious disease or cancer is compromised or entirely absent.

According to the International Union of Immunological Societies, more than 150 primary immunodeficiency diseases (PIDs) have been characterized, and the number of acquired (or secondary) immuno-deficiencies exceeds the number of PIDs. PIDs are those caused by inherited genetic mutations. Secondary immuno-deficiencies are caused by various conditions, aging or agents such as viruses or immune suppressing drugs. A number of notable examples of PIDs include Severe combined immunodeficiency (SCID), DiGeorge syndrome, Hyperimmunoglobulin E syndrome (also known as Job's Syndrome), Common variable immunodeficiency (CVID): B-cell levels are normal in circulation but with decreased production of IgG throughout the years, so it is the only primary immune disorder that presents onset in the late teens. Chronic granulomatous disease (CGD): a deficiency in NADPH oxidase enzyme, which causes failure to generate oxygen radicals. Classical recurrent infection from catalase positive bacteria and fungi. Wiskott-Aldrich syndrome (WAS); autoimmune lymphoproliferative syndrome (ALPS); Hyper IgM syndrome: X-linked disorder that causes a deficiency in the production of CD40 ligand on activated T-cells. This increases the production and release of IgM into circulation. The B-cell and T-cell numbers are within normal limits. Increased susceptibility to extracellular bacteria and opportunistic infections. Leukocyte adhesion deficiency (LAD); NF-κB Essential Modifier (NEMO) Mutations; Selective immunoglobulin A deficiency: the most common defect of the humoral immunity, characterized by a deficiency of IgA. Produces repeating sino-pulmonary and gastrointestinal infections. X-linked agammaglobulinemia (XLA; also known as Bruton type agammaglobulinemia): characterized by a deficiency in tyrosine kinase enzyme that blocks B-cell maturation in the bone marrow. No B-cells are produced to circulation and thus, there are no immunoglobulin classes, although there tends to be a normal cell-mediated immunity. X-linked lymphoproliferative disease (XLP); and Ataxia-telangiectasia. Thus patients' populations diagnosed with one of PIDs can particularly benefit from methods and compositions of the compounds according to the present invention.

With respect to secondary immunodeficiencies, those can be manifested in both the young and the elderly. Under normal conditions immune responses are beginning to decline at around 50 years of age, what is called immunosenescence. The term ‘immunosenescence’ refers to the gradual deterioration of the immune system brought on by natural age advancement. It involves both the host's capacity to respond to infections and the development of long-term immune memory. Additional common causes of secondary immunodeficiency include severe burns, malnutrition, certain types of cancer, and chemotherapy in cancer patients.

More specifically, in developed countries, obesity, alcoholism, and drug use are common causes of poor immune function. However, malnutrition is the most common cause of immunodeficiency in developing countries. Diets lacking sufficient protein are associated with impaired cell-mediated immunity, complement activity, phagocyte function, IgA antibody concentrations, and cytokine production. Additionally, the loss of the thymus at an early age through surgical removal, for example, results in severe immunodeficiency and high susceptibility to infections.

Of particular relevance to the present context are cellular immunodeficiencies associated with cancer and certain viral pathogens. A cellular immunodeficiency refers to a deficiency the count or function of T lymphocytes, which are the main type of cells responsible for the cellular adaptive immune response in attacking viruses, cancer cells and other parasites. Extensive research has reasonably well established the role of immunodeficiency in cancers of the head and neck, lung, esophagus and breast. Among virally induced immunodeficiencies, the most notable example is AIDS (Acquired Immunodeficiency Syndrome) cause by HIV infection. The role of HIV as a direct cause of cellular immunodeficiency, particularly the deficiency of the CD4+ T helper lymphocyte population, has been well established. Additional examples of viral- or pathogen-induced immunodeficiencies include, although not limited to chickenpox, cytomegalovirus, German measles, measles, tuberculosis, infectious mononucleosis (Epstein-Barr virus), chronic hepatitis, lupus, and bacterial and fungal infections. One of the most recent examples is virus-induced Severe Acute Respiratory Syndrome (SARS). These and additional examples of disorders related to cellular immunodeficiency may include Aplastic anemia, Leukemia, Multiple myeloma, Sickle cell disease, chromosomal disorders such as Down syndrome, infectious diseases caused by pathogens such as Cytomegalovirus, Epstein-Barr virus, Human immunodeficiency virus (HIV), Measles and certain bacterial infections. Chronic kidney disease, Nephrotic syndrome, Hepatitis, Liver failure and other conditions caused by Malnutrition, alcoholism and burns.

Thus patients' populations diagnosed with one of the secondary immunodeficiencies, and particularly one of the cellular immunodeficiencies as above, can particularly benefit from methods, as well as the combinations, nanoparticles, cells, compositions and kits of the present invention. Differential diagnosis of such immunodeficient patients is routinely performed in various clinical settings.

Additional secondary immunodeficiencies may result following bone marrow (BM) transplantation, gene therapy or adaptive cell transfer.

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin). Performance of this medical procedure usually requires the destruction of the recipient's immune system using radiation or chemotherapy before the transplantation. To limit the risks of transplanted stem cell rejection or of severe graft-versus-host disease in allogeneic HSCT, the donor should preferably have the same human leukocyte antigens (HLA) as the recipient. In the case of a bone marrow transplant, the HSC are removed from a large bone of the donor, typically the pelvis, through a large needle that reaches the center of the bone. Peripheral blood stem cells are now the most common source of stem cells for HSCT. They are collected from the blood through a process known as apheresis. The donor's blood is withdrawn through a sterile needle in one arm and passed through a machine that removes white blood cells. The red blood cells are returned to the donor. The peripheral stem cell yield is boosted with daily subcutaneous injections of Granulocyte-colony stimulating factor (G-CSF), serving to mobilize stem cells from the donor's bone marrow into the peripheral circulation. It should be noted that amniotic fluid as well as umbilical cord blood may be also used as a source of stem cells for HSCT.

Gene therapy is the therapeutic delivery of nucleic acid polymers into a patient's cells as a drug to treat disease. Gene therapy is a way to fix a genetic problem at its source. The polymers are either translated into proteins, interfere with target gene expression, or possibly correct genetic mutations. The most common form uses DNA that encodes a functional, therapeutic gene to replace a mutated gene. The polymer molecule is packaged within a “vector”, which carries the molecule inside cells.

Adaptive cell transfer (ACT) is the transfer of cells into a patient. The cells may have originated from the patient or from another individual. The cells are most commonly derived from the immune system, with the goal of improving immune functionality and characteristics. In cancer immunotherapy, T cells are extracted from the patient, genetically modified and cultured in vitro and returned to the same patient.

Of further relevance are patients' populations diagnosed with stress-induced immune-related diseases. Many studies have shown that exposure to physical or psychological stress can affect disease outcomes in immune-related disorders such as viral and bacterial infections, contact dermatitis and allergy. Stress, being it acute and short, or chronic and persistent have been shown to influence and modify various components of the immune system, in particular stress has be related to leukocytosis, increased NK cell cytotoxicity and reduced proliferative response to mitogens.

Of further relevance are patients' populations diagnosed with one of hypersensitivities of an immune response or allergies. Hypersensitivities are divided into four classes (Type I-IV) based on the mechanisms involved and the time course of the hypersensitive reaction. Type I is an immediate or anaphylactic reaction, often associated with allergy; it is mediated by IgE antibodies that trigger degranulation of mast cells and basophils. Type II (also called antibody-dependent or cytotoxic) occurs when antibodies bind to antigens on the patient's own cells, marking them for destruction; it is mediated by IgG and IgM antibodies. Type III and Type IV (also known as cell-mediated or delayed type) are mediated by T cells, monocytes, and macrophages; Type IV reactions are involved in many autoimmune and infectious diseases. A partial list including the most common allergies includes but not limited to Seasonal allergy, Mastocytosis, Perennial allergy, Anaphylaxis, Food allergy, Allergic rhinitis and Atopic dermatitis.

Of further relevance are patients diagnosed with splenomegaly (enlargement of spleen) or hypersplenism. Splenomegaly of between 11-20 cm greater than 20 cm in the size of spleen has been associated with hemolytic anemias, and other diseases involving abnormal red blood cells being destroyed in the spleen, as well as with other disorders, including congestion due to portal hypertension, and infiltration by leukemias and lymphomas.

In further specific embodiments, compositions and methods of the present invention can be applied to prevent an immunodeficiency and/or GvHD in immunocomprimised cancer patients, being it a result of cancer itself (as mentioned above) or an adverse effect of high doses of chemotherapy or radiotherapy (which may induce burns).

A number of human diseases were specifically related to NK cell deficiency and deficient NKIS. Those include certain PIDs characterized by genetic aberrations that impair NK cells function. Several of these diseases induce a specific blockade in the stages leading to the formation of a functional lytic synapse. Most of these diseases can result in haemophagocytic lymphohistiocytosis (HLH), i.e. an inappropriately robust immune response to infection (typically with herpesviruses), which results in a persistent systemic inflammatory syndrome. This leads to the physiological symptoms of septic shock, but is also associated with the pathological finding of haematophagocytosis (the ingestion of red blood cells by phagocytes). The NK cells are most relevant to the HLH phenotype, given their localization to marginal zones in lymphoid organs after viral infection, their innate function early in the course of infection and their inherent ability to eliminate hyperactivated macrophages.

It is thus meant that the combinations of the invention or any nanoparticles or compositions or methods thereof, are particularly applicable to patients diagnosed with one of the disorders related to NK cell or NKIS deficiency, or abnormal NK lytic granule trafficking. Notable examples of disorders belonging to this group are detailed below.

Leukocyte adhesion deficiency type I (LAD-I) results from a defect in the CD18 (β-integrin) component of leukocyte integrin heterodimers. Thus, LAD-I leukocytes do not appropriately adhere to inflamed or activated cells and cannot localize effectively to tissues and sites of inflammation. This leads to increased numbers of leukocytes in the blood and susceptibility to infectious diseases. Because early steps in NK-cell synapse formation—adhesion and activation signaling depend on integrins, NK cells from patients with LAD-I do not adhere to their target cells, resulting in defective cytotoxicity. LAD-I is also distinguished from other diseases discussed here because it does not lead to HLH.

Wiskott-Aldrich syndrome (WAS) results from a hematopoietic-cell-specific defect in actin reorganization and cell signaling due to WASP deficiency. Patients lacking WASP expression or expressing abnormal WASP have NK cells with decreased cytolytic capacity. Clinically, patients with WAS are susceptible to herpesviruse and can develop HLH, thereby demonstrating the functional relevance of WASP deficiency for the NK-cell lytic synapse. Formation of the lytic synapse is abnormal in NK cells from WAS patients and includes decreased F-actin accumulation and adhesion-receptor clustering at the synapse.

Chediak-Higashi syndrome (CHS) and Hermansky-Pudlak syndrome type II (HPS2) both affect the normal formation of lytic granules and lead to the presence of “giant” lytic granules. Both are also associated with albinism, which is caused by aberrant functioning of melanocytes, which pigment skin via secretion of melanosomes (an equivalent of lytic granules). CHS and HPS2 are similar in that they represent a failure in generation of the NK-cell lytic synapse at the end of the effector stages, as the abnormal lytic granules will not migrate along the microtubules to the MTOC.

Familial erythrophagocytic lymphohistiocytosis (FHL) types 3 and 4 are similar to CHS and HPS2, but are not associated with albinism demonstrating that the affected genes are not essential in melanocytes. FHL3 is caused by mutation in the UNC13D gene, which encodes MUNC13-4. FHL4 is caused by mutations in the STX11 gene, which encodes syntaxin-11.

Still further, of particular relevance for the methods of the invention are patients' populations diagnosed with one of autoimmune disorders, also referred to as disorders of immune tolerance, when the immune system fails to properly distinguish between self and non-self-antigens. It has been well established that T cells lymphocytes, and the NK cells in particular, play a pivotal role in the control of immune tolerance under normal conditions, and in T- and B-cell mediated human autoimmune disorders. The NK cells have been further implicated in rheumatoid arthritis, systemic lupus erythematosus, and in multiple sclerosis.

Thus, according to some embodiments, the methods of the invention, as well as any combinations, nanoparticles, cells, compositions and kits of the invention may be used for the treatment of a patient suffering from any autoimmune disorder. In some specific embodiments, the methods as well as any combinations, nanoparticles, cells, cell populations, compositions and kits of the invention may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, systemic lupus erythematosus (SLE), Eaton-Lambert syndrome, Goodpasture's syndrome, Greave's disease, Guillain-Barr syndrome, autoimmune hemolytic anemia (AIHA), Idiopathic thrombocytopenic purpura (ITP), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

Of particular interest to the present context is a condition denoted Graft versus Host Disease (GvHD) that may occur after an allogeneic transplant, wherein the donated transplant cells view the recipient's body as foreign. GvHD is a possible complication of high dose cancer treatment. It also happens after an allogeneic bone marrow or stem cell transplant that use very high doses of chemotherapy, sometimes with radiotherapy. The term ‘GvHD’ as meant herein encompasses all known form of GvHD, namely the acute GvHD (aGvHD), the chronic GvHD (cGvHD), and the late acute GVHD and overlap syndrome (with features of both aGvHD and cGvHD).

More specifically, the pathophysiology of aGvHD has been tightly linked to the activity and maturation of the donor T cells and NK cells that are transferred along with the marrow graft, i.e. cells that are directly responsible for recognition of antigenic differences on antigen-presenting cells of the host. Once activated, donor anti-host-specific T cells can mediate tissue destruction. GvHD continues to be a major life-threatening complication after allogeneic bone marrow transplantation.

In other words, use of the compounds as well as any combinations, nanoparticles, compositions and kits according to the present invention is particularly relevant in patients diagnosed with one of the types of GvHD. Such patients may be recognized by specific manifestation of symptoms. In the classical sense, a GvHD is characterized by selective damage to the liver, skin (rash), mucosa, and the gastrointestinal tract. Other types of GvHD may further involve the hematopoietic system, e.g., the bone marrow and thymus, and the lungs in the form of immune-mediated pneumonitis. Differential diagnosis of GvHD is further based on specific biomarkers.

In specific embodiments, the compounds as well as any combinations, nanoparticles, compositions and kits according to the present invention are applicable to patients that are at risk of developing GvHD. For example, recipients who have received peripheral blood stem cells/bone marrow from an HLA mismatched related donor (or from an HLA matched unrelated donor) have an increased risk of developing a GvHD.

In other words, it is meant that the compositions and methods of the present invention can be applied to prevent the development of aGvHD.

As indicated above, the in some further embodiments, the combinations of the invention or any nanoparticles, formulations, cells or compositions thereof, may be applicable in boosting the immune response of a subject suffering from a secondary immunosuppression caused by chemotherapy, specifically, treatment with a chemotherapeutic agent.

A “chemotherapeutic agent” or “chemotherapeutic drug” (also termed chemotherapy) as used herein refers to a drug treatment intended for eliminating or destructing (killing) cancer cells or cells of any other proliferative disorder. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Various different types of chemotherapeutic drugs are available. A chemotherapeutic drug may be used alone or in combination with another chemotherapeutic drug or with other forms of cancer therapy, such as a biological drug, radiation therapy or surgery.

Certain chemotherapy agents have also been used in the treatment of conditions other than cancer, including ankylosing spondylitis, multiple sclerosis, hemangiomas, Crohn's disease, psoriasis, psoriatic arthritis, rheumatoid arthritis, lupus and scleroderma.

Chemotherapeutic drugs affect cell division or DNA synthesis and function and can be generally classified into groups, based on their structure or biological function. The present invention generally pertains to chemotherapeutic agents that are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents.

However, several chemotherapeutic drugs may be classified as relating to more than a single group. It is noteworthy that some agents, including monoclonal antibodies and tyrosine kinase inhibitors, which are sometimes referred to as “chemotherapy”, do not directly interfere with DNA synthesis or cell division but rather function by targeting specific components that differ between cancer cells and normal cells and are generally referred to as “targeted therapies”, “biological therapy” or “immunotherapeutic agent” as detailed below.

More specifically, as their name implies, alkylating agents function by alkylating many nucleophilic functional groups under conditions present in cells. Examples of chemotherapeutic agents that are considered as alkylating agents are cisplatin and carboplatin, as well as oxaliplatin. Alkylating agents impair cell function by forming covalent bonds with amino, carboxyl, sulfhydryl, and phosphate groups in various biologically-significant molecules. Examples of agents which function by chemically modifying DNA are mechlorethamine, cyclophosphamide, chlorambucil and ifosfamide. An additional agent acting as a cell cycle non-specific alkylating antineoplastic agent is the alkyl sulfonate agent busulfan (also known as Busulfex).

In some particular embodiments, the immune-suppressive condition may be caused by treatment with oxaliplatin. More specifically, Oxaliplatin is a platinum-based chemotherapy drug in the same family as cisplatin and carboplatin. It is typically administered in combination with fluorouracil and leucovorin in a combination known as Folfox for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Oxaliplatin is marketed by Sanofi-Aventis under the trademark Eloxatin®.

Still Further, anti-metabolites (also termed purine and pyrimidine analogues) mimic the structure of purines or pyrimidines which are the building blocks of DNA and may thus be incorporated into DNA. The incorporation of anti-metabolites into DNA interferes with DNA syntheses, leading to abnormal cell development and division. Anti-metabolites also affect RNA synthesis. Examples of anti-metabolites include 5-fluorouracil (5-FU), azathioprine and mercaptopurine, fludarabine, cladribine (2-chlorodeoxyadenosine, 2-CdA), pentostatin (2′-deoxycoformycin, 2′-DCF), nelarabine, Floxuridine (FUDR), gemcitabine (Gemzar, a synthetic pyrimidine nucleoside) and Cytosine arabinoside (Cytarabine).

In yet some further embodiments, the compounds as well as any combinations, nanoparticles, compositions and kits of the invention may be applicable for boosting an immune-response in a subject treated with a chemotherapeutic agent that may be at least one Plant alkaloid and terpenoid. Plant alkaloids and terpenoids are agents derived from plants that block cell division by preventing microtubule function, thereby inhibiting the process of cell division (also known as “mitotic inhibitors” or “anti-mitotic agents”). Examples of alkaloids include the vinca alkaloids (e.g. vincristine, vinblastine, vinorelbine and vindesine) and terpenoids include, for example, taxanes (e.g. paclitaxel and docetaxel). Taxanes act by enhancing the stability of microtubules, preventing the separation of chromosomes during anaphase.

In further embodiments, the compounds, as well as any combinations, nanoparticles, compositions and kits used by the invention may be applicable for boosting an immune-response in a subject treated with chemotherapeutic agent that may be at least one Topoisomerase inhibitor. Topoisomerases are essential enzymes that maintain DNA topology (i.e. the overall three dimensional structure of DNA). Inhibition of type I or type II topoisomerases interferes with both transcription and replication of DNA by inhibiting proper DNA supercoiling. Type I topoisomerase inhibitors include camptothecins [e.g. irinotecan and topotecan (CPT11)] and examples of type II inhibitors include amsacrine, etoposide, etoposide phosphate, and teniposide. Still further, Anthracyclines (or anthracycline antibiotics) are a class of drugs used in cancer chemotherapy that are derived from the streptomyces bacterium. These compounds are used to treat many cancers, including leukemias, lymphomas, breast, uterine, ovarian, and lung cancers. These agents include, inter alia, the drugs daunorubicin (also known as Daunomycin), and doxorubicin and many other related agents (e.g., Valrubicin and Idarubicin). For example, the anthracycline agent Idarubicin acts by interfering with the enzyme topoisomerase II.

In further embodiments, the compounds, as well as any combinations, nanoparticles, cells, vectors, compositions and kits used by the invention may be applicable for boosting an immune-response in a subject treated with Doxorubicin. The chemotherapeutic agent Doxorubicin (also known by the trade name Adriamycin and by the name hydroxydaunorubicin) is an anthracycline antibiotic that is closely related to the natural product daunomycin, and like all anthracyclines, it works by intercalating DNA. The most serious adverse side effect of using this agent is the life-threatening heart damage. It is commonly used in the treatment of a wide range of cancers, including hematological malignancies, many types of carcinoma, and soft tissue sarcomas.

In certain embodiments, the compounds, as well as any combinations, nanoparticles, cells, vectors, vehicles, compositions and kits used by the invention may be applicable for boosting an immune-response in a subject treated with chemotherapeutic agent that may comprise at least one Cytotoxic antibiotics. The anthracyclines agents described above are also classified as “cytotoxic antibiotics”. Cytotoxic antibiotics also include the agent Actinomycin D (also known generically as Actinomycin or Dactinomycin), which is the most significant member of the actinomycines class of polypeptide antibiotics (that were also isolated from streptomyces). Actinomycin D is shown to have the ability to inhibit transcription by binding DNA at the transcription initiation complex and preventing elongation of RNA chain by RNA polymerase. Other cytotoxic antibiotics include bleomycin, epirubicin and mitomycin.

Still further, in some embodiments the combinations as well as any nanoparticles, cells, vectors, vehicles, compositions and kits of the invention or any nanoparticles or compositions thereof may be applicable for subjects suffering from immune-deficiency caused by immune-therapy or a biological therapy. More specifically, cancer vaccines, antibody treatments, and other “immunotherapies” are potentially more specific and effective and less toxic than the current approaches of cancer treatment and are generally termed “immunotherapy”, and therefore, an agent used for immunotherapy, is defined herein as an immuno-therapeutic agent. The term immunotherapy as herein defined (also termed biologic therapy or biotherapy) is a treatment that uses certain components of the immune system to fight diseases (e.g. cancer), by, inter alia, stimulating the immune system to become more efficient in attacking cancer cells (e.g., by administering vaccines) or by administering components of the immune system (e.g., by administering cytokines, antibodies, etc.).

In the last few decades immunotherapy has become an important part of treating several types of cancer with the main types of immunotherapy used being monoclonal antibodies (either naked or conjugated), cancer vaccines (i.e. that induce the immune system to mount an attack against cancer cells in the body) and non-specific immunotherapies.

Antibody-mediated therapy as referred to herein refers to the use of antibodies that are specific to a cancer cell or to any protein derived there-from for the treatment of cancer. As a non-limiting example, such antibodies may be monoclonal or polyclonal which may be naked or conjugated to another molecule. Antibodies used for the treatment of cancer may be conjugated to a cytotoxic moiety or radioactive isotope, to selectively eliminate cancer cells.

It should be noted that the term “biological treatment” or “biological agent”, as used herein refers to any biological material that affects different cellular pathways. Such agent may include antibodies, for example, antibodies directed to cell surface receptors participating in signaling, that may either activate or inhibit the target receptor. Such biological agent may also include any soluble receptor, cytokine, peptides or ligands. Non limiting examples for monoclonal antibodies that are used for the treatment of cancer include bevacizumab (also known as Avastin), rituximab (anti CD20 antibody), cetuximab (also known as Erbitux), anti-CTLA4 antibody and panitumumab (also known as Vectibix) and anti Gr1 antibodies. According to some embodiments, the anti-cancer agent is epidermal growth factor receptor (EGFR) inhibitor. According to some embodiments, the EGFR inhibitor is selected from the group consisting of: Cetuximab (Erbitux®), Panitumumab (Vectibix®), and necitumumab (Portrazza®). According to certain embodiments, the EGFR inhibitor is Cetuximab (Erbitux®).

More specifically, cancer vaccines as referred to herein are vaccines that induce the immune system to mount an attack against cancer cells in the body. A cancer treatment vaccine uses cancer cells, parts of cells, or pure antigens to increase the immune response against cancer cells that are already in the body. These cancer vaccines are often combined with other substances or adjuvants that enhance the immune response.

Non-specific immunotherapies as referred to herein do not target a certain cell or antigen, but rather stimulate the immune system in a general way, which may still result in an enhanced activity of the immune system against cancer cells. A non-limiting example of non-specific immunotherapies includes cytokines (e.g. interleukins, interferons). It should be thus appreciated that in some embodiments, the compounds, as well as any combinations, nanoparticles, compositions and kits used by the invention may be used as a combined supportive treatment for patients suffering from immune suppression. This supportive treatment may be combined with other supportive therapies as discussed herein.

Thus, in yet other embodiments, the combinations of the invention or any nanoparticles, cells, vectors, vehicles, kits, methods or compositions thereof may be applicable for subjects undergoing at least one of adoptive cell transfer, a cancer vaccine, antibody-based therapy, a hormone, a cytokine or any combination thereof.

As indicated above, in some embodiments, the compounds, as well as any combinations, nanoparticles, cells, vectors, vehicles, compositions and kits of the invention may be used for boosting the immune response in subjects undergoing radiotherapy. Radiation therapy or radiotherapy, often abbreviated RT, RTx, or XRT, is therapy using ionizing radiation, generally as part of cancer treatment to control or kill malignant cells and normally delivered by a linear accelerator. Radiation therapy may be curative in a number of types of cancer if they are localized to one area of the body. It may also be used as part of adjuvant therapy, to prevent tumor recurrence after surgery to remove a primary malignant tumor (for example, early stages of breast cancer). According to some specific embodiment, the radiation is ionizing radiation, which may be any one of X-rays, gamma rays and charged particles. In other embodiments, the radiation may be employed in the course of total body irradiation, brachytherapy, radioisotope therapy, external beam radiotherapy, stereotactic radio surgery (SRS), stereotactic body radiation therapy, particle or proton therapy, or body imaging using the ionizing radiation.

As indicated above, in some embodiments, the compounds, as well as any combinations, nanoparticles, cells, vectors, vehicles, compositions, methods and kits used by the invention may be used for boosting the immune response in subjects undergoing gene therapy. Gene therapy is the therapeutic delivery of nucleic acid polymers into a patient's cells as a drug to treat disease. The most common form uses DNA, optionally packed in a vector that encodes a functional, therapeutic gene to replace a mutated gene.

As indicated above, in some embodiments, the compounds, as well as any combinations, nanoparticles, cells, vectors, vehicles, compositions, methods and kits used by the invention may be used for boosting the immune response in subjects undergoing immunotherapy using checkpoint inhibitors. More specifically, immune checkpoint molecules are co-stimulatory and co-inhibitory molecules that act in coordination to modulate the immune response of autoreactive T cells Immune checkpoint molecules, like CTLA-4, TIM-3, PD-1, are negative regulators of immune responses to avoid immune injury. Recent studies have identified several new immune checkpoint targets, like lymphocyte activation gene-3 (LAG-3), T cell immunoglobulin and mucin-domain containing-3 (TIM-3), T cell immunoglobulin and ITIM domain (TIGIT), V-domain Ig suppressor of T cell activation (VISTA), and so on.

In some embodiments, a checkpoint inhibitor applicable in the method of the invention may be an antibody against an immune checkpoint molecule selected from the group consisting of human programmed cell death protein 1 (PD-1), PD-Ll and PD-L2, carcinoembryonic antigen-related cell adhesion molecule 1 (CEACAM1), lymphocyte activation gene 3 (LAG3), CD137, 0X40 (also referred to as CD134), killer cell immunoglobulin-like receptors (KIR), TIGIT, VISTA and any combination thereof. Each possibility represents a separate embodiment of the invention In some specific embodiments, immune check point inhibitors applicable in the present invention include but are not limited to Yervoy (ipilimumab) that targets CTLA-4, Keytruda (pembrolizumab) that targets PD-1 ligands, Opdivo (nivolumab) that targets PD-1, Tecentriq (atezolizumab) from Genentech, targets PD-L1, Bavencio (avelumab) blocks PD-L1, Imfinzi (durvalumab) targets PD-L1, Libtayo (cemiplimab-rwlc) that targets the PD-1 cellular pathway, and any combinations thereof.

It should be noted that the therapeutic methods disclosed by the invention may use any of the administration modes described herein before, in connection with the compositions of the invention, for example, administration by parenteral, intraperitoneal, transdermal, oral (including buccal or sublingual), rectal, topical (including buccal or sublingual), vaginal, intranasal and any other appropriate routes.

As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “effective amount” or “sufficient amount” used by the methods of the invention, mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

The terms “treat, treating, treatment” as used herein and in the claims mean ameliorating one or more clinical indicia of disease activity by administering a pharmaceutical composition of the invention in a patient having a pathologic disorder.

More specifically, the term “treatment”, as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

Still further, as mentioned above, the term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse re recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or even 100%.

With regards to the above, it is to be understood that, where provided, percentage values such as, for example, 10%, 50%, 100%, 120%, 500%, etc., are interchangeable with “fold change” values, i.e., 0.1, 0.5, 1.2, 5, etc., respectively.

The present invention relates to the treatment of subjects or patients, in need thereof. By “patient” or “subject in need” it is meant any organism who may be affected by the above-mentioned conditions, and to whom the monitoring and diagnosis methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the subject may be also any reptile or zoo animal More specifically, the methods of the invention are intended for mammals. By “mammalian subject” is meant any mammal for which the proposed therapy is desired, including human, and also non-human animals, specifically, non-human mammals such as livestock, equine, canine, and feline subjects, most specifically humans.

As indicated above, the methods of the invention provides in vivo activation of NK cells in inhibitory immunological synapses by administering to the treated subject a therapeutically effective amount of the combinations of the invention, specifically the combined inhibitors of at least one SHP proteins and at least one Cbl protein as disclosed by the invention, or any nano-particles, micro-particles, micellar formulations, vectors, vehicles and matrix thereof, or any combinations thereof with other therapeutic compounds. The invention therefore provides in vivo treatment of the subject that suffers from any of the described disorders. The combinations of the invention and specifically, the nano-particles provided by the invention target hematopoietic cell/s, specifically, NK cells in the treated subject and reduce the expression, stability and/or activity of at least one SHP protein and of at least one Cbl protein in the targeted NK cells, thereby leading to activation of these NK cells in inhibitory immunological synapses in the treated subject. This targeted activation enhances the immune response against any of the described disorders, specifically, cancer in the subject and therefore provide treatment thereof.

However, it should be understood that in some embodiments, the invention further encompasses and provides therapeutic methods involving ex vivo or in vitro steps for activating immune cells of the subject, or of a suitable donor, and introducing the activated cells, for example, NK cells, to the treated subject. In some specific embodiments, such methods may involve obtaining hematopoietic cells of a subject suffering from any of the described disorders or of a suitable donor (specifically, allogeneic donor), contacting in vitro/ex vivo the hematopoietic cell/s (e.g., T cell, B cells and/or NK cells) with an activating effective amount of the combinations of the invention or any nanoparticle, vehicles, vectors, micellar formulations, composition or kits thereof, and re-introducing the activated hematopoietic cells (that were activated in accordance with the methods of the invention) to the subject (adoptive transfer). Still further, it should be understood that hematopoietic cells of allogeneic or syngeneic subjects are also applicable in the methods of the invention.

More specifically, in some embodiments, the cells used by the methods of the invention, specifically, contacted with the combinations of the invention or with or any nanoparticle, vehicles, vectors, micellar formulations, composition or kits thereof provided by the invention may be cells, specifically, cells of an autologous source. The term “autologous” when relating to the source of cells, refers to cells derived or transferred from the same subject that is to be treated by the method of the invention. In yet some other embodiments, the cells used by the methods of the invention, specifically, the cells contacted with the combinations of the invention or with or any nanoparticle, vehicles, vectors, micellar formulations, composition or kits thereof provided by the invention may be cells of an allogeneic source, or even of a syngeneic source. The term “allogeneic” when relating to the source of cells, refers to cells derived or transferred from a different subject, referred to herein as a donor, of the same species. The term “syngeneic” when relating to the source of cells, refers to cells derived or transferred from a genetically identical, or sufficiently identical and immunologically compatible subject (e.g., an identical twin).

Still further, these cells, either of autologous, allogeneic or syngeneic source, may be than administered to the subject by adoptive transfer. The term “adoptive transfer” as herein defined applies to all the therapies that consist of the transfer of components of the immune system, specifically cells such as NK cells that are already capable of mounting a specific immune response. In such option, the activation of the NK cells by inhibiting the expression, stability and/or activity of at least one SHP protein and of at least one Cbl protein by the combinations of the invention is performed in cells of an autologous or allogeneic source, that are then administered to the subject, specifically, by adoptive transfer.

In yet some further embodiments, the invention further encompasses the in vivo or ex vivo therapeutic methods described herein performed in a subject suffering from any of the disorders disclosed herein before, that is further treated with an additional therapeutic agent, for example, an immune check point inhibitor. Thus, in some embodiments, the methods of the invention comprise the step of administering to a subject treated with at least one immune checkpoint inhibitor, an effective amount of the combinations of the invention or any nanoparticles, cells, vectors, vehicles, compositions and kits thereof. In some embodiments, the immune checkpoint inhibitor is any of the inhibitors specified herein before.

Still further aspect of the invention relates to at least one combination or any vehicle, matrix, nano- or micro-particle, micellar formulation or composition comprising said combination, for use in a method for treating, preventing, inhibiting, reducing, eliminating, protecting or delaying the onset of an immune-related disorder in a subject in need thereof. More specifically, the at least one combination comprising at least two compounds that specifically inhibit at least one of, the expression, activity and stability of at least one PTP, specifically, SHP protein and of at least one E3 ubiquitin-protein ligase, specifically, at least one member of the Cbl family.

It should be understood that in some embodiments, the at least one combination for use according to the invention refers to any of the combinations, cells, vectors, vehicles, compositions and kits thereof, as described by the invention herein above. In yet some further embodiments, any of the nanoparticle disclosed by the invention herein above are also applicable in this aspect as well. Thus, in some embodiments, the invention provides any of the combinations, nanoparticles and compositions disclosed by the invention for use in a method for treating, preventing, inhibiting, reducing, eliminating, protecting or delaying the onset of an immune-related disorder in a subject in need thereof. In yet some further embodiments, the invention provides any of the combinations, nanoparticles and compositions disclosed by the invention for use in a method for activating NK cells in an inhibitory immunological synapse (NKIS), specifically, in a subject in need thereof.

It should be noted that an immune-related disorder according to this aspect is any of the disorders specified herein before in connection with other aspects of the invention. Still further, in some embodiments, the at least one combination for use according to this aspect of the invention, may be used in a method of treating a subject that is already treated with at least one additional therapeutic agent, for example, an immune checkpoint inhibitor, or any of the other therapeutic agents disclosed by the invention in connection with other aspects.

As indicated above, the present invention further provides combined therapy involving the use of at least two compounds, specifically, the compounds that specifically inhibit at least one of, the expression, activity and stability of at least one SHP protein, compounds that specifically inhibit at least one of, the expression, activity and stability of at least one Cbl protein and optionally, at least one additional therapeutic agent, specifically, checkpoint inhibitor that may be administered either together in a pharmaceutical composition, or in separate compositions through different routes, dosages and combinations.

More specifically, the treatment of diseases and conditions with a combination of active ingredients may involve separate administration of each active ingredient. Therefore, a kit providing a convenient modular format of the different constituents of the compounds and related components required for treatment would allow the required flexibility in the above parameters.

Thus, in yet a further aspect, the invention relates to a kit comprising:

As a first component (a), at least one compound that specifically inhibit at least one of, the expression, activity and stability of at least one SHP protein, or any combinations of at least two of such compounds, or any vehicle, matrix, nano- or micro-particle, micellar formulation, cell or composition comprising said compound;

In a second component (b), at least one compounds that specifically inhibit at least one of, the expression, activity and stability of at least one member of the Cbl family, or any combinations of at least two of such compounds, or any vehicle, matrix, nano- or micro-particle, micellar formulation, cells or composition comprising said compound; and optionally (c), at least one additional therapeutic agent.

In some embodiments, any of the compounds described by the invention herein before, are also applicable for the kits of the invention. More specifically, the compound used by the kit of the invention may be at least one of a nucleic acid compound, an SMC, a peptide, aptamer and any combinations or mixtures thereof.

In yet some further embodiments, the kit of the invention may comprise (a) at least one siRNA molecule specifically directed against SHP-1; (b), at least one siRNA molecule specifically directed against Cbl-b; (c), at least one siRNA molecule specifically directed against c-Cbl; and optionally (d), at least one additional therapeutic agent.

It should be noted that the kits of the invention may comprise any of the components and compounds described above in connection with other aspects of the invention.

In yet some further embodiments, the invention provides the kits as described above for use in a method for treating, preventing, inhibiting, reducing, eliminating, protecting or delaying the onset of an immune-related disorder in a subject in need thereof. Still further, the invention provides the kits as described above for use in a method for activating NK cells in an inhibitory immunological synapse (NKIS), specifically, in a subject in need thereof.

In numerous embodiments, the compounds, as well as any combinations, nanoparticles, compositions and kits of the present invention may be administered in a form of combination therapy, i.e. in combination with one or more additional therapeutic agents. Combination therapy may include administration of a single pharmaceutical dosage formulation comprising at least one composition of the invention and additional therapeutics agent(s); as well as administration of at least one composition of the invention and one or more additional agent(s) in its own separate pharmaceutical dosage formulation. Further, where separate dosage formulations are used, compositions of the invention and one or more additional agents can be administered concurrently or at separately staggered times, i.e. sequentially. Still further, said concurrent or separate administrations may be carried out by the same or different administration routes.

In specific embodiments, compositions of the invention are administered with one or more therapeutic agents specifically relevant to infectious diseases, specifically, viral infection, cancer, a proliferative disorder, a graft versus host disease, an inflammatory disorder, an immune-cell mediated disorder and an autoimmune disorder.

In some embodiments of the present invention, it is contemplated that the other therapeutic agent may involve the administration or inclusion of at least one additional factor that may in some specific embodiments be any checkpoint inhibitor, for example, any antibody against an immune checkpoint molecule selected from the group consisting of human programmed cell death protein 1 (PD-1), PD-L1 and PD-L2, carcinoembryonic antigen-related cell adhesion molecule 1 (CEACAM1), lymphocyte activation gene 3 (LAG3), CD137, 0X40 (also referred to as CD134), killer cell immunoglobulin-like receptors (KIR), TIGIT, VISTA and any combination thereof. Each possibility represents a separate embodiment of the invention.

In some specific embodiments, immune check point inhibitors applicable in the present invention include but are not limited to Yervoy (ipilimumab) that targets CTLA-4, Keytruda (pembrolizumab) that targets PD-1 ligands, Opdivo (nivolumab) that targets PD-1, Tecentriq (atezolizumab) from Genentech, targets PD-L1, Bavencio (avelumab) blocks PD-L1, Imfinzi (durvalumab) targets PD-L1, Libtayo (cemiplimab-rwlc) that targets the PD-1 cellular pathway, and any combinations thereof.

More specifically in the present invention, it is contemplated that the other therapeutic agent may involve the administration or inclusion of at least one additional factor that may in some specific embodiments be selected from among EPO, G-CSF, M-GDF, EGF, SCF, GM-CSF, M-CSF, CSF-1, IL-1, IL-2, IL-3, IL-4, IL-5, IL-6, IL-7, IL-8, IL-9, IL-10, IL-11, IL-12, or other various interleukins, IGF-1, LIF, interferon (such as a, beta, gamma or consensus), neurotrophic factors (such as BDNF, NT-3, CTNF or noggin), other multi-potent growth factors (such as, to the extent these are demonstrated to be such multi-potent growth factors, flt-3/flk-2 ligand, stem cell proliferation factor, and totipotent stem cell factor), fibroblast growth factors (such as FGF), and analogs, fusion molecules, or other derivatives of the above.

Alternatively, treatment with the combinations of the invention or any nanoparticles or compositions thereof, may precede or follow the other agent treatment by intervals ranging from minutes to weeks. In embodiments where the other therapeutic agent and the compounds are administered separately, one would generally ensure that a significant period of time did not expire between the time of each delivery, such that the other agent and the compounds would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In some specific and non-limiting embodiments, the combinations of the invention or any nanoparticles or compositions thereof, may be applicable in combined treatment with G-CSF. Granulocyte-colony stimulating factor (G-CSF), also known as colony-stimulating factor 3 (CSF 3), is a glycoprotein that stimulates the bone marrow to produce granulocytes and stem cells and release them into the bloodstream. Functionally, it is a cytokine and hormone, a type of colony-stimulating factor, and is produced by a number of different tissues. The pharmaceutical analogs of naturally occurring G-CSF are called filgrastim and lenograstim. G-CSF also stimulates the survival, proliferation, differentiation, and function of neutrophil precursors and mature neutrophils.

In oncology and hematology, a recombinant form of G-CSF is used with certain cancer patients to accelerate recovery and reduce mortality from neutropenia after chemotherapy, allowing higher-intensity treatment regimens. G-CSF is also used to increase the number of hematopoietic stem cells in the blood of the donor before collection by leukapheresis for use in hematopoietic stem cell transplantation. G-CSF may also be given to the receiver in hematopoietic stem cell transplantation, to compensate for conditioning regimens.

All scientific and technical terms used herein have meanings commonly used in the art unless otherwise specified. The definitions provided herein are to facilitate understanding of certain terms used frequently herein and are not meant to limit the scope of the present disclosure. Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Experimental Procedures

Cells: Human YTS NK cell line, expressing inhibitory receptor KIR2DL1 (YTS-KIR2DL1); two types of the human lymphoma (B cells) 721.221 B-cell line, 721.221-HLA-Cw4, and 721.221-HLA-Cw7. NK92, a human NK lymphoma cell line, NK92 cells, expressing recombinant NKp46. Human erythroleukemia K562.

Primary peripheral blood mononuclear cell (PBMC) isolation: Human primary PBMCs were isolated from whole blood of healthy donors or patients, as previously described (Barda-Saad, M. et al. Nat. Immunol. 6, 80-89 (2005)). Briefly, human PBMCs were isolated from whole blood by Ficoll-Histopaque density gradient centrifugation (MP Biomedical). Isolated cells were cultured in RPMI 1640 (Sigma-Aldrich) supplemented with 10% FBS (Biological industries). All cells were grown at 37° C., under an atmosphere containing 5% CO₂.

Antibodies: Rabbit anti-SHP-1 (C-19), rabbit anti-GAPDH (FL-335) (Santa Cruz Biotechnology), mouse anti-Cbl-b (Santa Cruz Biotechnology), rabbit anti-Cbl (Santa Cruz Biotechnology), mouse anti-CD107a (Bio Legend).

Transfections by electroporation (AMAXA): Transfections of Cbl-b, c-Cbl, SHP-1 and non-specific siRNAs were conducted by using a Nucleofector® Kit & Device of the AMAXA Company. Transfections were conducted 48 hours before biochemical and effector experiments.

Lysis of NK cells: Cells were centrifuged and re-suspended in 20 μl lysis buffer. Cells were then placed on ice for 30 min and subsequently centrifuged at 14,000 rpm (or 20,000 g). The supernatant containing the cell lysate was collected and sample buffer (5×) was added to the cell lysates. Samples were heated at 100° C., for 5 min, followed by centrifugation.

Western blot (WB): Protein samples collected from cells were run on SDS-PAGE gel alongside a molecular marker (dual color, Bio-Rad) with known molecular masses to estimate the molecular masses of the protein samples. Samples were transferred to nitrocellulose membranes using a Trans blot semi-dry transfer cell (Bio-Rad). After the transfer, the membrane was shaken with a blocking buffer containing TBST (0.01% tween) with 3% dry milk (Sigma-Aldrich) or 5% BSA (Sigma-Aldrich), for 1 hour at room temp or overnight at 4° C. The membrane was then shaken with a primary antibody diluted in either dry milk or BSA and washed 3 times with TBST, for 1 hour at room temp or overnight at 4° C. Immunoreactive proteins were detected with either anti-mouse or anti-rabbit horseradish peroxidase (HRP)-coupled secondary antibody followed by detection with enhanced chemiluminescence (ECL) (PerkinElmer).

Membrane stripping: The nitrocellulose membrane underwent stripping with 10 ml of restore stripping buffer (Thermo).

Ca²⁺ release assay: YTS cells, 0.5×10⁶ to 1×10⁶ were incubated with 1 μg Fluo-3 per sample in RPMI 1640 medium at 37° C. for 45 min. The cells were washed, and resuspended in RPMI 1640 without phenol red containing 10 mM HEPES and 0.5 mM probenecid, and then were maintained at room temperature for 20 min. The cells were incubated at 37° C. for 5 min before measurements, first analyzed for 1 min to establish the basal intracellular Ca²⁺ concentration, and then mixed at a 1:1 ratio with target cells. Ca²⁺ influx was measured using the Synergy 4 spectrophotometer microplate reader (BioTek).

CD107a assay: YTS cells (3×10⁵) were co-incubated with 6×10⁵ 221 target cells at 37° C. for 2 hours in the presence of monensin (BioLegend). The cells were centrifuged, incubated with diluted anti-CD107a antibody for 30 min on ice, and washed twice. Staining with isotype-specific, Alexa flour-conjugated antibody was then performed on ice for 30 min. Cells were washed twice and analyzed by flow cytometry. As a negative control, cells that had not been pre-incubated with anti-CD107a antibody were incubated with goat anti mouse IgG1 Alexa Fluor-conjugated antibody.

Cytotoxicity assay: The cytolytic activity of NK cells against target cells was determined with a standard [³⁵S] Met release assay. Target cells were labeled with [³⁵S] Met (0.2 mCi/ml) for 12 to 16 hours and washed twice, and then 5000 cells were mixed with NK cells at an effector-to-target ratio of 10:1. Cells were then incubated for 5 hours at 37° C. in complete medium. The cells were then centrifuged at 200 g for 5 min, the supernatant was mixed with scintillation liquid, and analysis of the radioactive signal was performed with a b counter (Packard). Spontaneous release of [³⁵S] Met from an equal number of target cells was determined by adding 100 ml of complete medium to target cells that were incubated without NK cells. Maximal release was determined by adding 100 ml of 0.1 M NaOH to an equal number of target cells in the absence of NK cells. Finally, the percentage cell lysis caused by the NK cells was calculated by the following equation: % specific lysis=[(sample signal−spontaneous release)/(maximal release−spontaneous release)]×100.

Granzyme B release assay: A granzyme B release assay was conducted using Human Granzyme Elisa development Kit (Mabtech). Elisa reading was conducted at 450 nm wavelength at the synergy 4 spectrophotometer.

Flow cytometric analysis for mAb NKp46 staining: 3×10⁵ cells were incubated for 30 minutes on ice with 50 μl Supernatant cultivated from NKp46 hybridoma containing monoclonal antibodies for NKp46 activating receptor. The cells then were washed twice with PBS and incubated for 30 minutes on ice with a secondary antibody goat anti mouse IgG. The cells were washed twice and analyzed by flow cytometry.

Nanoparticles preparation: Four lipids were mixed: (i) Phosphatidylcholine (PC), (ii) Cholesterol (Chol), (iii) 1, 2-dihexadecanoyl-sn-glycero-3-phosphoethanolamine (DPPE) and (iv) DPPE marked with Rhodamine (Avanti). The lipid mix was frozen in liquid nitrogen and later heated at 37° C. Liposomes were coated with 6 mg of Hyaluronic acid(HA) (High molecular Weight-R&D) and 40 mg of 1-ethyl-3-(3-dimethylaminopropyl) carbodilimide (EDAC) (Sigma). For each 50 μg of liposomes, 400 mM of EDAC and 100 mM of N-Hydroxysuccinimide (NHS) were added (Sigma). The solution was incubated with 50 μg NKp46 antibodies at room temp overnight. Then the liposomes are lyophilized overnight to yield nanoparticles powder.

Cytokine storm: freshly-isolated PBMCs from healthy donors (1×10⁶ cells/ml) were incubated with empty NPs, SHP-1 and Cbls siRNA-loaded NPs, PBS (untreated), or PHA and LPS as positive controls. Following incubation, the supernatant from each sample was collected. The levels of human cytokines TNF-α, IL-6, IL-10 and IFN-γ were determined using human Mini ELISA Development kits (Peprotech).

Confocal microscopy: Cells were incubated with 50 μg rhodamine-labeled NPs for 24 hours. Cells were then centrifuged in a 24 well plate containing pol-L-lysine-treated cover slips at the bottom for 7 min at 1400 RPM. Following centrifugation, the cover slips containing the cells were washed, counter stained with 50 ng DAPI (Molecular probes) and images were acquired using the Leica TCS SP8 confocal microscope under a 63×objective lens.

Entrapment efficiency of siRNA: Non-specific siRNA (N.S siRNA) labeled with FITC was mixed with full-length recombinant protamine (Sigma-Aldrich) at a 1:5, siRNA:protamine molar ratio, in nuclease free water and incubated for 20 min at RT to form a complex. Immediately before use, the lyophilized NPs (300 μg total lipids) were rehydrated by adding 0.2 ml nuclease free water containing protamine-condensed siRNAs (1500 pmol siRNA). Free siRNA molecules were separated by ultra-centrifugation (Thermo Sorvall WX90), in order to measure the entrapment of N.S siRNA-FITC in the NPs the fluorescence intensity of FITC-siRNA (Ex/Em=485/535 nm) was measured using the Biotek Synergy 4 multi-mode microplate reader spectrophotometer (Bio-Rad Laboratories). siRNA concentration was measured before ultracentrifugation (total siRNA) and in the supernatant of the nanoparticles after ultracentrifugation (free siRNA). The entrapment efficiency of N.S siRNA-FITC in the NPs was calculated as follows: % siRNA entrapment=[(Total siRNA−Free siRNA)/(Total siRNA)]×100.

Mice: SCID/NOD mice were purchased from the Jackson Labs. All mice used were from colonies that were inbred and maintained under SPF conditions at the Bar-Ilan animal house. Housing and breeding of mice and experimental procedures were done according to the Bar-Ilan University Ethics Committee. Mice were subcutaneously injected with 3×10⁶ 721.221 Cw4-HLA in PBS mixed with Matrigel (CORNING) (1:1 v/v ratio). Mice weight, signs for stress or pain and tumor size was measured daily. Tumor size was measured using a digital caliper, and the following formula was used to calculate the tumor volume: Volume(mm³)=0.5×(major axis)×(minor axis)². Tumor average growth rate were calculated according to the formula: Average growth rate=(Day of last treatment−Day of first treatment)/(Last day tumor size−Initial tumor size).

In vivo efficacy studies: once tumors in xenograft mice reached the volume of 100 mm³, mice were randomly divided into two groups and were administered intratumorally (I.T), with irradiated YTS-KIR2DL1 and 300 μg NPs loaded with 1500 pmol siRNA (500 pmo1/each of SHP-1, Cbl-b and c-Cbl), every 72h for the total of 6 treatments. Tumor volumes were measured daily using a digital caliper. At the time of 24h post last-injection, mice were euthanized by CO2, and tumorous were harvested.

Statistic: Standard errors of the mean (SEM) and significances were calculated using Microsoft Excel. Statistical significances were calculated with Student's t-tests used for unpaired, two-tailed samples. In all cases, the threshold P-value required for significance was 0.05.

### Example 1

Specificity of SHP-1 and Cbls siRNA Gene Silencing in NK Cells

In order to suppress the key inhibitors of NK cell cytotoxicity, siRNA oligos were designed for SHP-1, Cbl-b and c-Cbl. More specifically, the following siRNAs were used: SHP-1 siRNA sense CCCUGACCCUGUGGAAGCAdTdT, as denoted by SEQ ID NO: 9, said siRNA targets the sequence as denoted by SEQ ID NO: 10, SHP-1 siRNA anti-sense UGCUUCCACAGGGUCAGGGdTdT, as denoted by SEQ ID NO: 11, said siRNA targets the sequence as denoted by SEQ ID NO: 12; Cbl-b siRNA sense CCCUUAUUUCAAGCCCUGAdTdT, as denoted by SEQ ID NO: 1, said siRNA targets the sequence as denoted by SEQ ID NO: 2, Cbl-b siRNA anti-sense UCAGGGCUUGAAAUAAGGGdTdT, as denoted by SEQ ID NO: 3, said siRNA targets the sequence as denoted by SEQ ID NO: 4; c-Cbl siRNA sense CUGUUGACAGACAGACUAAdTdT, as denoted by SEQ ID NO: 5, said siRNA targets the sequence as denoted by SEQ ID NO: 6, c-Cbl siRNA anti-sense UUAGUCUGUCUGUCAACAGdTdT, as denoted by SEQ ID NO: 7, said siRNA targets the sequence as denoted by SEQ ID NO: 8. Control non-specific sequences used are denoted by SEQ ID NO: 13 (sense) and SEQ ID NO: 14 (anti-sense).

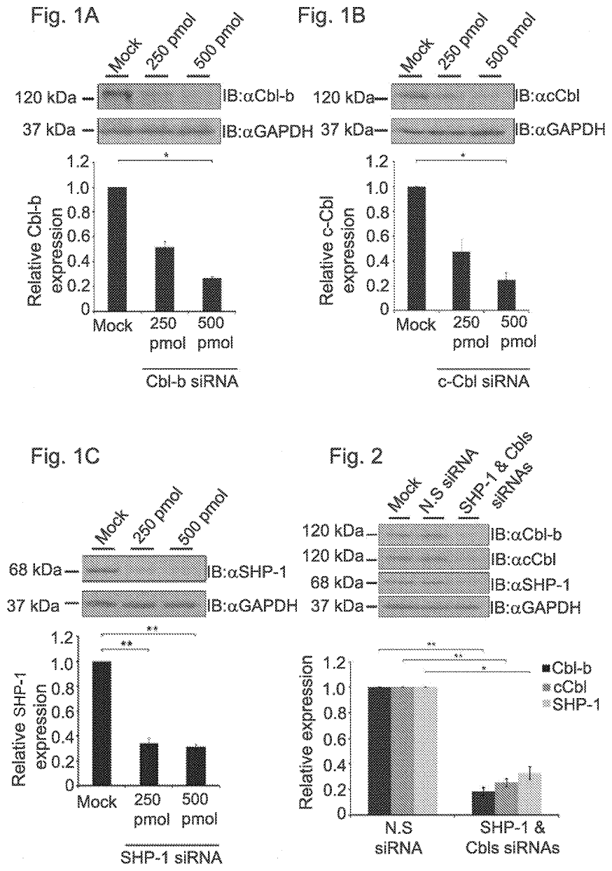

For this purpose, YTS KIR2DL1 cells were transfected with 250 or 500 pmol of Cbl-b (FIG. 1A), c-Cbl (FIG. 1B) or SHP-1 (FIG. 1C) siRNA and monitored for gene silencing efficiency after 48 hours. A significant decrease in all of the three proteins was detected relative to mock-transfected control cells (FIG. 1). Analysis by ImageJ densitometry revealed a decrease of 48±5% and 73±1% (Cbl-b), 53±10% and 75±6% (c-Cbl), and 65±3% and 69±2% (SHP-1) following siRNA gene silencing in concentrations of 250 pmol and 500 pmol, respectively (graphs below the blots). The specificity of SHP-1 and Cbls siRNA was confirmed by immuno-blotting (IB) with anti-GAPDH that served as a loading control. GAPDH expression was not influenced by the treatment with the specific siRNA (FIG. 1).

Next, the combined usage of SHP-1 and Cbls siRNA was tested for efficient gene silencing in YTS KIR2DL1 cells. As shown in FIG. 1, the most marked decrease in Cbl-b, c-Cbl and SHP-1 expression was obtained using 500 pmol siRNA. However, the objective was to condense those three siRNA together in lipid-based NPs with the optimal amount of siRNA that can be inserted into the NPs. Also, exposing cells to high amounts of siRNA can cause to off-target effects and might be harmful. Thus, it was decided to use 250 pmol of each siRNA.

For this purpose, YTS KIR2DL1 cells were transfected with 750 pmol of non-specific siRNA (N.S siRNA) or transfected with 250 pmol/each of Cbl-b, c-Cbl and SHP-1 siRNA. Gene silencing was determined by western blot analysis following 48 hours. A significant decrease of 82±3%, 75±3% and 67±5% was observed in the expression of Cbl-b, c-Cbl and SHP-1, respectively, relative to the N.S siRNA sample that served as a control (FIG. 2). The specificity of SHP-1 and Cbls siRNA was confirmed by TB with GAPDH that served as loading control. The expression of GAPDH was not affected by the addition of 750 pmol siRNA (FIG. 2). These data indicate that exposing YTS KIR2DL1 cells to total amount of 750 pmol of SHP-1 and Cbls siRNA together, results in efficient and specific gene silencing of those proteins.

### Example 2

### Ability of SHP-1 and Cbls Gene Silencing to Increase NK Cell Activation

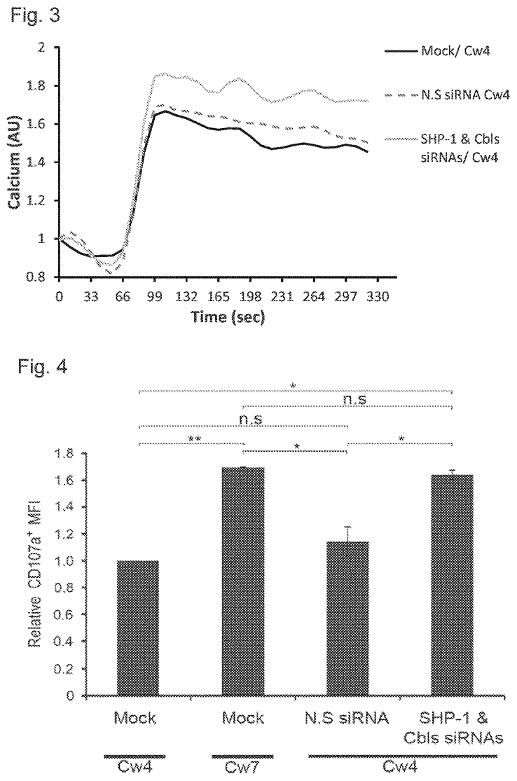

Next, functional assays were used to determine the efficiency of SHP-1 and Cbls gene silencing in enhancing NK cell activation. NK cell activation is mediated by the rapid increase in intracellular calcium flux that regulates multiple cellular functions including transcriptional activity and cytoskeleton rearrangements (Schwarz, E. C., et al. Biochim. Biophys. Acta—Mol. Cell Res. 1833, 1603-1611 (2013)). The ability of SHP-1 and Cbls siRNA to increase the intracellular calcium flux in YTS KIR2DL1 cells was investigated by incubating them with 721.221 HLA-Cw4 target cells. These malignant cells express the human leukocyte antigen (HLA) of allotype Cw4, the ligand for the KIR2DL1 inhibitory receptor. Thus, this incubation induces an inhibitory interaction which abrogate killing by NK cells. As can be seen in FIG. 3, intracellular calcium levels following inhibitory interactions with Cw4 target cells were markedly higher in YTS KIR2DL1 transfected with SHP-1 and Cbls siRNA relative to the levels observed in YTS KIR2DL1 transfected with N.S siRNA or the mock sample.

As part of the NK cell cytotoxic response, there is a release of cytotoxic granules containing proteins such as perforin and granzyme B, in a process known as degranulation. During the NK cell degranulation, lysosomal-associated membrane protein-1 (LAMP-1 or CD107a) and -2 (LAMP-2 or CD107b) transiently appear on the surface of NK cells (Paul, S. & Lal, G. Front. Immunol. 8, 1124 (2017)). To determine the ability of SHP-1 and Cbls siRNA to enhance NK cell degranulation, the expression of CD107 on NK cell was monitored as an indirect measurement of NK cell cytolytic function. For this purpose, YTS KIR2DL1 were incubated with 721.221 Cw4 target cells which induce inhibitory interactions or with 721.221 Cw7 target cells which induce activating interactions. 721.221 Cw7 cells express HLA of allotype Cw7, which is not recognized by the KIR2DL1 receptor, thus promoting NK cell activation. YTS KIR2DL1 transfected with SHP-1 and Cbls siRNA exhibited a significant increase of 1.64±0.04 in their ability to secrete cytolytic granules following inhibitory interactions compared to mock-transfected cells that were subjected to inhibitory interactions (FIG. 4). Interestingly, this activity resembled the degranulation level during an activating response (SHP-1 and Cbls siRNA/Cw4 1.64±0.04; Mock/Cw7 1.69±0.003, P=0.4).

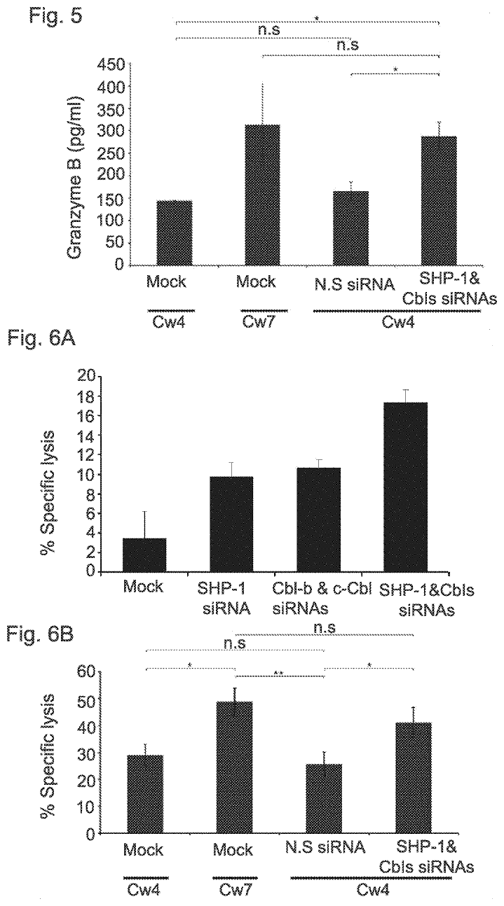

In addition, the release of cytotoxic molecules granzyme B was examined as an indirect measurement of NK cell cytolytic function. A main pathway used by NK cells to eliminate target cells is via exocytosis of granule components such as granzyme B, a serine protease, which activates caspase molecules leading to induction of apoptosis of target cells (Martinez-Lostao, L., et al. Clin. Cancer Res. 21, 5047 LP-5056 (2015)). To this end, YTS KIR2DL1 cells were incubated with Cw4 target cells for 2 hours and the release of granzyme B was assessed using ELISA assay. As expected, incubation of YTS KIR2DL1 with activating Cw7 target cells induced a release of 312.2±90.6 pg/ml granzyme B, relative to a significant smaller amount of 144.2±0.3 pg/ml that was released following incubation with inhibitory Cw4 target cells (FIG. 5). Interestingly, YTS KIR2DL1 cells pretreated with SHP-1 and Cbls siRNA that were incubated with inhibitory Cw4 cells exhibited a significant increase of granzyme B levels compared to YTS KIR2DL1 pretreated with N.S siRNA (287.9±26.1 pg/ml and 165.4±16.3 pg/ml, respectively, P<0.01) (FIG. 5). The granzyme B levels that were released following SHP-1 and Cbls gene silencing under inhibitory interactions were insignificantly different to those obtained under activating interactions (SHP-1 and Cbls siRNA/Cw4 287.9±26.1 pg/ml; Mock/Cw7 312.2±90.6 pg/ml, P=0.8). Taken together these results highlight the fact that gene silencing of SHP-1 and Cbls siRNA in inhibitory NK cells transform them into activating NK cells as detected by their cytolytic response to cancer cells.

The next step was to determine the efficacy of SHP-1 and Cbls siRNA to enhance NK cell-mediated killing using a direct approach. For this purpose, a standard radioactive [³⁵S] Met release assay was performed. FIG. 6A shows that the observed combined effect is synergistic. The inventors have further repeated the experiment. Briefly, inhibitory Cw4 or activating Cw7 target cells were incubated with [³⁵S] Met for 12 hours followed by a series of washes. The [³⁵S] Met-loaded target cells were then incubated for 5 hours with YTS KIR2DL1 cells that were either mock-transfected or transfected with N.S or SHP-1 and Cbls siRNA. The supernatant was collected and mixed with scintillation liquid. Analysis of the radioactive signal was performed using a β counter (Packard). As expected, the cytotoxicity observed in mock-transfected YTS KIR2DL1 cells that were incubated with activating Cw7 target cells was significantly higher relative to the cytotoxicity detected following inhibitory interactions (Mock/Cw7 48.8±5%; Mock/Cw4 29±3.8%, P=0.007). Inhibitory NK cells transfected with SHP-1 and Cbls siRNA exhibited a higher cytotoxicity response compared to YTS KIR2DL1 that were either mock-transfected or transfected with N.S siRNA (SHP-1 and Cbls siRNA/Cw4 41.1±5.6%, Mock/Cw4 29±3.8% or N.S siRNA/Cw4 25.7±4.4%). Importantly, NK cells transfected with SHP-1 and Cbls siRNA that were subjected to inhibitory conditions presented similar cytotoxicity response to the mock-treated NK cells under activating conditions (SHP-1 and Cbls siRNA/Cw4 41.1±5.6, Mock/Cw7 48.8±5%, P=0.3) (FIG. 6B).

### Example 3

### Synergistic Effect of Combined Silencing SHP-1 and Cbls in Enhancement of NK Cell Function

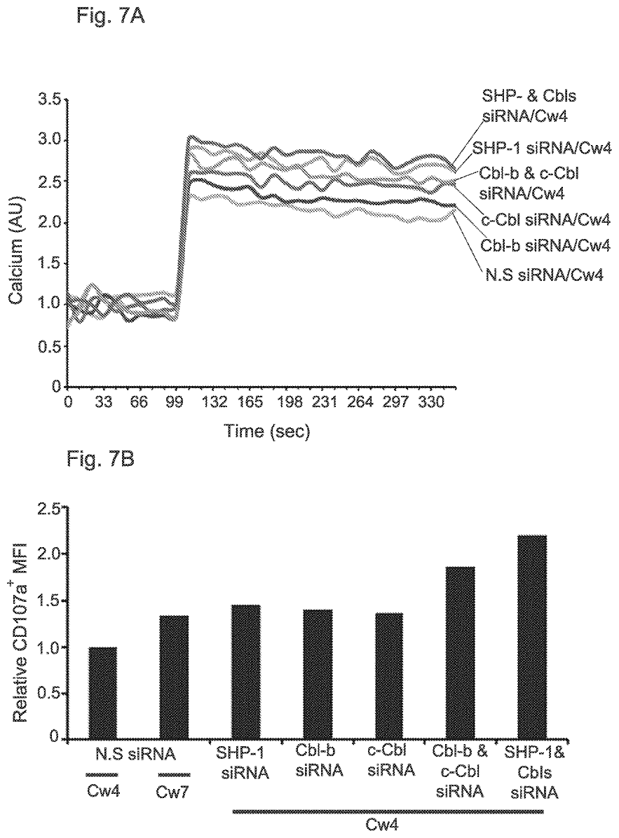

Encouraged by the synergistic effect of combined targeted elimination of all three molecules using the siRNAs of the invention, the inventors further used additional functional assays to determine the synergistic effect of SHP-1 and Cbls gene silencing in enhancing NK cell activation. In order to test the impact of SHP-1 and Cbl repression of NK cell activity, a common immune-editing pathway which inhibits NK cells against various cancers-high surface expression of matched HLA expression was next stimulated. To this end, YTS-2DL1 cells were incubated with 721.221 target cells that overexpress the inhibitory human leukocyte antigen (HLA)-Cw4, which induce NK cell inhibition (221-Cw4 cells), or with 721.221 targets expressing the irrelevant HLA-Cw7 (221-Cw7 cells) molecule, inducing NK cell activation. As can be seen in FIGS. 7A and 7B, intracellular calcium levels and NK cell degranulation were higher in YTS-2DL1 gene silenced for both SHP-1 and Cbls and interacted with inhibitory 221-Cw4 cells, relative to the levels observed in YTS-2DL1 transfected with N.S siRNA or siRNA targeting Cbl-b, c-Cbl and SHP-1 individually. Interestingly, YTS-2DL1 transfected with combination of SHP-1 and Cbls siRNAs and incubated with 221-Cw4 showed higher degranulation levels compared to YTS-2DL1 N.S siRNA transfected cells under activating interactions (with 221-Cw7 cells) (FIG. 7B). This data demonstrates that Cbl-b, c-Cbl, and SHP-1 regulate the NK cell activation threshold. These results therefore demonstrate the synergistic and unexpected effect of these inhibitory checkpoint molecules in increasing NK cell function.

### Example 4

### Recognition of NK Cells Using NKp46 Monoclonal Antibody

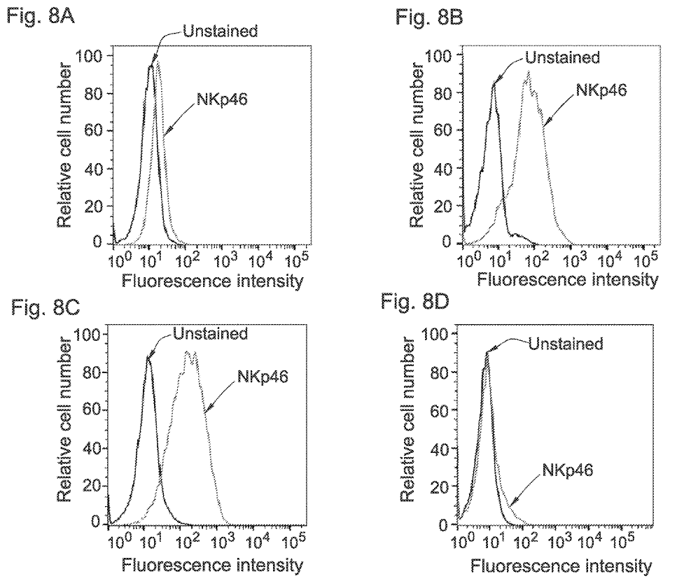

Having demonstrated the ability of SHP-1 and Cbls gene silencing to efficiently and specifically increase NK cell cytotoxicity to cancer cells, it was aimed to create lipid-based NPs that would serve as carriers for the siRNA. In order to achieve a specific recognition of NK cells by the siRNA-containing NPs, the NPs were coated with antibodies recognized NK cells and specifically the activating receptor NKp46, which is expressed exclusively on most NK cells and thus serves as a marker for their identification. Furthermore, the usage of this antibody does not affect NK cell function. Thus, NKp46 activating receptor will serve as a marker for targeting NK cells and will attribute to the specificity of the NPs to deliver siRNA for silencing the desired genes (Montaldo, E. et al. Cytom. Part A 83, 702-713 (2013)). To produce a specific NKp46 antibody, a hybridoma that continuously secretes the monoclonal NKp46 antibodies was used. The ability of cultivating NKp46 monoclonal antibodies was further investigated to specifically recognize their receptor on NK cells. To this end, the NK-92 cell line was used that naturally expresses minor endogenous amounts of NKp46 (NK92-NKp46) and an NK-92-cell line that over expresses NKp46 (NK92-NKp46+). In addition, the expression of NKp46 was examined in YTS KIR2DL1 cells that served as an in vitro model in the inventor's previous experiments. K562, chronic myeloid leukemia (CML) cell line that does not express NKp46 was used as a negative control. As can be seen in FIG. 8 a high staining of NKp46 was detected in NK92-NKp46⁺ cells (FIG. 8B) and only a weak staining was detected in the NK92-NKp46⁻ cells (FIG. 8A). In addition, YTS KIR2DL1 cell line also presented a high expression of NKp46 (FIG. 8C). NKp46 expression was not detected in K562 cells (FIG. 8D). These results demonstrate that the monoclonal anti-NKp46 antibody that was employed, recognizes its receptor on NK cells in a specific manner with a relative high affinity.

### Example 5

Preparation of NKp46 Targeted Stabilized Nanoparticle (NPs) for Efficient Delivery of SHP-1 and Cbls siRNA into NK Cells

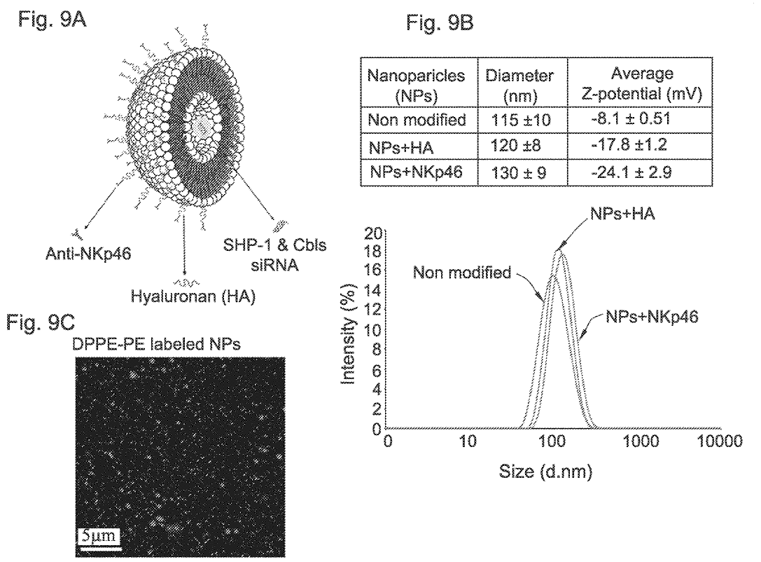

NPs that are cell-specific targeted are superior to the currently used therapeutic approaches using chemotherapy or biological agents including antibodies due to their potential to provide selective delivery of the therapeutic agents with significantly reduced side effects (Singh, R. & Lillard, J. W. Exp. Mol. Pathol. 86, 215-223 (2009)). Liposomal nanoparticles are pharmaceutically proven delivery vehicles that can be encapsulated a therapeutic agent and also display ligands that target cell-surface receptors (Torchilin, V. P. Nat. Rev. Drug Discov. 4, 145-160 (2005)). Leukocytes are difficult targets for delivery due to their resistance to conventional transfection reagents, and their dispersion within the body, making it difficult to successfully localize or passively deliver molecules via systemic administration (Bitko, V., et al. Nat. Med. 11, 50-55 (2005)). To circumvent these obstacles, NK cell-specific targeted NPs were utilized. Specifically, these NPs are composed of phosphatidylcholine (PC), dipalmitoylphosphatidylethanolamine (DPPE), and cholesterol (Chol) at molar ratios of 3:1:1 (PC:DPPE:Chol), that were prepared by a lipid-film method (Yerushalmi, N. & Margalit, R. BBA—Biomembr. 1189, 13-20 (1994)). The lipid film was hydrated with phosphate buffered saline (PBS) to create multilamellar liposomes. The resulting multilamellar liposomes were then extruded into unilamellar nano-scale liposomes (ULNL) with a hand-operated Mini-Extruder™ device (Avanti Polar Lipids, Inc.) at 65° C. (above the transition temperature). The extrusion was carried out in two steps using decreasing pore-sized polycarbonate membranes (200 and 100 nm) (Nucleopore, Whatman), with 10 cycles of extrusion per pore-size. ULNL were surface-modified with high molecular weight glycosaminoglycan hyaluronic acid (HA) (>950 kDa, R&D Systems), as previously described (Landesman-Milo, D. et al. Cancer Lett. 334, 221-227 (2013)). The HA stabilizes the NPs and protect them. It also serves as a scaffold to which antibodies can be attached. The HA-NPs were separated by ultra-centrifugation and washed 3 times. They were then coupled to NKp46 mAbs using an amine-coupling method (Peer, D., et al; Science (80). 319, 627-630 (2008)). The resulting NPs were divided into aliquots and were frozen for 2-4 hours at −80° C. followed by lyophilization for 24 hours. This led to the production of neutral unilamellar nano-scale modified liposomes. The use of neutral lipids to generate liposomes was proved to be safer than the usage of charged lipids or polymers (Kedmi, R., et al. Biomaterials 31, 6867-6875 (2010)). To summarize, a multilayer lipid-based nanoparticle coated with monoclonal anti-NKp46 antibody was prepared in large scale (FIG. 9A). To achieve the desired effect, SHP-1 and Cbls siRNA were condensed into these NPs by using protamine, which is a small positively charged protein that enhances the entrapment of nucleic acids by neutralizing their negative charges, enabling their condensation inside the NPs. To monitor the NP preparation process, the size and charge of the NPs were examined in the preparatory stages using Dynamic light scattering (DLS) (FIG. 9B). To enable detection of the NPs, DPPE labeled with Rhodamine red (DPPE-PE, excitation/emission: 560/583 nm) was incorporated into the lipid mixture at molar ratios of 6:2:1.95:0.05 (PC:Chol:DPPE:DPPE-PE), allowing their visualization using confocal microscopy as can be seen in FIG. 9C.

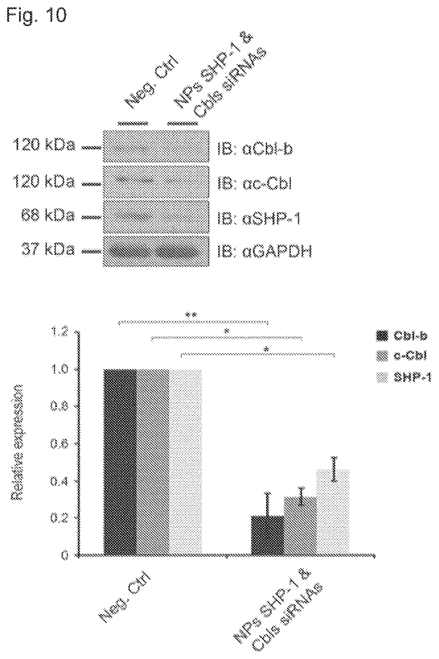

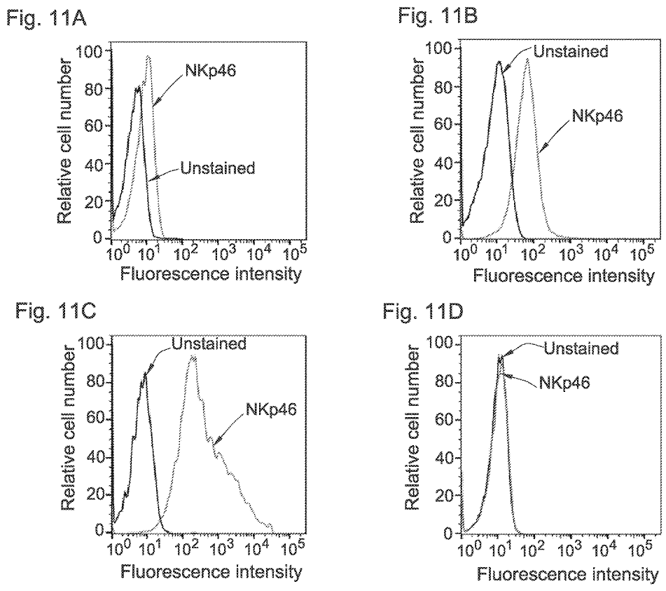

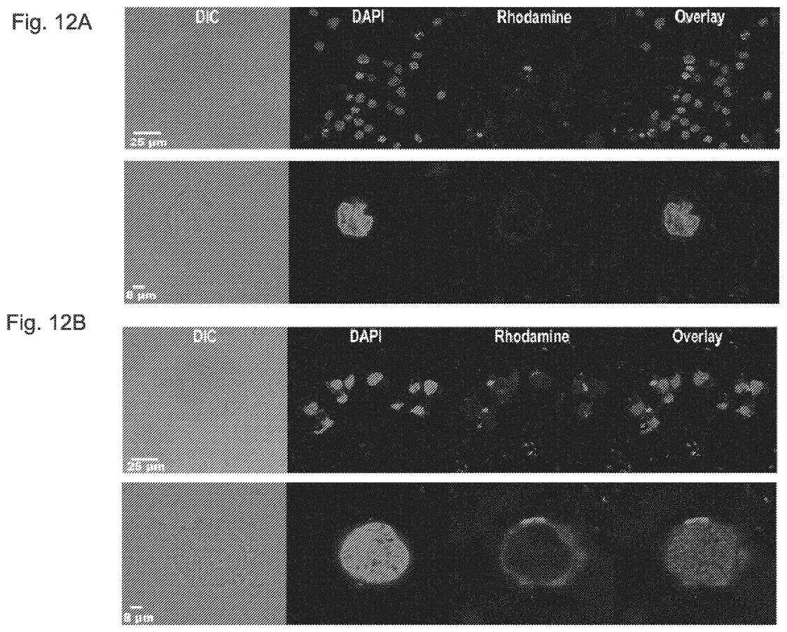

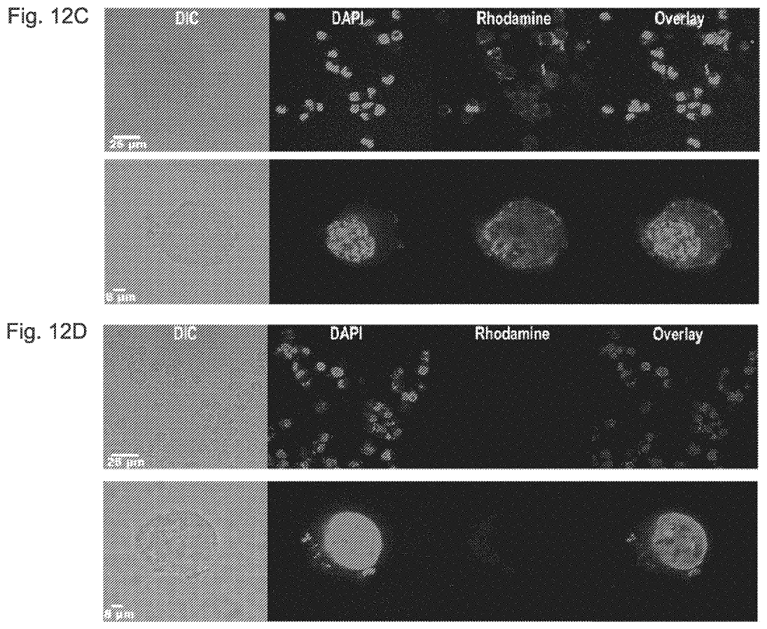

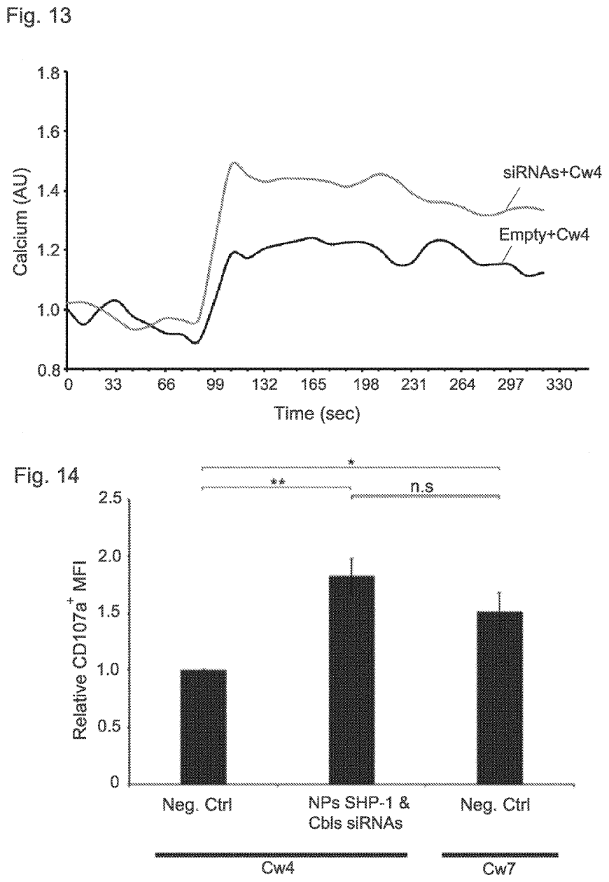

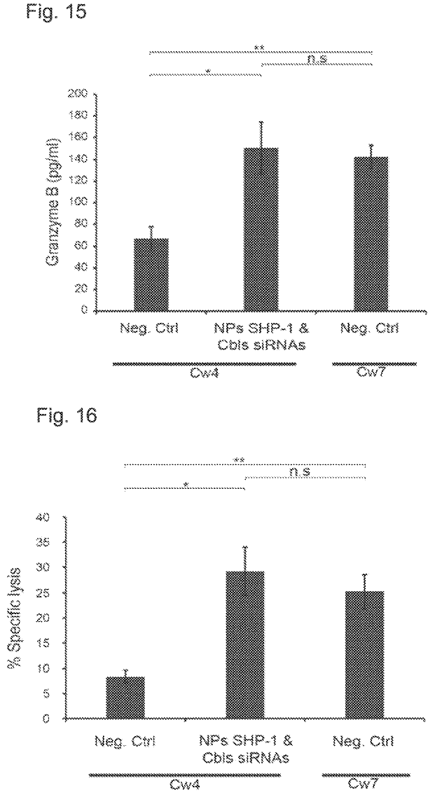

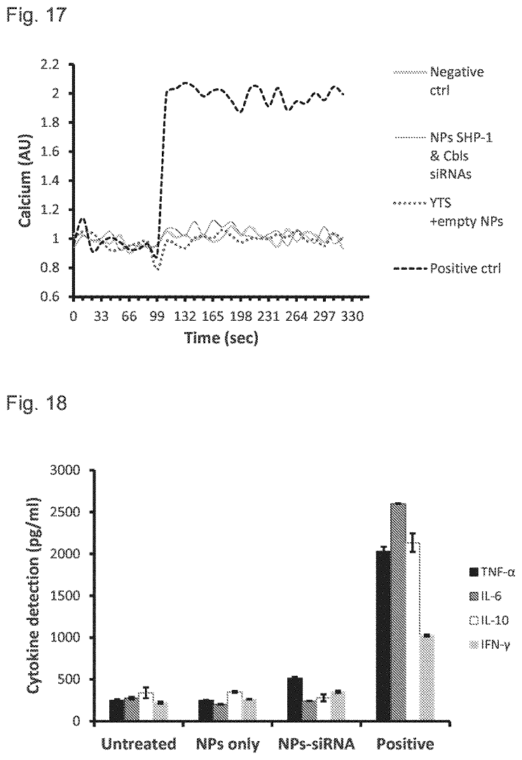

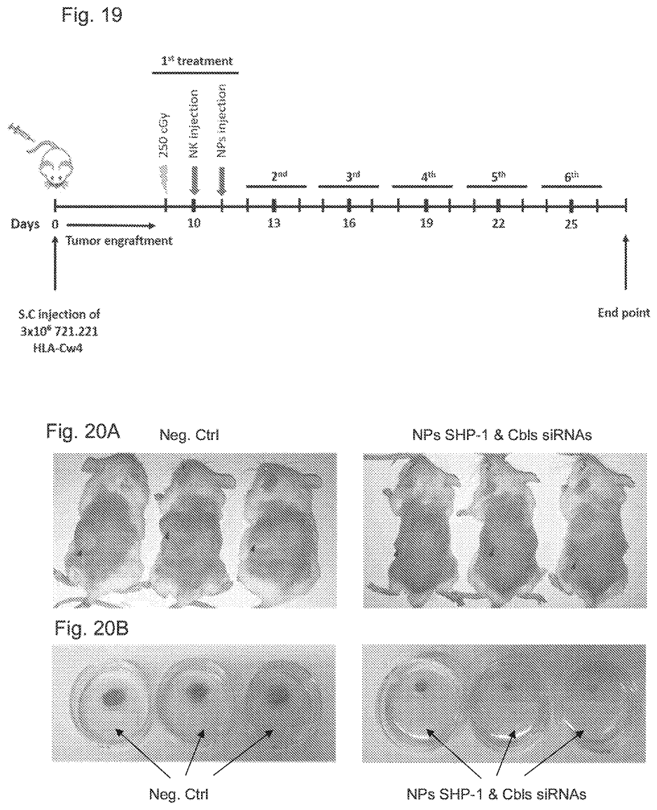

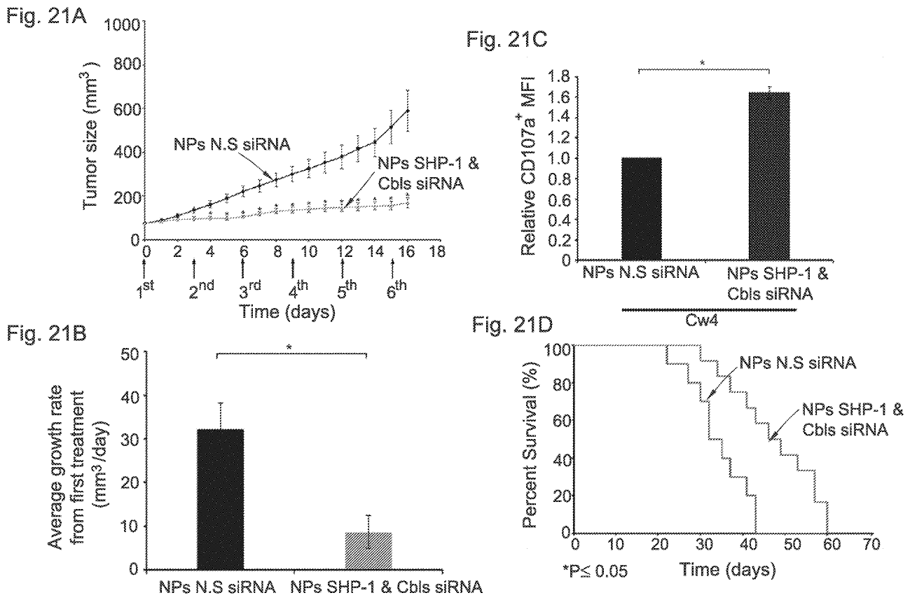

To validate that the NPs are able to efficiently deliver the siRNA, YTS KIR2DL cells were used which express the NKp46 receptor (FIG. 8C). To this end, 500 pmol/each of Cbl-b, c-Cbl and SHP-1 siRNA were condensed using protamine in a ratio of 1:5 (siRNA:protamine) into 300 μg NPs, followed by their incubation with YTS KIR2DL1 cells at a concentration of 5×10⁵ cells/ml. After 48 hours, the treated cells were lysed, and gene silencing was determined by western blot analysis. Densitometry analysis by ImageJ revealed a reduction of 80±4%, 69±6% and 54±12% in the expression of Cbl-b, c-Cbl and SHP-1, respectively, in the siRNA-loaded NPs, relative to empty NPs (Neg. ctrl) (FIG. 10). GAPDH served as a loading control; the GAPDH expression was not influenced by the treatment with the specific siRNA.

Having demonstrated an efficient delivery and loading of the siRNA into YTS KIR2DL1 cells, the specificity of NKp46 antibody-coated NPs was then validated to selectively target NK cells expressing NKp46. For this purpose, NK-92 cell line were used that expresses minor amounts of NKp46 (NK92-NKp46⁻) as well as NK-92-cell line that over expresses NKp46 (NK92-NKp46+). In addition, NP uptake was examined in YTS KIR2DL cell line. K562 cells were also used which are categorized and characterized as CML cell line that does not express NKp46. All the cells were incubated with 50 μg rhodamine-labeled NPs for 24 hours, after which the cells were collected, washed twice with PBS and analyzed by flow cytometry. Since the cells were washed, only the incorporated NPs could be detected in the cells. As seen in FIG. 11, NK92-NKp46+ cells showed a high staining of the NPs (FIG. 11B), while only a minor staining was observed in NK92-NKp46⁻ cells, expressing low endogenous level (FIG. 11A). As expected, YTS KIR2DL1 cell line showed a high staining of the NPs, which is in accordance with their NKp46 expression (FIG. 11C). K562 cells did not show any staining of the NPs, this cell line does not express any endogenous levels of NKp46 receptor (FIG. 11D). These results demonstrate that the NKp46 antibody-coated NPs are highly specific and undergo internalization only in NK cells that express the NKp46 receptor.

The selective uptake and internalization of the NKp46 antibody-coated NPs was also confirmed using confocal microscopy. Cells were incubated with 50 μg rhodamine-labeled NPs for 24 hours. Cells were then centrifuged in a 24 well plate containing pol-L-lysine-treated cover slips at the bottom for 7 min at 1400 RPM. Following centrifugation, the cover slips containing the cells were washed, counter stained with 50 ng DAPI (Molecular probes) and images were acquired using the Leica TCS SP8 confocal microscope under a 63×objective lens. As can be seen in FIG. 12, NK92-NKp46⁻ showed a very little internalization of the NPs (FIG. 12A). No NP internalization was detected in K562 cells (FIG. 12D). However, a robust presence of the NPs was observed in NK92 NKp46+ and YTS KIR2DL1 cells (FIG. 12B-12C).

After confirming the uptake and internalization of the coated NPs in a NKp46-specific manner, it was aimed to check the efficiency of siRNA entrapment. For this purpose, N.S siRNA labeled with FITC (N.S FITC-siRNA) was incorporated into the NKp46 target specific NPs. In order to enhance siRNA entrapment efficiency, the N.S FITC-siRNA was condensed with protamine sulfate. Protamine sulfate is a positively charged molecule that reduces the negative charge of the siRNA, resulting in better encapsulation within the NPs. The encapsulation efficiency was evaluated by measuring the concentration of non-encapsulated siRNA. The encapsulation efficiency of siRNA was calculated as follows:

$$\% siRNA entrapment = \frac{( Total siRNA - Free siRNA )}{( Total siRNA )} \times 100$$

% siRNA entrapment—Encapsulated N.S FITC-siRNA into the NPs.

Total siRNA—The total N.S. FITC-siRNA with protamine sulfate.

Free siRNA—Non-encapsulated N.S FITC-siRNA in the supernatant.

N.S FITC-siRNA was incubated with protamine sulfate in 1:5 ratio for 30 minutes, and the fluorescence intensity of the N.S FITC-siRNA (Excitation/Emission=485/535 nm) was measured using a BioTek Synergy 4 Multi-Mode Microplate Reader (Bio-Rad Laboratories, Calif., USA). The fluorescence intensity measured was 1818 O.D (Free siRNA). Next, N.S FITC-siRNA with protamine sulfate was incubated with the liposomal NPs for another 30 minutes and were centrifuged at 33,000 rpm for 1 hour. After centrifugation, the fluorescence intensity of the supernatant measured was 394 O.D (Total siRNA). According to the above formula, the siRNA entrapment was 78%.

### Example 6

### Ability of the NKp46-Coated NPs to Improve NK Cell-Mediated Killing of Cancer Cells In Vitro

As shown herein above in FIGS. 3 to 7, gene silencing of SHP-1 and Cbls improves inhibitory NK cell-mediated killing of target cells, 721.221 HLA-Cw4 cancerous cells. Moreover, this combination display a clear synergistic effect on NK cell activation. Additionally, as detected by the Western Blot presented by FIG. 10, Cbl-b, c-Cbl and SHP-1 were knocked down using NKp46 antibody-coated NPs loaded with SHP-1 and Cbls siRNA. Next, the effect of these NKp46 antibody-coated NPs was examined on NK cell-mediated killing of the target 721.221 HLA-Cw4 cells. First, the ability of SHP-1 and Cbls gene silencing encapsulated by NPs to increase NK cell activation was determined. This was measured by intracellular calcium flux. NK cell activation is dependent on the rapid increase of intracellular calcium flux. Thus, the ability of NKp46 antibody-coated NPs loaded with SHP-1 and Cbls siRNA to increase the intracellular calcium flux in YTS KIR2DL1 cells by incubating them with 721.221 HLA-Cw4 target cells was analyzed. As presented in FIG. 13, intracellular calcium levels following inhibitory interaction with Cw4 target cells were markedly higher in YTS KIR2DL1 pretreated with SHP-1 and Cbls siRNA-loaded NPs (dark gray curve) relative to the levels observed in YTS KIR2DL1 that were served as negative control (gray curve).

Next, the ability of SHP-1 and Cbls gene silencing using NPs to enhance NK cell degranulation and cytotoxicity was determined. As part of the NK cell cytotoxic response, there is a release of cytotoxic granules containing proteins such as perforin and granzyme B in a process known as degranulation. During the NK cell degranulation, CD107 transiently appear on the surface of NK cells. To determine the ability of NPs loaded with SHP-1 and Cbls siRNA to enhance NK cell degranulation, the expression of CD107 was monitored on NK cell to measure NK cell cytolytic function. For this purpose, YTS KIR2DL1 were incubated with 721.221 Cw4 target cells which induce inhibitory interactions and with 721.221 Cw7 target cells which induce activating interactions. YTS KIR2DL1 pretreated with SHP-1 and Cbls siRNA-loaded NPs exhibited a significant increase of 1.82±0.31 in their ability to secrete cytolytic granules following inhibitory interactions compared to negative control cells that were subjected to inhibitory interactions (FIG. 14). Interestingly, this activity resembled the degranulation level during an activating response (SHP-1 and Cbls siRNA Cw4, 1.82±0.31; negative control/Cw7: 1.51±0.33, P=0.2).

In addition, the release of cytotoxic molecule granzyme B was examined as an indirect measurement of NK cell cytolytic function. To this end, YTS KIR2DL1 cells were incubated with Cw4 or Cw7 target cells for 2 hours and the release of granzyme B was assessed using ELISA assay. As expected, incubation of YTS KIR2DL1 with activating Cw7 target cells induced a release of 142.2±10.9 pg/ml granzyme B, relative to a significant smaller amount of 67.4±10.2 pg/ml that was released following incubation with inhibitory Cw4 target cells (FIG. 15). Interestingly, YTS KIR2DL1 cells pretreated with SHP-1 and Cbls siRNA-loaded NPs that were incubated with inhibitory Cw4 cells exhibited a significant increase of granzyme B levels compared to negative control. (150.3±24 pg/ml and 67.4±10.2 pg/ml, respectively, P=0.05) (FIG. 15). The granzyme B levels that were released following SHP-1 and Cbls gene silencing under inhibitory interactions were similar to those obtained under activating interactions (SHP-1 and Cbls siRNA loaded NPs Cw4 150.3±24 pg/ml; negative control/Cw7 142.2±10.9 pg/ml, P=0.7). Taken together these results highlight the ability of SHP-1 and Cbls siRNA-loaded NPs to enhance NK cell cytolytic response against target cells under inhibitory conditions.

As a conclusive evidence to the efficacy of SHP-1 and Cbls siRNA-loaded NPs to enhance NK cell-mediated killing, a direct approach was used. For this purpose, a standard radioactive [³⁵S] Met release assay was performed. Inhibitory Cw4 or activating Cw7 target cells were incubated with [³⁵S] Met for 12 hours followed by a series of washes. The [³⁵S] Met-loaded target cells were then incubated for 5 hours with YTS KIR2DL1 cells that served as negative control or incubated with SHP-1 and Cbls siRNA-loaded NPs. The supernatant was collected and mixed with scintillation liquid. Analysis of the radioactive signal was performed using a β counter (Packard). As expected, the cytotoxicity observed in the YTS KIR2DL1 cells that serves as negative control and were incubated with activating Cw7 target cells was significantly higher relative to the cytotoxicity detected following inhibitory interactions (negative control/Cw7: 25.2±3.4%; negative control/Cw4: 8.3±1.2%, P=0.005). Under inhibitory interactions YTS KIR2DL1 pretreated with SHP-1 and Cbls siRNA-loaded NPs exhibited a higher cytotoxicity response compared to YTS KIR2DL1 that were served as negative control (SHP-1 and Cbls siRNA-loaded NPs/Cw4 29.2±4.8%; negative control/Cw4 8.3±1.2%, P=0.01).

Importantly, NK cells pretreated with SHP-1 and Cbls siRNA-loaded NPs that were subjected to inhibitory conditions had similar cytotoxicity response to negative control NK cells under activating conditions (SHP-1 and Cbls siRNA loaded NPs/Cw4 29.2±4.8; negative control/Cw7 25.2±3.4%, P=0.5) (FIG. 16).

The ability of NKp46 antibody-coated NPs to increase NK cell activation was examined by measuring intracellular calcium flux. NK cell activation is dependent on the rapid increase in intracellular calcium flux. The ability of NKp46 antibody-coated NPs to increase the intracellular calcium flux in YTS KIR2DL1 cells was investigated without incubating them with target cells. As can be seen in FIG. 17, intracellular calcium levels following incubation with Cw7 activating target cells as positive control were remarkably higher in YTS KIR2DL1 cells (black curve) relative to the levels observed in YTS KIR2DL1 that were treated with PBS as negative control (gray curve), or in YTS KIR2DL1 cells that were pretreated with empty NPs (light gray curve) or with siRNA-loaded NPs (dark gray curve). These data indicate that NKp46 antibody-coated NPs (either empty or loaded with siRNA) do not induce intracellular calcium flux release by them self, and thus do not provoke NK cell activation.

One of the major difficulties in translating novel delivery strategy for RNAi into new therapeutic approach is undesirable immuno-stimulation that may induce a pro-inflammatory response leading to a cytokine storm. This stimulation could be attributed to the RNAi payload, the nano-vehicle or the combination of the nano-vehicle that is entrapping the RNAi payload. In order to evaluate the safety profile of NKp46 antibody-coated NPs, an in vitro cytokine induction study was performed using primary human lymphocytes, which examined the secretion of the inflammatory cytokines. Briefly, freshly-isolated PBMCs from healthy donors (1×10⁶ cells/ml) were incubated with empty NPs, SHP-1 and Cbls siRNA-loaded NPs, PBS (untreated), or PHA and LPS as positive controls. Following incubation, the supernatant from each sample was collected. The levels of human cytokines TNF-α, IL-6, IL-10 and IFN-γ were determined using human Mini ELISA Development kits (Peprotech). As shown in FIG. 18, almost no measurable amounts of the cytokines were detected in all samples except of the positive control samples. Furthermore, the cytokine concentrations were not significantly different from the control PBS samples. Thus, the safety of the NKp46 antibody-coated NPs was proved.

### Example 7

An In-Vivo System to Test the Effective Delivery of SHP-1 and Cbls siRNA and Determine the Efficacy of NKp46-NPs to Upregulate NK Cell Killing Activity

The ability of NKp46 antibody coated NPs to induce NK cell cytotoxicity towards tumor cells in-vivo was validated. Non-obese diabetic/severe combined immunodeficiency (NOD/SCID) mice were inoculated with 3×10⁶ 721.221 Cw4-HLA cells. When the tumors reached approximately 70 mm³ (Day 0), tumor-bearing mice were divided randomly into two groups. Mice were treated with irradiated YTS-KIR2DL1 and NPs containing SHP-1 and Cbls siRNA every 3 days, for the total of 6 injections. 5×10⁶ YTS-KIR2DL1 cells were irradiated at 250 cGy and then given 24 hours for recovering. Next, mice were treated with irradiated YTS KIR2DL1 following intratumorally (I.T) injection. After another 24 hours, mice were treated with NPs containing SHP-1 and Cbls siRNA following I.T injection (FIG. 19).

Tumor volume, mice weight and any symptom of morbidity were measured daily. After 24 hours from the last treatment, mice were sacrificed, and the tumors were harvested (FIGS. 20A, and 20B). Mice that were treated with SHP-1 and Cbls siRNA-loaded NPs had a significant attenuation in tumor size in comparison to the mice that served as negative control (FIG. 21A), 169.2±23.4 and 662.2±99.5 respectively (P<0.0001). SHP-1 and Cbls siRNA treated mice also showed significantly reduced average tumor growth rate of 8.7±3.8 compared to 36.8±6.15 of the negative control group (P<0.001) as measured from the first treatment and calculated as described in the experimental procedures (FIG. 21B). In order to further characterize and elucidate the role of NK cells in tumor clearance, tumors were extracted 24h post final treatment and homogenized. Cells were then ex vivo analyzed using flow cytometry, for the degranulation marker LAMP1 (CD107a) and the presence of Rhodamine red labeled NPs. Strikingly, NK cells positive for SHP-1 and Cbls siRNA loaded NPs showed significantly higher degranulation compared to the N.S siRNA treated cells (FIG. 21C).

Finally, the effects of NPs on the survival of 221-Cw4 NHL engrafted mouse models was studied. As demonstrated by the Kaplan-Meir curve (FIG. 21D), compared to mice treated with NK-specific NPs encapsulating NS siRNA, NK cells gene-silenced for SHP-1 and Cbls significantly extended the survival of the NHL engrafted mouse models.

This data clearly demonstrate that lipid-based NPs coated with NK cell targeted NKp46 are specific to their target, do not induce abnormal NK cell activation under steady state conditions, and greatly increase cytotoxic potential of NK cell both in-vitro and in-vivo, thereby extending the survival of the treated subjects.

### Example 8

### Personalized Natural Killer Immunotherapeutic Approach

Inhibitory checkpoint inhibition (ICI) therapy is not effective on all patients, and a considerable amount of patients develop over time resistance and severe side effects. Much of this resistance can be explained by intrinsic genetic and mutational variations of tumors between patients (Sharma, P., et al. Cell 168, 707-723 (2017)). Individual patient biomarkers are necessary to improve efficacy of treatment, and to decrease side effects induced by the treatment.

In context of NK cells this is apparent due to the variations in NK cell receptors repertoires. For example, splice variants of activating NK cell receptors such as NKp44 and NKp30 can actually induce NK cell inhibition (Shemesh, A. et al. Cancer Immunol. Immunother. 1-13 (2017). doi:10.1007/s00262-017-2104). Critically, it is also evident that inhibitory receptor repertoires exhibit high variations amongst cohorts of individuals, and difference in receptor diversity has large consequences on susceptibility for diseases (Sternberg-Simon, M. et al. Front. Immunol. 4, 65 (2013)). Additionally, differences in the individual's commensal bacteria, i.e. the microbiome, and bacteria in the tumor microenvironment itself, i.e. oncogenic bacteria, as well as viral pathogens such as Ebola virus, and even parasitic pathogens such as Leishmania donovanni and Toxoplasma gondii, by steric shielding of ligands for activating receptors or presenting ligands for inhibitory receptors, are becoming recognized as repressors of immune cell function. A non-limiting example for such bacteria is the Fusobacterium Nucleatum (FN) a bacterial strain linked to acceleration of colon cancer and immune cell suppression, and who's presence was shown to be significantly correlated with poor outcomes of cancer patients (Abed, J. et al. Cell. Infect. Microbiol. 7, 295 (2017)). The involvement of oncogenic bacterial variance between individuals provides an additional layer to the strategy of creating suitable personalized immunotherapeutic approaches. Given all of the evidence in variation between individuals' immune system and tumor resident bacterial profiles, treatment must be personally constructed to suit the specific profile of a given patient. A novel personalized immunotherapeutic approach is developed to improve NK cell activation in-vivo by using a nanoparticle tailored for a patient's tumor by increasing the cytotoxic ability of the patient NK cells. More specifically, infiltrating NK cells from tumors of patients are isolated and profiled for NK receptors that recognize and bind the tumor cells. Examples for some NK cell activating receptor include, but are not limited to NKp46, NKp44, NKp30, NKp80, CD27, LFA-1, CD16, NKG2D, CRTAM, DNAM-1 and any derivatives and splice variants thereof. Examples for NK cell inhibitory receptor include but are not limited to at least one of KIR, PD-1, CTLA4, Ly49, NKRP1A, CD94, NKGA2, TIGIT, CD96, TIM-3, LAG-3, CEACAM1, CTLA-4, LAIR-1, LILRB1, and any derivatives, splice variants, homologs and orthologs thereof or any combinations thereof. Specific NPs that are conjugated to antibodies and/or aptamers directed against at least one, at least two of the NK receptors, at least three, at least four, at least five, at least six, at least seven, at least eight, at least nine, at least ten or more, 11, 12, 13, 14, 15, 16, 17, 18, 19, 20, 21, 22, or more of the NK cells receptors indicated above, are prepared in accordance with the profiled expression in each individual. The key inhibitory signaling proteins that suppress NK cell cytotoxicity are targeted using cell-specific lipid NPs coated with specific antibodies or aptamers against the individually mapped relevant inhibitory NK cell receptors. For example, specific aptamers are found by performing screening of aptamers library against patients' whole cell samples (reviewed in Catuogno S., et al (2017) Biomedicines 5(3): 49). Additional optional antibodies for novel markers will be determined via single cell RNA seq of patient tumor biopsies samples or fluorescence staining of tumor sections for known markers. Lipid NPs are constructed that are relevant for a specific patient's tumor based on the expression of inhibitory receptors on the given patient's NK cells, the expression of inhibitory ligands on the patient's tumor, and on the presence or absence bacterial, viral or parasitic pathogens in the tumors. The siRNAs specific for SHP-1, Cbl-b, and c-Cbl, are encapsulated in NPs coated with antibodies/aptamers against the relevant inhibitory checkpoint molecules.

1-52. (canceled)

53. A combination comprising at least two compounds that specifically inhibit at least one of, the expression, activity and stability of at least one protein tyrosine phosphatase (PTP) and of at least one E3 ubiquitin-protein ligase, or any nano- or micro-particle, micellar formulation, vehicle, matrix, a composition or a kit comprising said combination.

54. The combination according to claim 53, wherein said at least one PTP is at least one Src homology region 2 (SH2) domain-containing phosphatase (SHP) protein, and wherein said at least one E3 ubiquitin-protein ligase is at least one member of the Casitas B-lineage Lymphoma (Cbl) E3 ubiquitin-protein ligase family.

55. The combination according to claim 54, wherein said at least two compounds comprise at least one nucleic acid molecule, each nucleic acid molecule is specific for at least one of: (i) at least one of SHP-1 and SHP-2; and (ii) at least one of Cbl-b, c-Cbl and Cbl-3, said nucleic acid molecule is a ribonucleic acid (RNA) molecule or any nucleic acid sequence encoding said RNA molecule, said RNA molecule is at least one of a double-stranded RNA (dsRNA), an antisense RNA, a single-stranded RNA (ssRNA), (guide RNA gRNA) and a Ribozyme, optionally, said dsRNA is at least one of small interfering RNA (siRNA), MicroRNA (miRNA), short hairpin RNA (shRNA) and PIWI interacting RNAs (piRNAs).

56. The combination according to claim 53, wherein said combination comprises at least two of: at least one siRNA molecule specific for SHP-1, at least one siRNA molecule specific for Cbl-b, and at least one siRNA molecule specific for c-Cbl, optionally, said siRNA molecule specific for SHP-1 comprises the nucleic acid sequence as denoted by at least one of SEQ ID NO: 9 and SEQ ID NO: 11, said siRNA molecule specific for Cbl-b comprises the nucleic acid sequence as denoted by at least one of SEQ ID NO: 1 and SEQ ID NO: 3, and said siRNA molecule specific for c-Cbl comprises the nucleic acid sequence as denoted by at least one of SEQ ID NO: 5 and SEQ ID NO: 7, or of any variants, homologs or derivatives thereof.

57. At least one nano- or micro-particle, micellar formulation, vehicle or matrix comprising at least one combination as defined in claim 53, wherein at least one of:
(a) said at least one combination is encapsulated within the intra-nanoparticle core or cavity of said nano- or micro-particle, micellar formulation, vehicle or matrix;
(b) at least one targeting moiety is associated directly or indirectly with the outer nanoparticle surface of said nano- or micro-particle, micellar formulation, vehicle or matrix; and
(c) said at least one targeting moiety is at least one of an antibody, an aptamer, a ligand or any combinations thereof, that specifically recognizes and binds at least one molecule expressed on the surface of at least one hematopoietic cell.

58. The at least one nano- or micro-particle, micellar formulation, vehicle or matrix according to claim 57, wherein said hematopoietic cell is a natural killer (NK) cell, optionally, wherein at least one of:
(a) said nano- or micro-particle, micellar formulation, vehicle or matrix is connected directly or indirectly by the outer nanoparticle surface thereof with at least one antibody, at least one aptamer or any combinations thereof, that specifically recognize and bind at least one of at least one NK cell activating receptor and at least one NK cell inhibitory receptor or any combinations thereof;
(b) said NK cell activating receptor is at least one of Natural cytotoxicity triggering receptor 1 (NCR1, NKp46), Natural cytotoxicity triggering receptor 2 (NCR2, NKp44), Natural cytotoxicity triggering receptor 3 (NCR3, NKp30), tumor necrosis factor receptor superfamily 7 (TNFRSF7, CD27), Lymphocyte function-associated antigen 1 (LFA-1), cluster of differentiation 16 (CD16), NKG2D, Cytotoxic And Regulatory T Cell Molecule (CRTAM), DNAX Accessory Molecule-1 (DNAM-1), 2B4 (CD244, Cluster of Differentiation 244), killer cell lectin-like subfamily F, member 1 (KLRF1, NKp80) and any derivatives, splice variants, homologs and orthologs thereof, and wherein said NK cell inhibitory receptor is at least one of Killer-cell immunoglobulin-like receptor (KIR), Programmed cell death protein 1 (PD-1), Ly49, NKRP1A, CD94, NKG2A, T cell immunoreceptor with Ig and ITIM domains (TIGIT), CD96, T-cell immunoglobulin and mucin-domain containing-3 (TIM-3), Lymphocyte-activation gene 3 (LAG-3), Carcinoembryonic antigen-related cell adhesion molecule 1 (CEACAM1), cytotoxic T-lymphocyte-associated protein 4 (CTLA-4), Leukocyte-associated immunoglobulin-like receptor 1 (LAIR-1), Leukocyte immunoglobulin-like receptor subfamily B member 1 (LILRB1), and any derivatives, splice, homologs and orthologs variants thereof or any combinations thereof; and
(c) wherein each of said plurality of antibodies or aptamers specifically recognizes and binds an NK cell inhibitory or activating receptor of a plurality of inhibitory or activating receptors expressed by NK cells of a subject suffering from an immune-related disorder.

59. The at least one nano- or micro-particle, micellar formulation, vehicle or matrix according to claim 57, wherein said hematopoietic cell is a T cell, wherein said nano- or micro-particle, micellar formulation, vehicle or matrix is connected directly or indirectly by the outer nanoparticle surface thereof with at least one antibody, at least one aptamer or any combinations thereof, that specifically recognize and bind at least one of at least one T cell activating receptor and at least one T cell inhibitory receptor or any combinations thereof and wherein T cell activating receptor is any one of cluster of differentiation 3 (CD3), Cluster of Differentiation 28 (CD28), Cluster of Differentiation 69 (CD69), cluster of differentiation 4 (CD4), cluster of differentiation 8 (CD8), cluster of differentiation 137 (CD137), and T cell inhibitory receptor is at least one of Programmed cell death protein 1 (PD-1(, B- and T-lymphocyte attenuator (BTLA), luster of differentiation 160 (CD160), Cluster of Differentiation 244 (CD244, 2B4), cytotoxic T-lymphocyte associated protein 4 (CTLA-4), Lymphocyte-activation gene 3 (LAG-3), T cell immunoreceptor with Ig and ITIM domains (Tigit), Carcinoembryonic antigen-related cell adhesion molecule 1 (biliary glycoprotein) (CECAM), T cell immunoglobulin and mucin domain-containing protein 3 (TIM3), Leukocyte-associated immunoglobulin-like receptor 1 (Lair 1), Leukocyte immunoglobulin-like receptor subfamily B member 3 (LILRB3 or PirB), Platelet endothelial cell adhesion molecule (PECAM-1), cluster of differentiation-22 (CD22, Siglec 2), Sialic acid-binding Ig-like lectin 7 (Siglec 7), Sialic acid-binding Ig-like lectin 9 (Siglec 9), Killer cell lectin-like receptor subfamily G member 1 (KLRG1), Ig-Like Transcript 2 (ILT2), Killer-cell immunoglobulin-like receptor 2DL1 (KIR2DL/3DL), Cluster of Differentiation 72 (CD72), Cluster of Differentiation 94 (CD94, NKG2A) and cluster of differentiation 5 (CD5).

60. A pharmaceutical composition comprising as an active ingredient at least one combination according to claim 53, or any nano- or micro-particle, micellar formulation, vehicle, matrix or a cell comprising said combination, said composition optionally further comprises at least one of pharmaceutically acceptable carrier/s, excipient/s, auxiliaries, and/or diluent/s.

61. A method for activating at least one hematopoietic cell in an inhibitory immunological synapse (IS), the method comprising the step of contacting said hematopoietic cell with an activating effective amount of at least one combination comprising at least two compounds that specifically inhibit at least one of, the expression, activity and stability of at least one PTP and of at least one E3 ubiquitin-protein ligase, or any nano- or micro-particle, micellar formulation, vehicle or matrix, a composition or kit comprising said combination.

62. The method according to claim 61, wherein at last one of:
(a) said at least one PTP is at least one SHP protein, and wherein said at least one E3 ubiquitin-protein ligase is at least one member of the Cbl E3 ubiquitin-protein ligase family;
(b) said combination comprises at least one siRNA molecule specific for SHP-1, at least one siRNA molecule specific for Cbl-b and at least one siRNA molecule specific for c-Cbl, optionally, said siRNA molecule specific for SHP-1 comprise the nucleic acid sequence as denoted by at least one of SEQ ID NO: 9 and SEQ ID NO: 11, said siRNA molecule specific for Cbl-b comprise the nucleic acid sequence as denoted by at least one of SEQ ID NO: 1 and SEQ ID NO: 3, and said siRNA molecule specific for c-Cbl comprise the nucleic acid sequence as denoted by at least one of SEQ ID NO: 5 and SEQ ID NO: 7, or of any variants, homologs or derivatives thereof.

63. The method according to claim 61, wherein at least one of:
(a) said combination is comprised within at least one of a nano- or micro-particle, micellar formulation, vehicle or matrix or a composition;
(b) at least one targeting moiety is associated with the outer nanoparticle surface of said nano- or micro-particle, micellar formulation, vehicle or matrix; and
(c) said at least one targeting moiety is at least one of an antibody, an aptamer, a ligand or any combinations thereof, that specifically recognizes and binds at least one molecule expressed on the surface of at least one hematopoietic cell; and. (d) said nano- or micro-particle, micellar formulation, vehicle or matrix is associated directly or indirectly by the outer nanoparticle surface thereof with at least one of: at least one antibody, at least one aptamer or any combinations thereof, that specifically recognize and bind at least one of: (i) at least one hematopoietic cell activating receptor; and (ii) at least one hematopoietic cell inhibitory receptor; or any combinations thereof.

64. The method according to claim 61, wherein:
(a) said hematopoietic cell is an NK cell and wherein said NK cell activating receptor is at least one of NKp46, NKp44, NKp30, NKp80, CD27, LFA-1, CD16, NKG2D, CRTAM, DNAM-1, 2B4 (CD 244) and any derivatives and splice variants thereof, and wherein said NK cell inhibitory receptor is at least one of KIR, PD-1, Ly49, NKRP1A, CD94, NKG2A, TIGIT, CD96, TIM-3, LAG-3, CEACAM1, CTLA-4, LAIR-1, LILRB1, and any derivatives, splice variants, homologs and orthologs thereof or any combinations thereof; or
(b) said hematopoietic cell is a T cell, wherein said nano- or micro-particle, micellar formulation, vehicle or matrix is connected directly or indirectly by the outer nanoparticle surface thereof with at least one antibody, at least one aptamer or any combinations thereof, that specifically recognize and bind at least one of at least one T cell activating receptor and at least one T cell inhibitory receptor or any combinations thereof and wherein T cell activating receptor is any one of CD3, CD28, CD69, CD4, CD8, CD137, and T cell inhibitory receptor is at least one of PD-1, BTLA, CD160, 2B4, CTLA-4, LAG-3, Tigit, CECAM, Tim3, Lairl, PirB, PECAM1, CD22 (Siglec 2), Siglec 7, Siglec 9, KLRG1, ILT2, KIR2DL/3DL, CD72, CD94, NKG2A and CD5.

65. The method according to claim 61, wherein said cell is in a subject suffering from an immune-related disorder, and wherein said immune-related disorder is at least one of a cancer, a proliferative disorder, an infectious disease, a graft versus host disease, an inflammatory disorder, an immune-cell mediated disorder and an autoimmune disorder.

66. A method for treating, preventing, inhibiting, reducing, eliminating, protecting or delaying the onset of an immune-related disorder in a subject in need thereof, said method comprises administering to said subject a therapeutically effective amount of at least one combination comprising at least two compounds that specifically inhibit at least one of, the expression, activity and stability of at least one PTP protein and of at least one E3 ubiquitin-protein ligase, or any nano- or micro-particle, micellar formulation, vehicle, matrix, cell, a composition or kit comprising said combination, nano- or micro-particle, micellar formulation, vehicle, matrix or cell.

67. The method according to claim 66, wherein at least one of:
(a) said at least one PTP is at least one SHP protein, and wherein said at least one E3 ubiquitin-protein ligase is at least one member of the Cbl E3 ubiquitin-protein ligase family;
(b) said combination comprises at least one siRNA molecule specific for SHP-1, at least one siRNA molecule specific for Cbl-b and at least one siRNA molecule specific for c-Cbl; optionally, said siRNA molecule specific for SHP-1 comprise the nucleic acid sequence as denoted by at least one of SEQ ID NO: 9 and SEQ ID NO: 11, said siRNA molecule specific for Cbl-b comprise the nucleic acid sequence as denoted by at least one of SEQ ID NO: 1 and SEQ ID NO: 3, and said siRNA molecule specific for c-Cbl comprise the nucleic acid sequence as denoted by at least one of SEQ ID NO: 5 and SEQ ID NO: 7, or of any variants, homologs or derivatives thereof.

68. The method according to claim 66, wherein at least one of:
(a) said combination is comprised within at least one of a nano- or micro-particle, micellar formulation, vehicle or matrix, a cell or a composition;
(b) at least one targeting moiety is associated with the outer nanoparticle surface of said nano- or micro-particle, micellar formulation, vehicle or matrix; and
(c) said at least one targeting moiety associated with said nano- or micro-particle, micellar formulation, vehicle or matrix, is at least one of an antibody, an aptamer, a ligand or any combinations thereof, that specifically recognizes and binds at least one molecule expressed on the surface of at least one hematopoietic cell of said subject.

69. The method according to claim 66, wherein said hematopoietic cell is an NK cell of said subject, and wherein at least one of:
(a) said nano- or micro-particle, micellar formulation, vehicle or matrix is connected directly or indirectly by the outer nanoparticle surface thereof with at least one of at least one antibody, at least one aptamer or any combinations thereof, that specifically recognize and bind at least one of: (i) at least one NK cell activating receptor; and (ii) at least one NK cell inhibitory receptor or any combinations thereof, wherein said at least one of activating or inhibitory receptors and any combinations thereof are expressed by NK cells of said subject;
(b) said NK cell activating receptor is at least one of NKp46, NKp44, NKp30, NKp80, CD27, LFA-1, CD16, NKG2D, CRTAM, DNAM-1, 2B4 (CD244) and any derivatives and splice variants thereof, and wherein said NK cell inhibitory receptor is at least one of KIR, PD-1, Ly49, NKRP1A, CD94, NKG2A, TIGIT, CD96, TIM-3, LAG-3, CEACAM1, CTLA-4, LAIR-1, LILRB1, and any derivatives, splice variants, homologs and orthologs thereof or any combinations thereof; and
(c) said nanoparticle is connected directly or indirectly with a plurality of antibodies, aptamers or any combinations thereof, wherein each of said plurality of antibodies or aptamers specifically recognizes and binds an NK cell inhibitory or activating receptor of a plurality of inhibitory or activating receptors expressed by NK cells of said subject.

70. The method according to claim 66, wherein said hematopoietic cell is a T cell, wherein said nano- or micro-particle, micellar formulation, vehicle or matrix is connected directly or indirectly by the outer nanoparticle surface thereof with at least one antibody, at least one aptamer or any combinations thereof, that specifically recognize and bind at least one of at least one T cell activating receptor and at least one T cell inhibitory receptor or any combinations thereof and wherein T cell activating receptor is any one of CD3, CD28, CD69, CD4, CD8, CD137, and T cell inhibitory receptor is at least one of PD-1, BTLA, CD160, 2B4, CTLA-4, LAG-3, Tigit, CECAM, Tim3, Lairl, PirB, PECAM1, CD22 (Siglec 2), Siglec 7, Siglec 9, KLRG1, ILT2, KIR2DL/3DL, CD72, CD94, NKG2A and CD5.

71. The method according to claim 66, wherein said subject is suffering from an immune-related disorder, said immune-related disorder is at least one of a viral infection, cancer, a proliferative disorder, a graft versus host disease, an inflammatory disorder, an immune-cell mediated disorder and an autoimmune disorder.

72. A kit comprising at least one combination according to claim 53, wherein said kit comprises:
(a) at least one compound that specifically inhibit at least one of, the expression, activity and stability of at least one SHP protein, or any nano- or micro-particle, micellar formulation, vehicle, matrix, cell or composition comprising said compound;
(b) at least one compounds that specifically inhibit at least one of, the expression, activity and stability of at least one member of the Cbl family, or any nano- or micro-particle, micellar formulation, vehicle, matrix, cell or composition comprising said compound; and optionally
(c) at least one additional therapeutic agent.
